SEQUENCE LISTING (8 sequences)

SEQ ID NO: 1 - protein, 227 aa, synthetic construct
  REGION 1..227: note = Description of sequence: CMP-sialic acid synthetase
EKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILHHKES

SEQ ID NO: 2 - protein, 266 aa, synthetic construct
  REGION 1..266: note = Description of sequence: sialyltransferase
DKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLVDKLAAALE

SEQ ID NO: 3 - protein, 257 aa, synthetic construct
  REGION 1..257: note = Description of sequence: sialyltransferase
KKVIIAGNGPSLKEIDYSRLPNDFDVFRCNQFYFEDKYYLGKKCKAVFYNPSLFFEQYYTLKHLIQNEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLMSNFIIQEKNNYTKDILIPSSEAYGKFSKNIN

SEQ ID NO: 4 - DNA, 2049 bp, synthetic construct
  misc_feature 1..2049: note = Description of sequence: MBP-CjCst-I-Delta-145-His6 coding sequence
  source 1..2049: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatgacacgcaccagaatggagaacgagttgattgtctcaaagaacatgcaaaacatcattatcgcaggcaatggccccagcctcaagaatattaattacaagcgtctcccgcgggaatacgacgttttccgttgtaaccaattttacttcgaggacaagtattacttagggaagaagataaaggcagttttctttaaccctggcgtattcttacaacaataccacaccgctaagcaacttatactcaagaatgaatacgagatcaagaacattttctgttccactttcaatctcccgtttattgagagcaacgatttcctgcaccaattttacaacttcttccccgatgcaaaactgggctacgaggttatcgagaacctgaaggaattttacgcctacatcaagtataatgagatatactttaacaagcgtatcacatccggggtatatatgtgtgccatcgccatagccctgggttacaagaccatttacttgtgcggaattgacttctacgagggcgatgtgatctatccattcgaggctatgtcaactaatattaagaccatcttcccgggcattaaagactttaagccgagcaattgtcatagtaaagagtacgacatcgaagcattaaaacttctgaaatcgatatacaaggtgaatatatacgcgctctgtgacgactccatattagcaaaccatttccctttgagcatcaacatcaataacaatttcacattggagaacaagcacaacaatagtatcaacgacatcctgttaactgacaacacaccgggtgtttcattttacaagaatcaactgaaagcagataacaagataatgctgaacttttatcatcatcatcatcatcattaa

SEQ ID NO: 5 - protein, 682 aa, synthetic construct
  REGION 1..682: note = Description of sequence: MBP-CjCst-I-Delta-145-His6
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMTRTRMENELIVSKNMQNIIIAGNGPSLKNINYKRLPREYDVFRCNQFYFEDKYYLGKKIKAVFFNPGVFLQQYHTAKQLILKNEYEIKNIFCSTFNLPFIESNDFLHQFYNFFPDAKLGYEVIENLKEFYAYIKYNEIYFNKRITSGVYMCAIAIALGYKTIYLCGIDFYEGDVIYPFEAMSTNIKTIFPGIKDFKPSNCHSKEYDIEALKLLKSIYKVNIYALCDDSILANHFPLSININNNFTLENKHNNSINDILLTDNTPGVSFYKNQLKADNKIMLNFYHHHHHH

SEQ ID NO: 6 - protein, 6 aa, synthetic construct
  REGION 1..6: note = Description of sequence: histidine tag (His6 tag)
HHHHHH

SEQ ID NO: 7 - protein, 8 aa, synthetic construct
  REGION 1..8: note = Description of sequence: Strep tag
WSHPQFEK

SEQ ID NO: 8 - protein, 8 aa, synthetic construct
  REGION 1..8: note = Description of sequence: FLAG peptide
DYKDDDDK

(19) United States
(12) Patent Application Publication
(10) Pub. No.: US 2024/0409571 A1
(43) Pub. Date: Dec. 12, 2024

(54) CHEMOENZYMATIC SYNTHESIS OF N-ACETYLATED GANGLIOSIDES AND GLYCOSPHINGOSINES

(71) Applicant: The Regents of the University of California, Oakland, CA (US)

(72) Inventors: Xi CHEN, Davis, CA (US); Hai YU, Davis, CA (US); Bijoyananda MISHRA, Davis, CA (US); Zimin ZHENG, Davis, CA (US); Anand Kumar AGRAHARI, Davis, CA (US); Arin GUCCHAIT, Davis, CA (US)

(73) Assignee: The Regents of the University of California, Oakland, CA (US)

(21) Appl. No.: 18/627,308

(22) Filed: Apr. 4, 2024

Related U.S. Application Data
(60) Provisional application No. 63/471,907, filed on Jun. 8, 2023.

(51) Int. Cl. C07H 15/12; C07H 1/00; C12P 13/00

(52) U.S. Cl. CPC C07H 15/12; C07H 1/00; C12P 13/001

(57) ABSTRACT

Described herein are novel stable 9-N-, 8-N-, and 7-N-acetyl analogues of instable 9-O-acetyl, 8-O-acetyl, and 7-O-acetyl b-series gangliosides and glycosphingosines, including GD3, GD2, GD1b, GT1b, GQ1b, and their glycosphingosines. Chemoenzymatic methods for the production of the stable 9-N-, 8-N-, and 7-N-acetyl analogues are also described herein.

### CROSS-REFERENCE TO RELATED APPLICATION

The present application claims the benefit of and the priority to U.S. Provisional Application No. 63/471,907, filed on Jun. 8, 2023, which is hereby incorporated by reference in its entirety for all purposes.

### STATEMENT REGARDING FEDERALLY FUNDED RESEARCH

This invention was made with government support under Grant Nos. R01AI130684 and R44GM139441, awarded by the National Institutes of Health, and under Grant No. U01GM120419 awarded by the National Institutes of Health Common Fund Glycoscience Program Award. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Apr. 3, 2024, is entitled “081906-1440657_253110US_ST26.xml”, and is 14,305 bytes in size.

### BACKGROUND

Gangliosides are biologically important sialic acid (Sia)-containing glycosphingolipids. Those in the ganglio-series containing a disialylated lactosyl ceramide core with an α2-8-linked terminal sialic acid are called b-series gangliosides which include GD3, GD2, GD1b, GT1b, and more complex GQ1b and GQ1bα. GD3 (also called CD60a) and GD2 are oncofetal markers and are among the four gangliosides (two others are gangliosides GM3 and fucosyl GM1) on the list of prioritized cancer antigens while GD1b and GT1b are among the four most abundant gangliosides in vertebrate brains (two others are a-series gangliosides GM1a, which is also called GM1, and GD1a).

N-Acetylneuraminic acid (Neu5Ac) is the major sialic acid form in gangliosides. The terminal α2-8-linked Neu5Ac of the disialylated lactosyl ceramide core in the b-series gangliosides can be modified by O-acetylation, with 9-O-acetyl Neu5Ac (Neu5,9Ac₂) as the most common O-acetylated form. 90Ac-GD3, which is also called CD60b, was shown to be expressed selectively on the neuroepithelial cells of developing rat cerebellum, in perinatal rat retina, and on human malignant melanoma cells. It was shown to protect cells including glioblastoma cells, lymphoblasts, and lymphocytes from apoptosis including that is induced by GD3. On the contrary, it was shown to induce apoptosis in mature erythrocytes. 90Ac-GD3 has also been shown to be involved in modulating cell motility such as neuronal and tumor cell migration, as well as neurite outgrowth.

Different from GD2 which is expressed in peripheral nerve fibers in addition to its cancer cell expression, 9-O-acetylated GD2 (90Ac-GD2) is more selectively expressed by cancer cells such as neuroblastoma, glioblastoma, and breast cancer cells. Therefore, 90Ac-GD2 is predicted to be a more selective anti-cancer immunotherapeutic target than GD2. A recent mass spectrometry-based profiling approach showed that O-acetylation could occur on the inner in addition to the terminal Sia in gangliosides.

90Ac-GD1b (also called “neurostatin”) is a potent inhibitor of astroblast and astrocytoma proliferation and also induces necrosis at nanomolar concentrations. More recently, 90Ac-GD1b has been tested to treat neuronal inflammation and has shown strong anti-inflammatory effects even at nanomolar concentrations. 90Ac-GD1b can be potentially used as an acceptor substrate by ST3GAL II to form 90Ac-GT1b.

Despite the recognition of the important functions of O-acetylated gangliosides, their specific biological roles are not clear. This is mainly due to the instability of Sia 0-acetyl group towards pH change and/or esterase activities which presents special challenges for elucidating functions and developing applications for structurally complex O-acetylated b-series gangliosides.

### SUMMARY

Described herein are 9-O-acetylated b-series gangliosides and their N-acetyl analogues, along with methods of making the same. The stable N-acetyl analogues of biologically important 9-O-acetylated b-series gangliosides, including 9NAc-GD3, 9NAc-GD2, 9NAc-GD1b, and 9NAc-GT1b, were chemoenzymatically synthesized from a GM3 sphingosine. Two chemoenzymatic methods using either 6-azido-6-deoxy-N-acetylmannosamine (ManNAc6N₃) as a chemoenzymatic synthon or 6-acetamido-6-deoxy-N-acetylmannosamine (ManNAc6NAc) as an enzymatic precursor for 9-acetamido-9-deoxy-N-acetylneuraminic acid (Neu5Ac9NAc) were developed and compared for the synthesis of 9NAc-GD3. In some examples, the latter method was found to be more efficient and was used to produce the desired 9-N-acetylated glycosylsphingosines. Furthermore, glycosylsphingosine acylation reaction conditions were improved to obtain target 9-N-acetylated gangliosides in a faster reaction with an easier purification process compared to the previous acylation conditions.

### Described Herein is a Compound of Formula I:

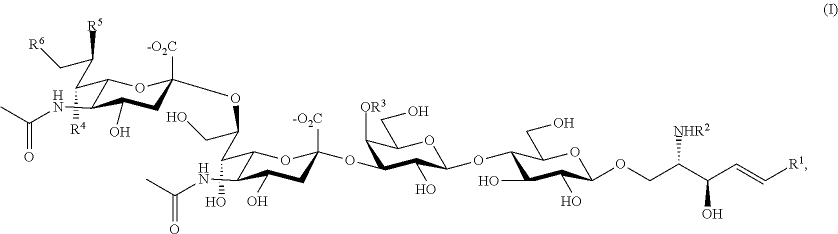

or a salt thereof, wherein R¹ is an alkyl chain; R² is H, —C(O)alkyl, alkyl, —C(O)alkenyl, alkenyl, —C(O)alkynyl, or alkynyl; R³ is H, a monosaccharide, or an oligosaccharide; and R⁴, R⁵, and R⁶ are each independently selected from OH, NHAc, NH₂, and N₃, wherein at least one of R⁴, R⁵, and R⁶ is NHAc, NH₂, or N₃. Optionally, R¹ is C₁-C₂₅ alkyl (e.g., —C₁₃H₂₇ or —C₁₅H₃₁). In some cases, R² is —C(O)C₁-C₂₅ alkyl, —C₁-C₂₅ alkyl, —C(O)C₂-C₂₅ alkenyl, —C₂-C₂₅ alkenyl, —C(O)C₂-C₂₅ alkynyl, or —C₂-C₂₅ alkynyl. Optionally, R² is —C(O)C₁₅H₃₁, —C(O)C₁₇H₃₅, or —C(O)C₁₉H₃₉. In some cases, R³ comprises a substituted or unsubstituted GalNAc. Optionally, R³ is GalNAc, Gal-GalNAc, Neu5Ac-Gal-GalNAc, or Neu5Ac-Neu5Ac-Gal-GalNAc. In some cases, R³ is H.

Also described herein are methods of synthesizing an N-acetyl-containing b-series ganglioside, comprising step (i) forming a reaction mixture comprising a donor precursor, cytidine 5′-triphosphate, pyruvate, a sialic acid aldolase, a CMP-sialic acid synthetase, a sialyltransferase, and an acceptor comprising an a-series sphingosine, under conditions sufficient to result in the sialylation of the a-series sphingosine to form a b-series sphingosine of the following formula:

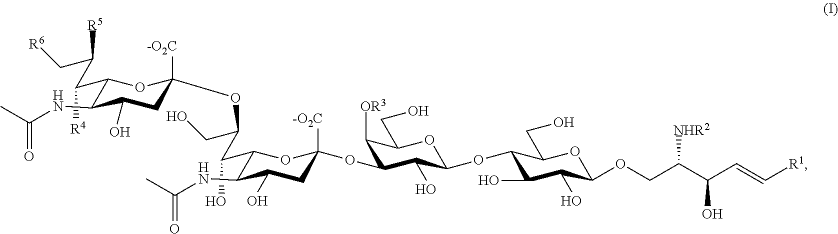

or a salt thereof, wherein R¹ is an alkyl chain; R² is H; R³ is H, a monosaccharide, or an oligosaccharide; and R⁴, R⁵, and R⁶ are each independently selected from OH, NHAc, or N₃, wherein at least one of R⁴, R⁵, and R⁶ is NHAc or N₃; and step (ii) acetylating the b-series sphingosine to form a b-series ganglioside of Formula I:

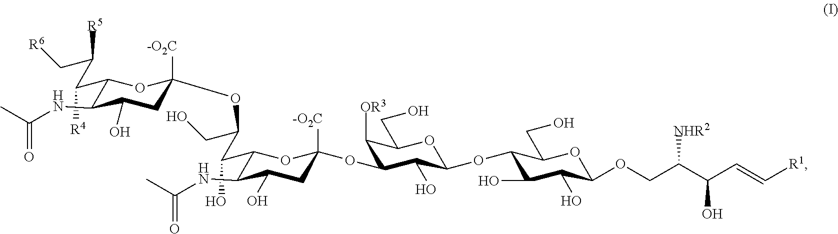

or a salt thereof, wherein R¹ is an alkyl chain; R² is —C(O)alkyl; R³ is H, a monosaccharide, or an oligosaccharide; R⁴, R⁵, and R⁶ are each independently selected from OH, NHAc, or N₃, wherein at least one of R⁴, R⁵, and R⁶ is NHAc or N₃. Optionally, the donor precursor comprises an N-acetyl group or an azido group. In some cases, the donor precursor is 6-acetamido-6-deoxy-N-acetylmannosamine (ManNAc6NAc), 6-azido-6-deoxy-N-acetylmannosamine (ManNAc6N₃), 4-acetamido-4-deoxy-N-acetylmannosamine (ManNAc4NAc), 4-azido-4-deoxy-N-acetylmannosamine (ManNAc4N₃), 5-acetamido-5-deoxy-N-acetylmannosamine (ManNAc5NAc), or 5-azido-5-deoxy-N-acetylmannosamine (ManNAc5N₃). Optionally, the acceptor comprises GM3βSph.

In some cases, the sialic acid aldolase is P. multocida sialic acid aldolase (PmAldolase) or Escherichia coli sialic acid aldolase (EcAldolase). In some cases, wherein the CMP sialic acid synthetase is Neisseria meningitidis CMP-sialic acid synthetase (NmCSS) or Legionella pneumophila CMP-5,7-di-N-acetyllegionaminic acid synthetase (LpCLS). Optionally, the sialyltransferase is Campylobacter jejuni α2-3/8-sialyltransferase (CjCst-II).

The method can further comprise one or more glycosylation steps following step (i). In some cases, when the donor precursor is 6-azido-6-deoxy-N-acetylmannosamine (ManNAc6N₃), 4-azido-4-deoxy-N-acetylmannosamine (ManNAc4N₃) or 5-azido-5-deoxy-N-acetylmannosamine (ManNAc5N₃), the method further comprises a step of reducing the azido group to an amino group after step (ii).

The method can further comprise one or more glycosylation steps following step (i). In some cases, when the donor precursor is 6-azido-6-deoxy-N-acetylmannosamine (ManNAc6N₃), 4-azido-4-deoxy-N-acetylmannosamine (ManNAc4N₃) or 5-azido-5-deoxy-N-acetylmannosamine (ManNAc5N₃), the method further comprises a step of converting the azido group to an acetamido group after step (ii).

The details of one or more embodiments are set forth in the drawings and the description below. Other features, objects, and advantages will be apparent from the description and drawings, and from the claims.

### DESCRIPTION OF THE DRAWINGS

FIG. 1 is a schematic containing structures of 0-, a-, b-, and c-series gangliosides with the locations of O-acetyl and/or N-acetyl sialic acids indicated by asterisks.

FIG. 2 contains structures of A. N-Acetylneuraminic acid (Neu5Ac) as well as 9-O-acetyl Neu5Ac (Neu5,9Ac₂) and its 9-N-acetyl analog, 9-acetamido-9-deoxy-N-acetylneuraminic acid (Neu5Ac9NAc); and B. 9-N-acetylated b-series gangliosides including 9NAc-GD3 (1), 9NAc-GD2 (2), 9NAc-GD1b (3), and 9NAc-GT1b (4) synthesized according to the present application.

FIGS. 3A-3D contain high resolution mass spectrometry (HRMS) results for the synthesis of (3A) 9NAc-GD3βSph (7), (3B) 9NAc-GD2βSph (8), (3C) 9NAc-GD1bβSph (9), and (3D) 9NAc-GT1bβSph (10).

FIG. 4 contains TLC results for comparing reaction conditions for the formation of GM3 ganglioside from GM3 sphingosine by acylation. Lanes: 1, GM3βSph standard; and reaction mixture of 2, 50 mM carbonate buffer (pH 9.2)/THF; 3, 100 mM CAPS buffer (pH 9.5)/THF; 4, 1% Na₂CO₃/THF; and 5, sat. NaHCO₃/THF. TLC developing solvent used was i-PrOH:H₂O:NH₄OH=10:2:1 (by volume).

FIGS. 5A-5I contain ¹H-NMR and ¹³C-NMR spectra for compounds 5A. 9N₃-GD3βSph (6); 5B. 9NAc-GD3βSph (7); 5C. 9NAc-GD2βSph (8); 5D. 9NAc-GD1bβSph (9); 5E. 9NAc-GT1bβSph (10); 5F. 9NAc-GD3 (1); 5G. 9NAc-GD2 (2); 5H. 9NAc-GD1b (3); and 51. 9NAc-GT1b (4).

FIG. 6 contains the ¹H-NMR and ¹³C-NMR spectra for compound (15).

FIG. 7 contains the ¹H-NMR and ¹³C-NMR spectra for compound ManNAc4N₃ (16).

FIGS. 8A-8BB contains ¹H-NMR, ¹³C-NMR, and HRMS spectra for compounds synthesized in Example 4 (“Chemical Synthesis of ManNAc5N₃, ManNAc5,6diN₃, and Neu5Ac8NAc”).

### DETAILED DESCRIPTION

O-Acetylation is a common modification of sialic acids (Sias), which are frequently found as terminal monosaccharides of glycoconjugates in humans, other vertebrates, and some pathogenic bacteria that infect vertebrates. Such O-acetyl groups on sialic acids play multiple roles in immunology, oncology, virology, bacterial and viral infection, and neuroscience. However, they are labile and prone to migration under physiological conditions, making them challenging to study. The 9-O-acetyl group on sialic acids can migrate to C8-OH and C7-OH position at pH above 5, and synthetic N-acetyl analogs are stable mimics of O-acetyl sialosides.

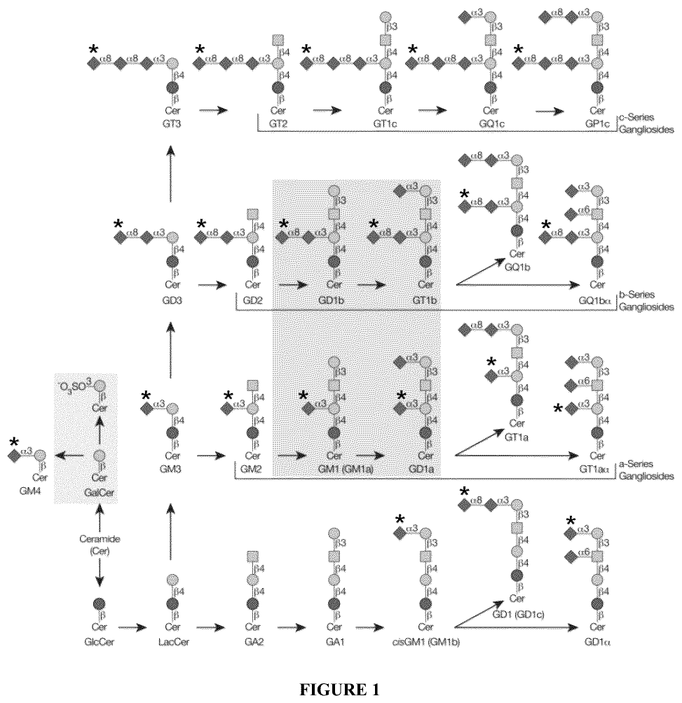

The functions of b-series gangliosides (sialic acid-containing glycosphingolipids) carrying 9-O-acetyl group at the terminal α2-8-linked Sia have significant therapeutic impact. These gangliosides, including 9-O-acetyl GD3, GD2, and GD1b, have shown promise in developing cancer immunotherapy; regulating cell apoptosis; influencing cell motility, migration, and neurite outgrowth; inhibiting astroblast and astrocytoma cell proliferation; and anti-inflammatory reagents. However, the detailed mechanisms are not fully elucidated and their applications are not fully explored due to the instability of O-acetyl groups and the lack of tools for their investigation. Described herein are chemoenzymatic methods for the production of the stable 9-N-, 8-N-, and 7-N-acetyl analogues of instable 9-O-acetyl GD3, GD2, GD1b, GT1b, and GQ1b. N-acetylation of terminal α2-8-linked Sia on these gangliosides will not alter the three-dimensional structure, but will enhance the strength and/or duration of functional impacts of the corresponding O-acetyl gangliosides. Chemoenzymatic methods for the synthesis of 9-O-acetyl GD3, GD2, GD1b, GT1b, and GQ1b are also described. Similar methods can be applied for the synthesis of other gangliosides including those in the 0-series, a-series, and c-series gangliosides (FIG. 1). The corresponding O-acetyl glycosyl sphingosines and their N-acetyl analogues are also described herein.

### Definitions

As used herein, the term “monosaccharide” refers to a sugar having a six-membered carbon backbone (i.e., a hexose). Examples of monosaccharides include, but are not limited to, glucose (Glc), galactose (Gal), mannose (Man), glucuronic acid (GlcA), and iduronic acid (IdoA). Monosaccharides also include hexoses substituted with hydroxy groups, oxo groups, amino groups, acetamido groups, and other functional groups. “Deoxy” monosaccharides refer to monosaccharides having carbon atoms one or more carbon atoms in the hexose backbone having only hydrogen substituents. Monosaccharides also include, but are not limited to, glucosamine (2-amino-2-deoxy-glucose; GlcN), N-acetylglucosamine (2-acetamido-2-deoxy-glucose; GlcNAc), galactosamine (2-amino-2-deoxy-galactose; GalN;), N-acetylgalactosamine (2-acetamido-2-deoxy-galactose; GalNAc), mannosamine (2-amino-2-deoxy-mannose; ManN), N-acetylmannosamine (2-acetamido-2-deoxy-mannose; ManNAc), and sialic acids such as N-acetylneuraminic acid (Neu5Ac) and its derivatives.

As used herein, the term “oligosaccharide” refers to a compound containing at least two sugars covalently linked together. Oligosaccharides include disaccharides, trisaccharides, tetrasaccharides, pentasaccharides, hexasaccharides, heptasaccharides, octasaccharides, and the like. Covalent linkages for linking sugars generally consist of glycosidic linkages (i.e., C—O—C bonds) formed from the hydroxyl groups of adjacent sugars. Linkages can occur between the 2-carbon and the 3-carbon of adjacent sugars (i.e., α2-3 sialyl linkage), the 2-carbon and the 8-carbon of adjacent sugars (i.e., α2-8-sialyl linkage), or the 2-carbon and the 6 carbon of adjacent sugars (i.e., α2-6-sialyl linkage).

As used herein, the term “isomer” refers to a compound having the same bond structure as a reference compound but having a different three-dimensional arrangement of the bonds. An isomer can be, for example, an enantiomer or a diastereomer.

As used herein, the term “glycoside” refers to a saccharide compound having a moiety “—OR” replacing a hydroxyl group of the parent compound, wherein R is another saccharide (e.g., a monosaccharide, oligosaccharide, or polysaccharide) or a non-saccharide moiety (e.g., a lipid, a protein, a peptide, a linker moiety, a label moiety, etc.). In some embodiments, the moiety —OR in the glycoside replaces the hydroxyl group of the anomeric carbon at the reducing end of the parent saccharide.

A “galactoside” refers to a galactopyranose moiety or a galactofuranose moiety wherein the anomeric hydroxyl group of the parent compound is replaced with a moiety —OR as described above. Galactosides include, for example, lactosides (i.e., P-D-galactopyranosyl-(1→4)-D-glucopyranoses).

An “N-acetylgalactosaminide” refers to a galactopyranose moiety or a galactofuranose moiety wherein the anomeric hydroxyl group of the parent compound is replaced with a moiety —OR as described above, and wherein at least one additional hydroxyl group of the parent compound is replaced with —NC(O)R′, wherein R′ is C₁₋₆ alkyl or C₁₋₆ hydroxyalkyl. N-Acetylgalactosaminides include, for example, N-acetylgalactosamine (GalNAc)-derived 2-acetamido-2-deoxy-D-galactopyranosides.

A “glucoside” refers to a glucopyranose moiety or a glucofuranose moiety wherein the anomeric hydroxyl group of the parent compound is replaced with a moiety —OR as described above.

An “N-acetylglucosaminide” refers to a glucopyranose moiety or a glucofuranose moiety wherein the anomeric hydroxyl group of the parent compound is replaced with a moiety —OR as described above, and wherein at least one additional hydroxyl group of the parent compound is replaced with —NC(O)R′, wherein R′ is C₁₋₆ alkyl or C₁₋₆ hydroxyalkyl. N-Acetylglucosaminides include, for example, N-acetylglucosamine (GlcNAc)-derived 2-acetamido-2-deoxy-D-glucopyranosides.

A “sialoside” refers to a sialic acid moiety wherein the anomeric hydroxyl group of the parent compound is replaced with a moiety —OR as described above. Sialic acid is a general term for N- and 0-substituted derivatives of neuraminic acid or 2-keto-3-deoxy-D-glycero-D-galacto-nononic acid (Kdn), and includes, but is not limited to, N-acetyl (Neu5Ac) or N-glycolyl (Neu5Gc) substitutions, as well as 0-substitutions including acetyl, lactyl, methyl, sulfate and phosphate, among others.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 30 carbons atoms, such as, but not limited to heptyl, octyl, nonyl, decyl, etc. The term “alkylene” refers to a divalent alkyl radical, wherein the two points of attachment to the diradical are on the same carbon atom or different carbon atoms.

As used herein, the terms “halo” and “halogen” refer to a fluorine, chlorine, bromine, or iodine atom.

As used herein, the term “sialic acid aldolase” refers to an aldolase that catalyzes a reversible reaction that converts a suitable hexosamine, hexose, pentose, or derivative (such as N-acetyl mannosamine) to sialic acid via reaction with pyruvate.

As used herein, the term “CMP-sialic acid synthetase” refers to a polypeptide that catalyzes the synthesis of cytidine monophosphate sialic acid (CMP-sialic acid) from cytidine triphosphate (CTP) and sialic acid.

As used herein, the term “sialyltransferase” refers to an enzyme that catalyzes the transfer of a sialic acid to a monosaccharide, an oligosaccharide, or another glycosylated molecule.

As used herein, the term “sialidase” refers to an enzyme that catalyzes the cleavage of a terminal sialic acid from a sialylated target such as an oligosaccharide, a polysaccharide, or a glycosylated protein.

The term “variant,” in the context of the enzymes in the present disclosure, means a polypeptide, typically recombinant, that comprises one or more amino acid substitutions relative to a corresponding, naturally-occurring or unmodified sialyltransferase.

The term “amino acid” refers to any monomeric unit that can be incorporated into a peptide, polypeptide, or protein. Amino acids include naturally-occurring a-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, y-carboxyglutamate and O-phosphoserine. Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

The terms “amino acid modification” and “amino acid alteration” refer to a substitution, a deletion, or an insertion of one or more amino acids. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). “Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

In certain embodiments, an enzyme variant will have at least about 80%, e.g., at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, sequence identity to any one of the amino acid sequences set forth herein.

In some embodiments, the polypeptide further comprises one or more heterologous amino acid sequences located at the N-terminus and/or the C-terminus of the polypeptide. The polypeptide can contain a number of heterologous sequences that are useful for expressing, purifying, and/or using the polypeptide. The polypeptide can contain, for example, a poly-histidine tag (e.g., a His6 tag (SEQ ID NO: 6)); a calmodulin-binding peptide (CBP) tag; a NorpA peptide tag; a Strep tag (e.g., Trp-Ser-His-Pro-Gln-Phe-Glu-Lys (SEQ ID NO: 7)) for recognition by/binding to streptavidin or a variant thereof, a FLAG peptide (i.e., Asp-Tyr-Lys-Asp-Asp-Asp-Asp-Lys (SEQ ID NO: 8)) for recognition by/binding to anti-FLAG antibodies (e.g., M1, M2, M5); a glutathione S-transferase (GST); or a maltose binding protein (MBP) polypeptide.

As used herein, the term “forming a reaction mixture” refers to the process of bringing into contact at least two distinct species such that they mix together and can react, either modifying one of the initial reactants or forming a third distinct species, i.e., a product. It should be appreciated, however, the resulting reaction product can be produced directly from a reaction between the added reagents or from an intermediate from one or more of the added reagents which can be produced in the reaction mixture.

### N-Acetylated Gangliosides and Glycosphingosines

The N-acetylated sialic acid containing gangliosides and glycosphingosines described herein can be used for the study of biological and pathological processes immunology, oncology, virology, neuroscience, and other fields. The N-acetylated sialic acid-containing gangliosides and glycosphingosines, in particular, can be used as mimics of O-acetyl sialic acids without the instability associated with O-acetylation.

Described herein are compounds according to Formula I:

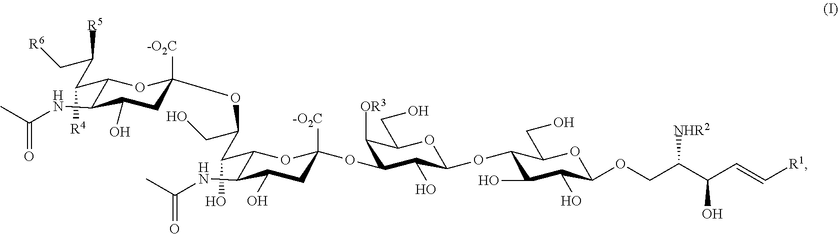

or a salt thereof.

In Formula I, R¹ is an alkyl chain. Optionally, R¹ is C₁-C₂₅ alkyl, such as —C₁₃H₂₇ or —C₁₅H₃₁.

Also in Formula I, R² is H, —C(O)alkyl, alkyl, —C(O)alkenyl, alkenyl, —C(O)alkynyl, or alkynyl. Optionally, R² is —C(O)C₁-C₂₅ alkyl, —C₁-C₂₅ alkyl, —C(O)C₂-C₂₅ alkenyl, —C₂-C₂₅ alkenyl, —C(O)C₂-C₂₅ alkynyl, or —C₂-C₂₅ alkynyl. In some cases, R² is —C(O)C₁₅H₃₁, —C(O)C₁₇H₃₅, or —C(O)C₁₉H₃₉.

Additionally in Formula I, R³ is H, a monosaccharide, or an oligosaccharide. Optionally, R³ includes a substituted or unsubstituted GalNAc. In some examples, R³ can be GalNAc. In other examples, R³ can be Gal-GalNAc. In still other examples, R³ can be Neu5Ac-Gal-GalNAc. In still further examples, R³ can be Neu5Ac-Neu5Ac-Gal-GalNAc. Optionally, R3 is H.

Further in Formula I, R⁴, R⁵, and R⁶ are each independently selected from OH, NHAc, NH₂, and N₃, wherein at least one of R⁴, R⁵, and R⁶ is NHAc, NH₂, or N₃.

### N-Acetyl Gangliosides

In some examples, the compounds described herein are N-acetyl gangliosides. Optionally, the compound is a GD3-analog selected from the group consisting of

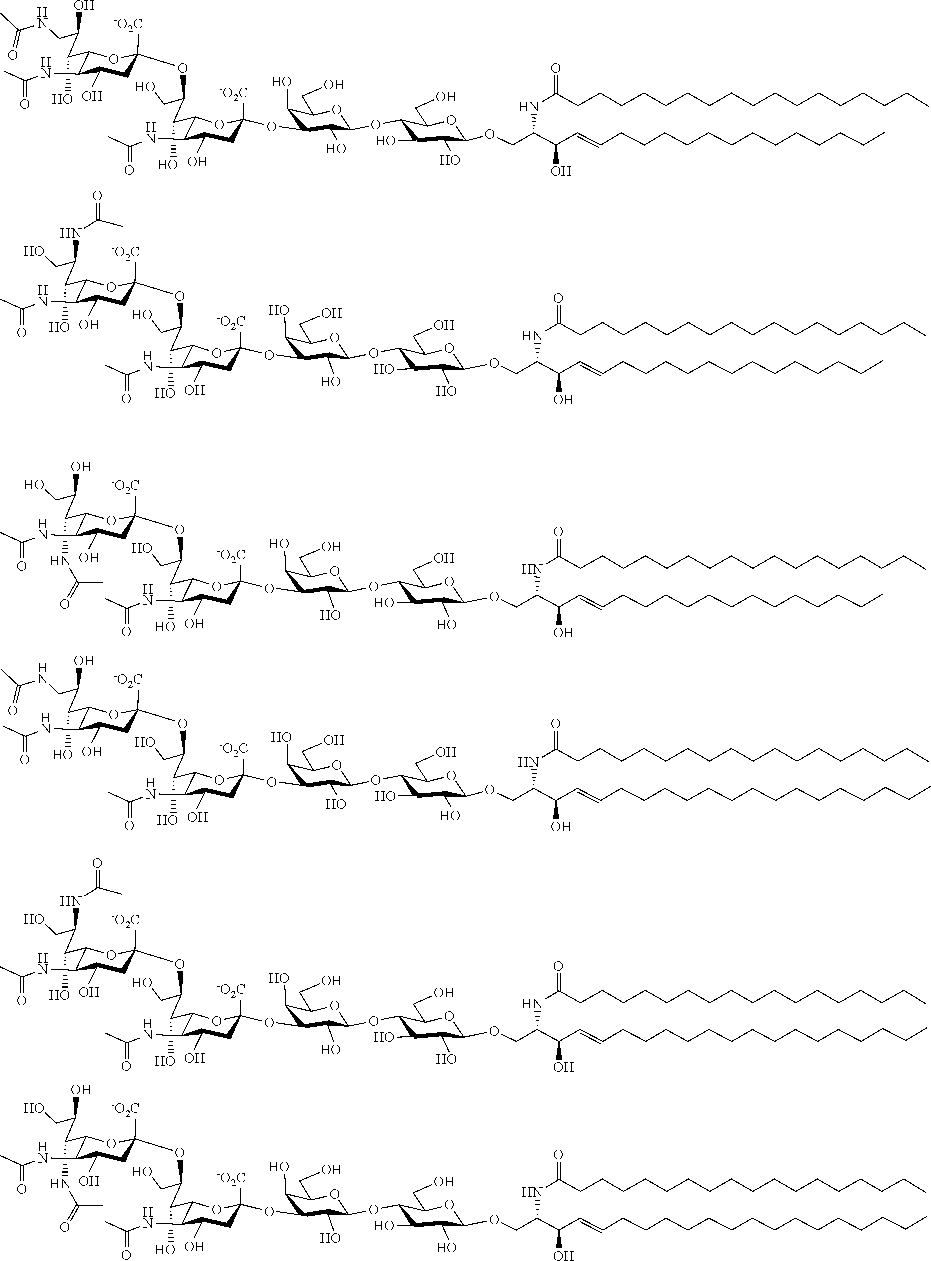

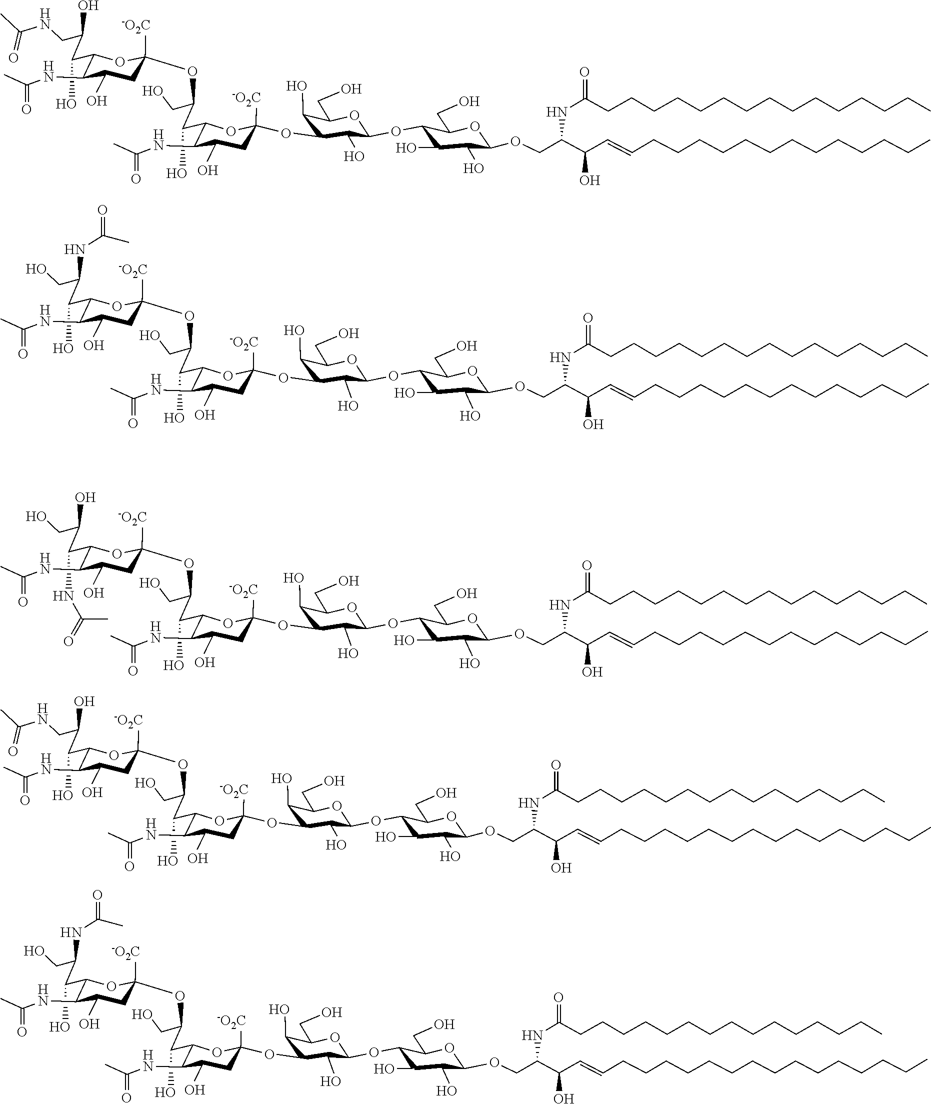

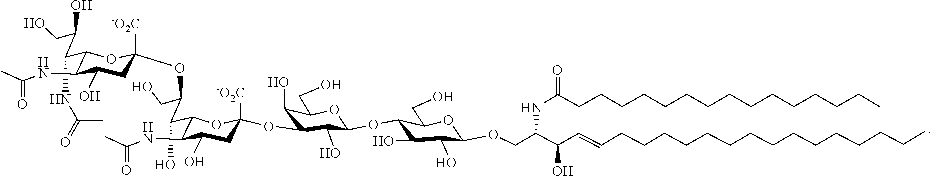

Optionally, the compound is a GD2-analog selected from the group consisting of

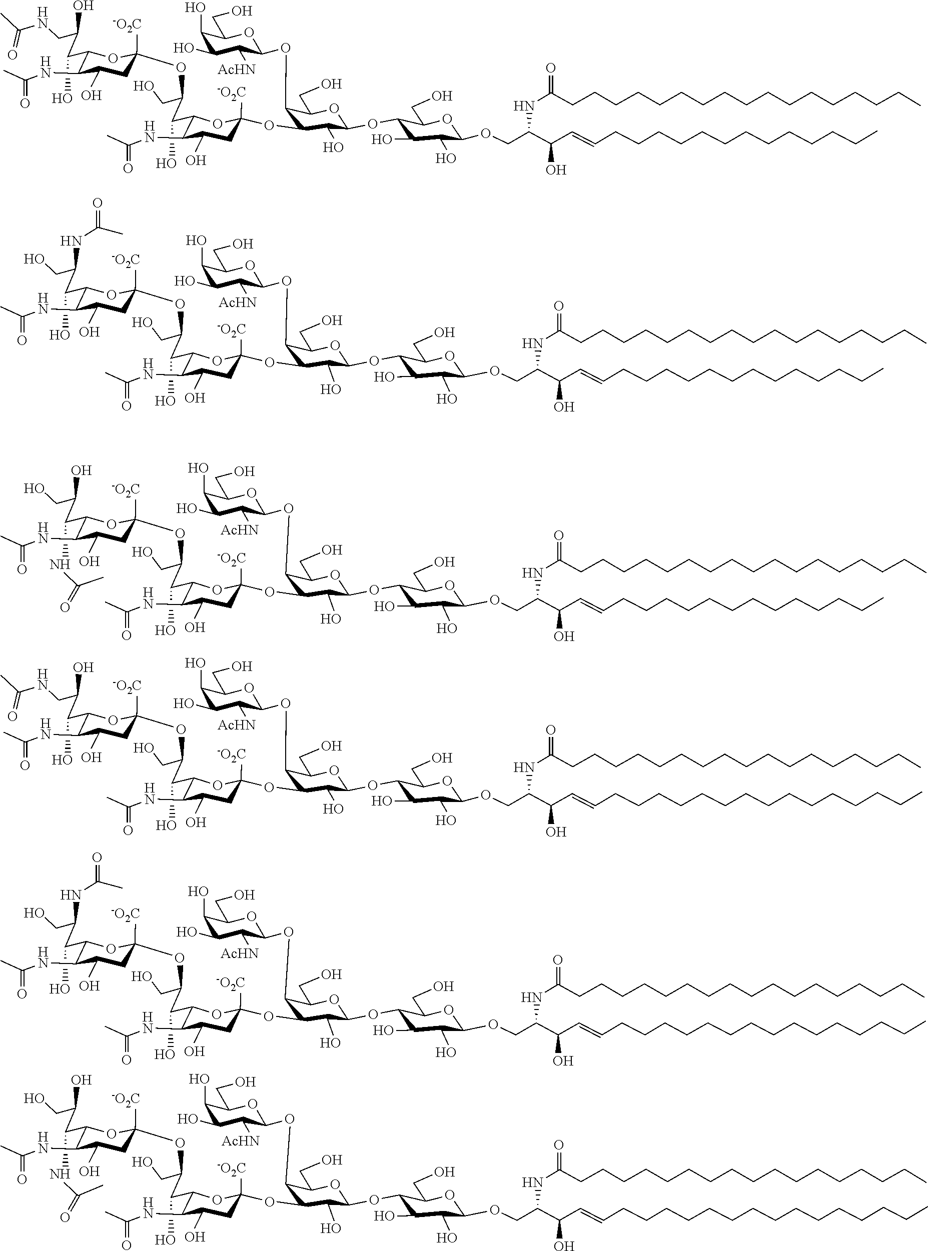

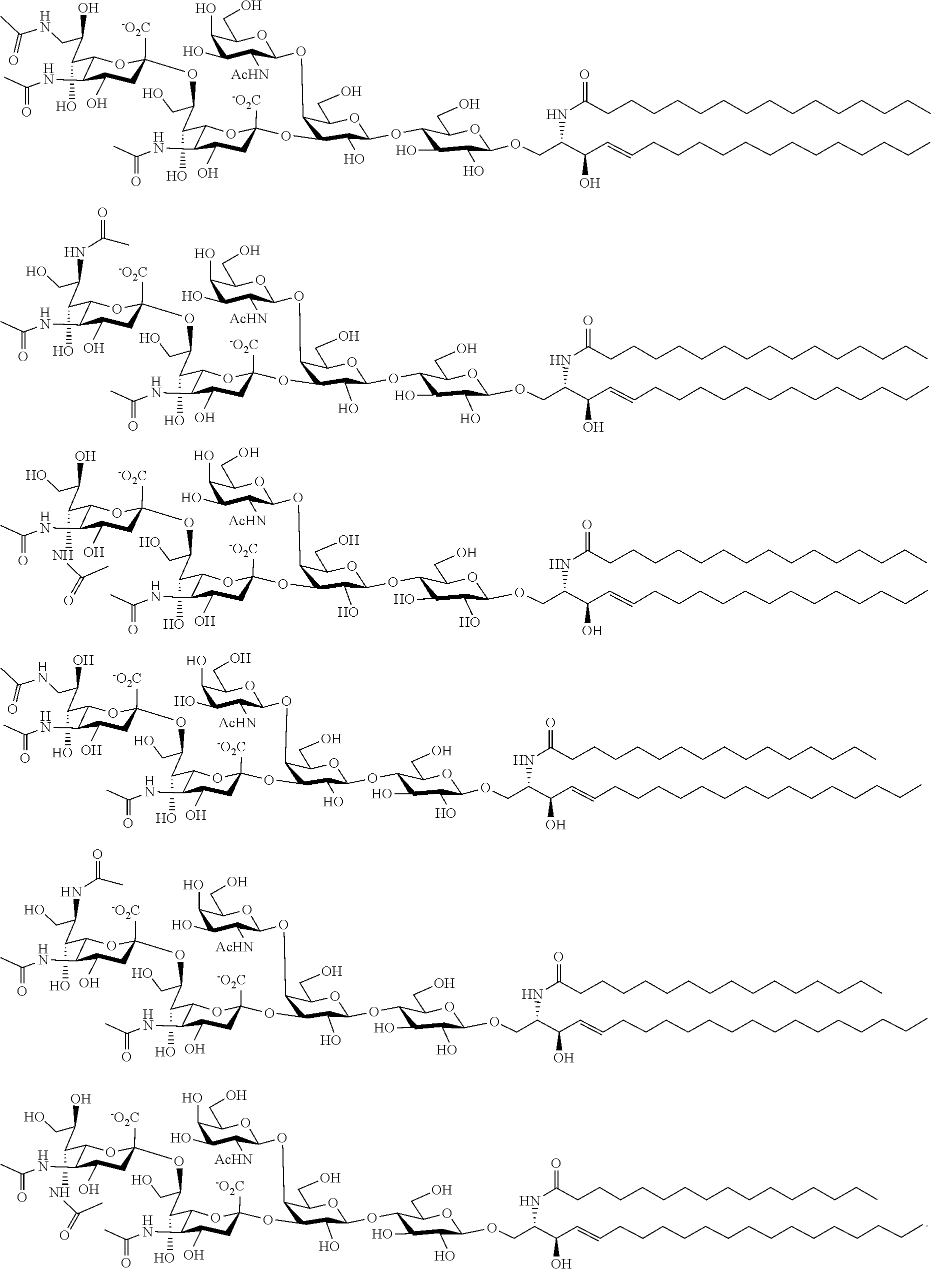

Optionally, the compound is a GD1b-analog selected from the group consisting of

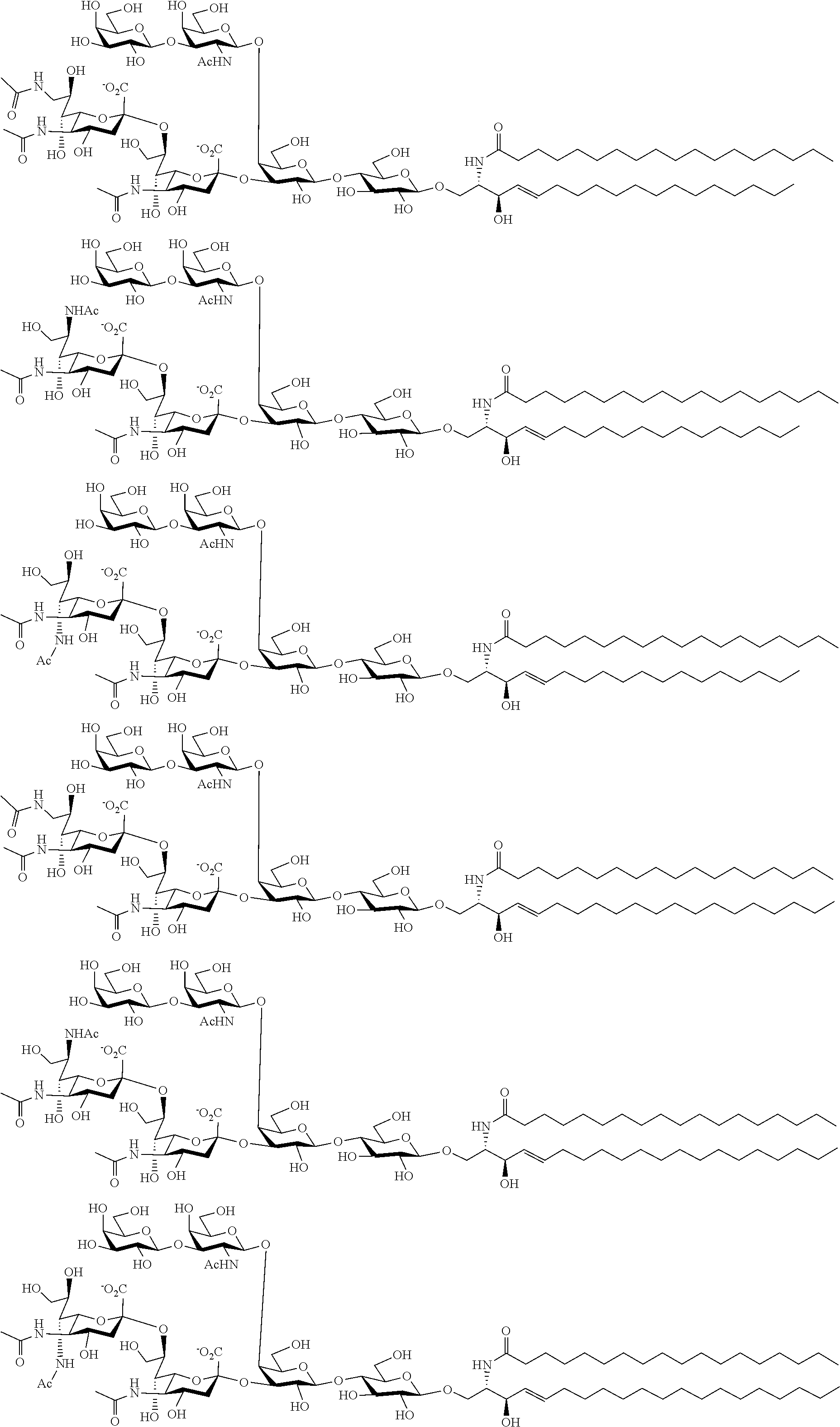

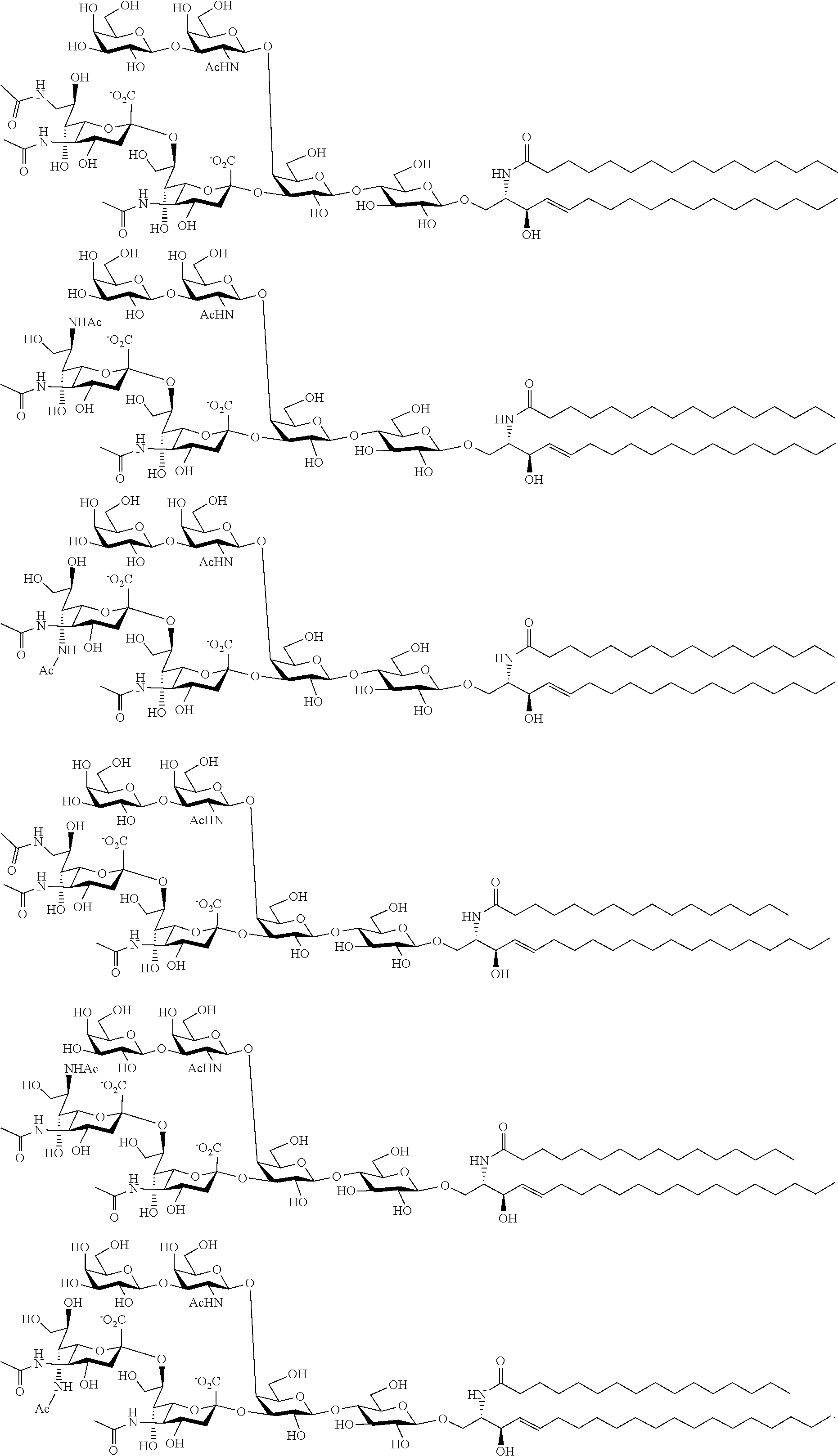

Optionally, the compound is a GT1b-analog selected from the group consisting of

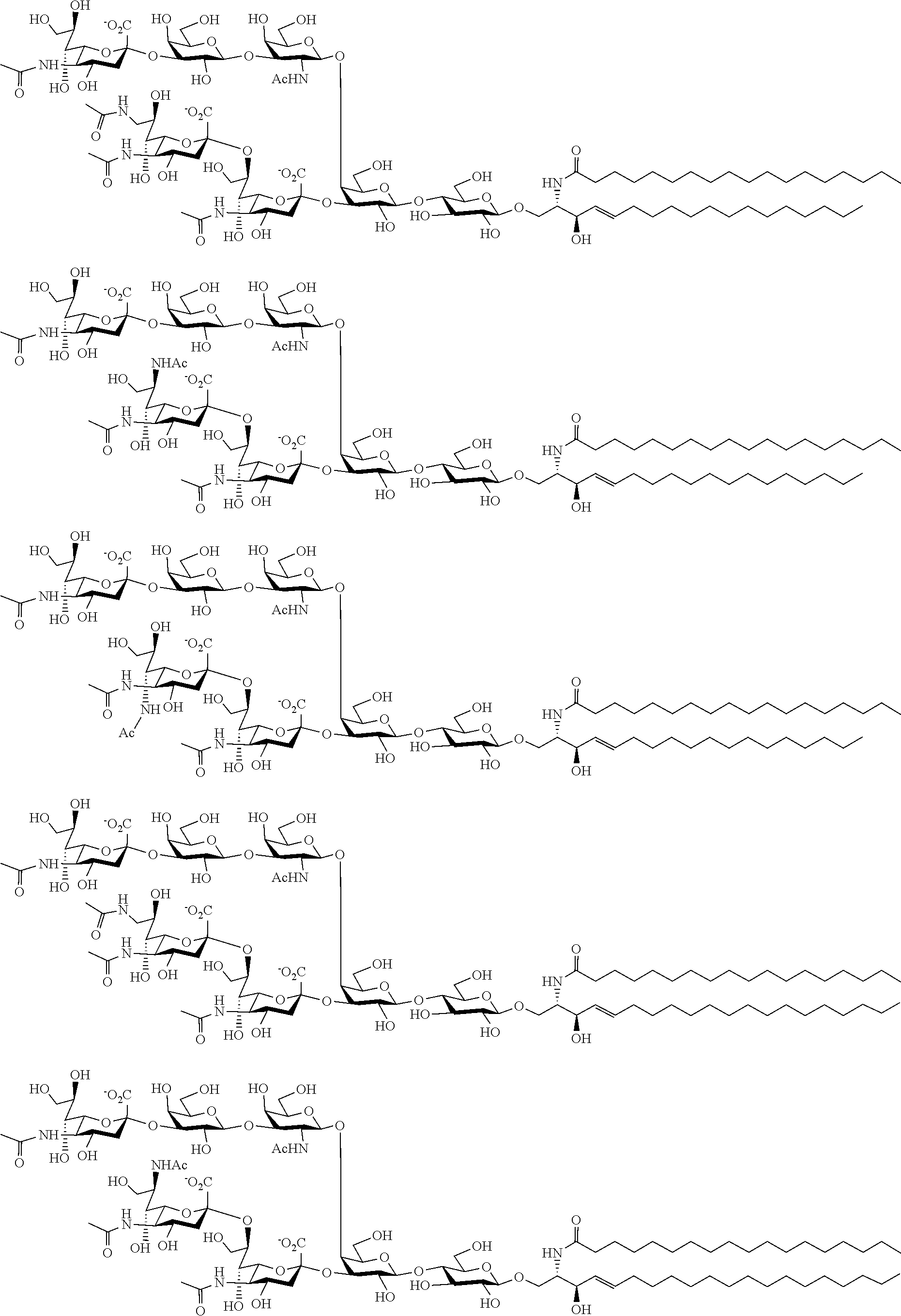

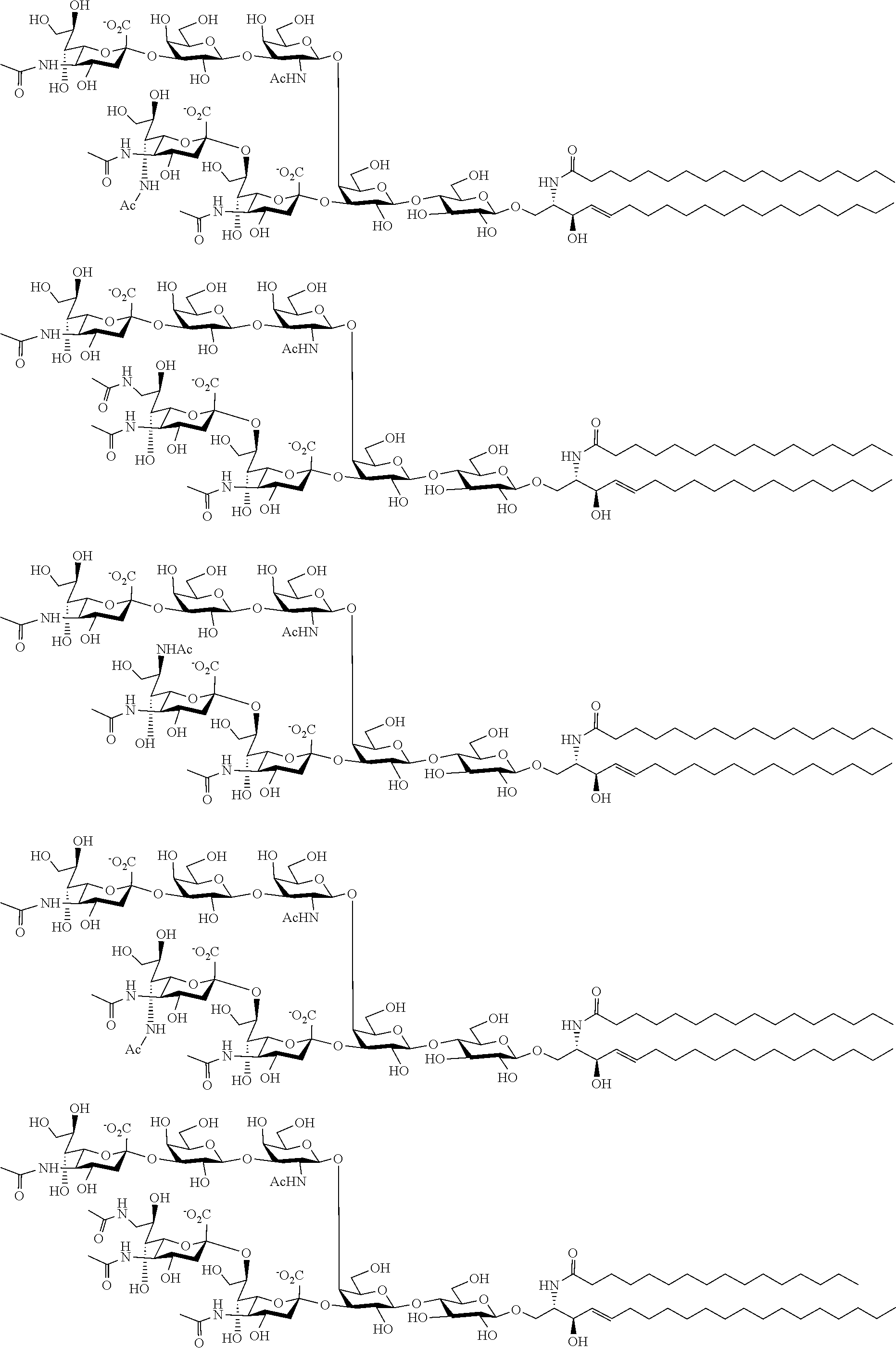

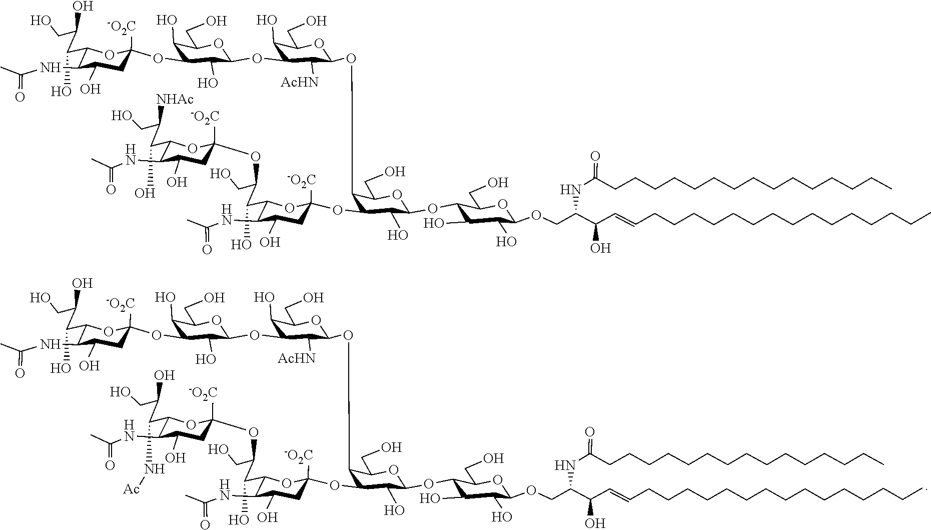

Optionally, the compound is a GQ1b-analog selected from the group consisting of

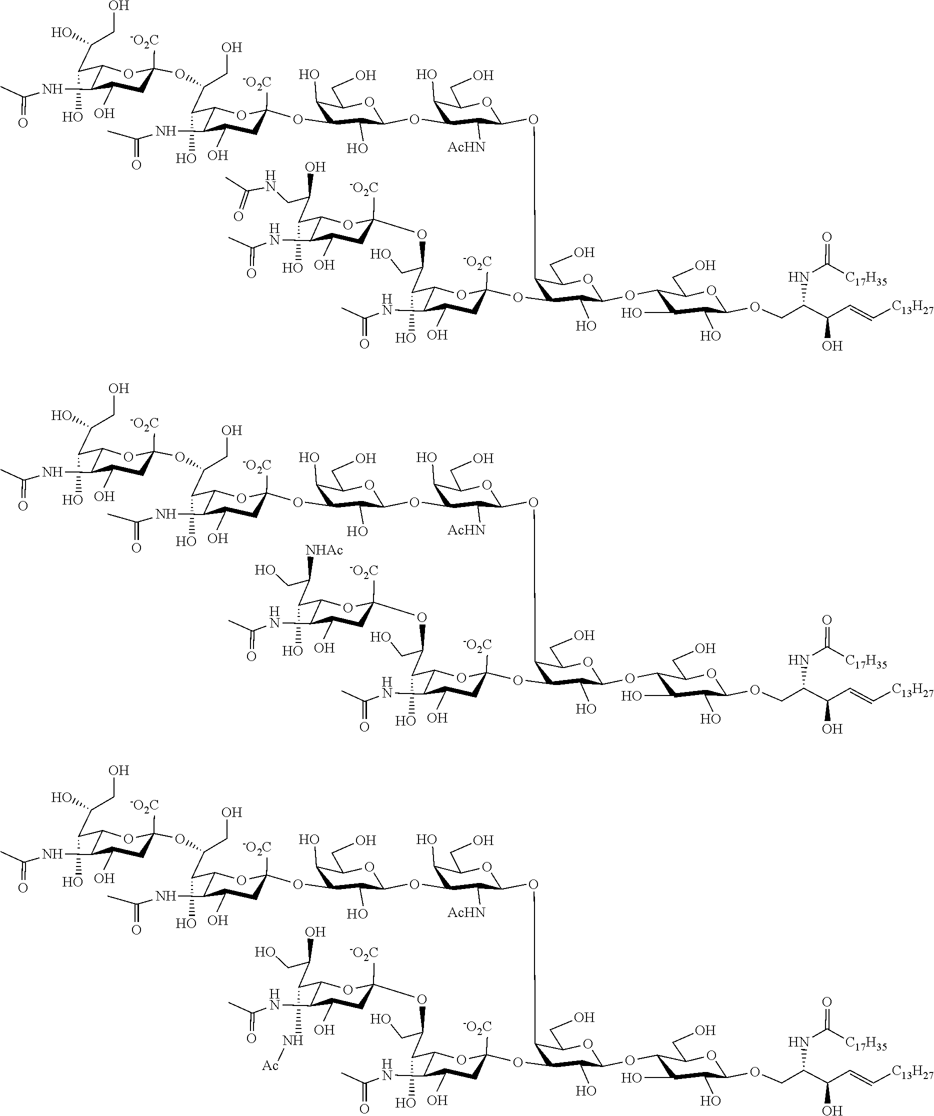

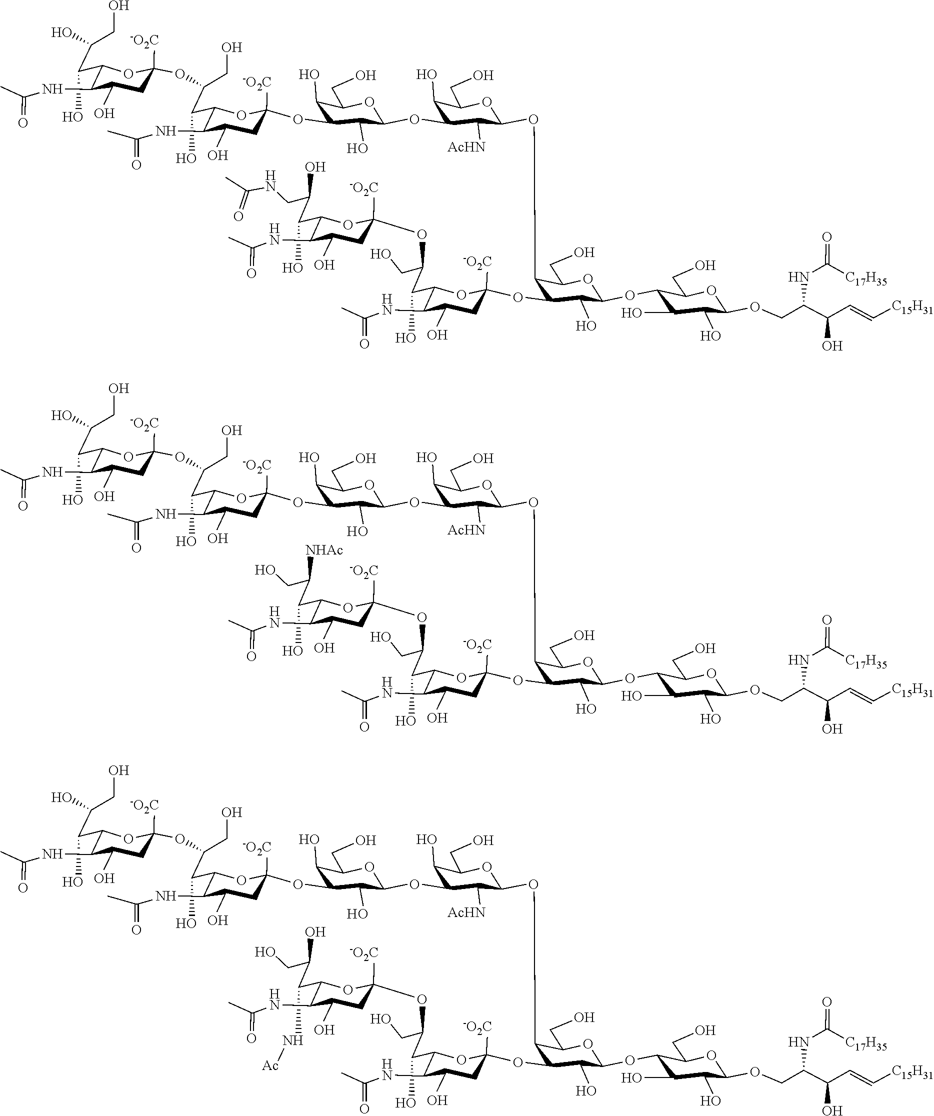

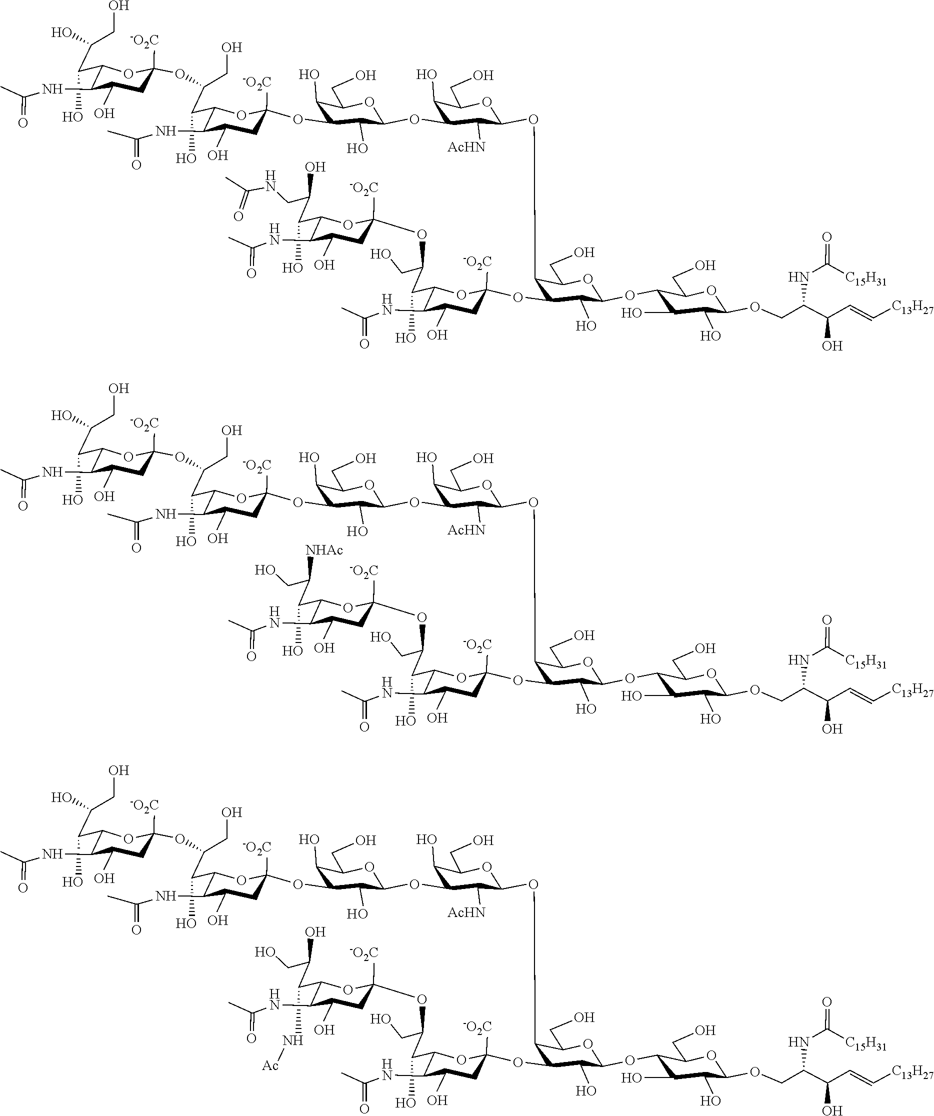

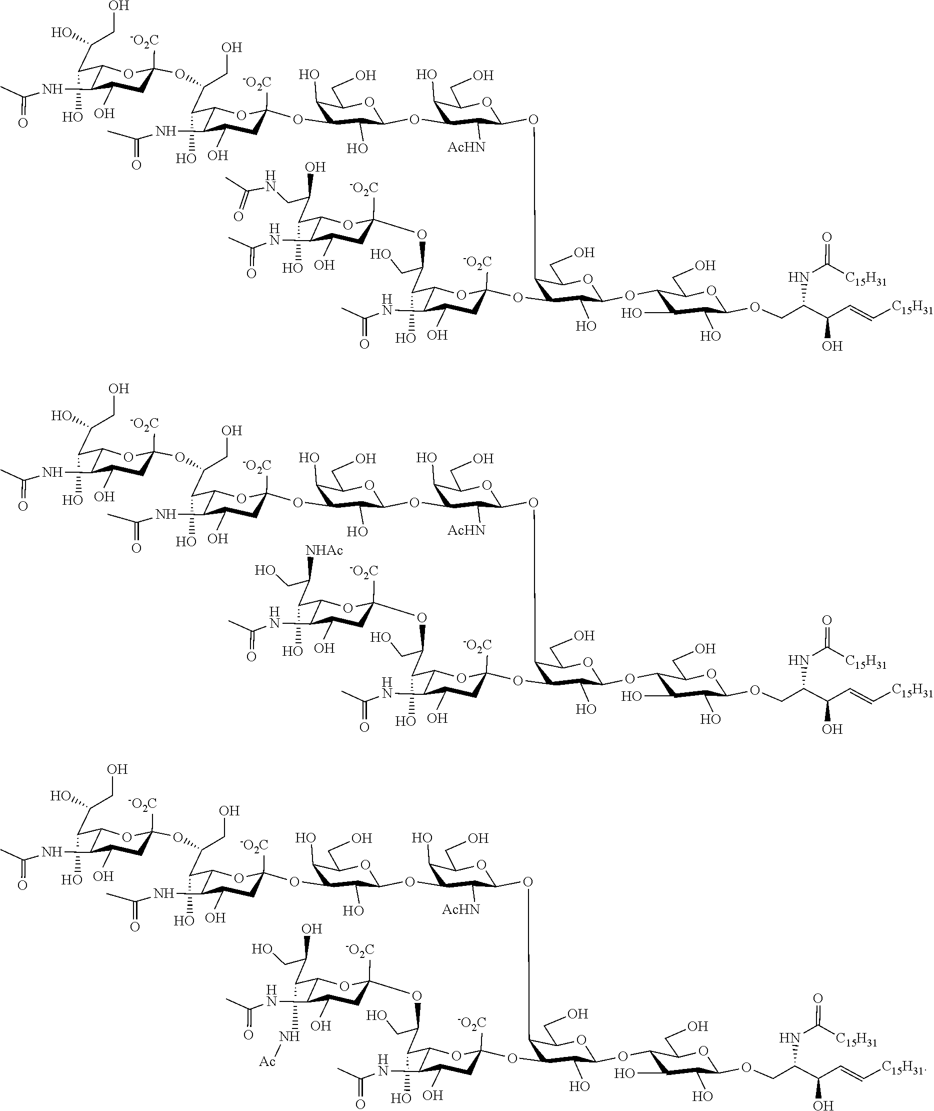

Optionally, the compound is a GD3 sphingosine selected from the group consisting of

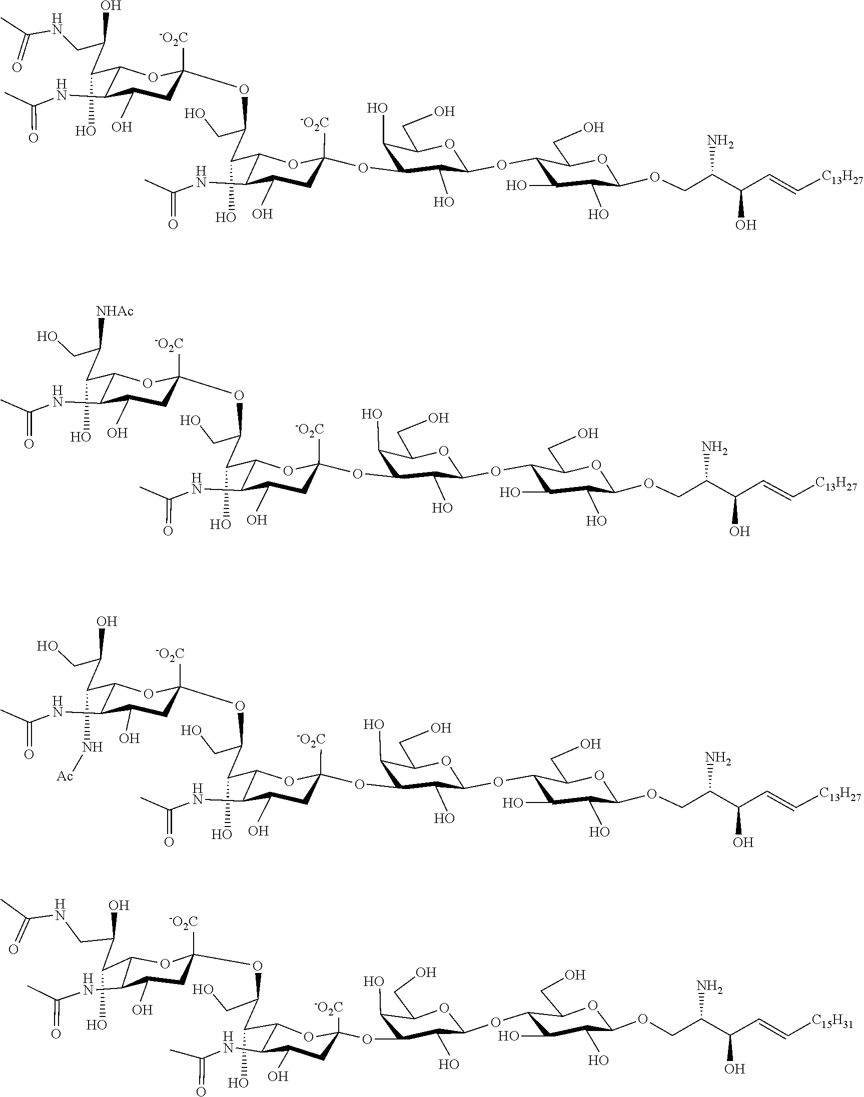

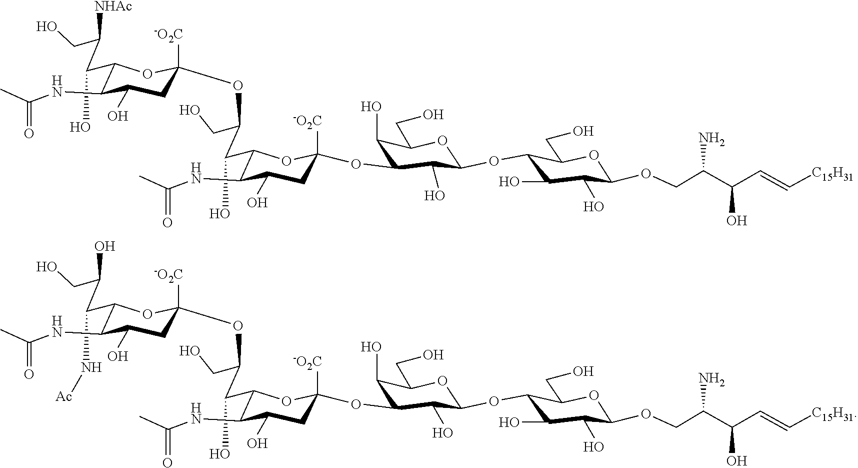

Optionally, the compound is a GD2 sphingosine selected from the group consisting of

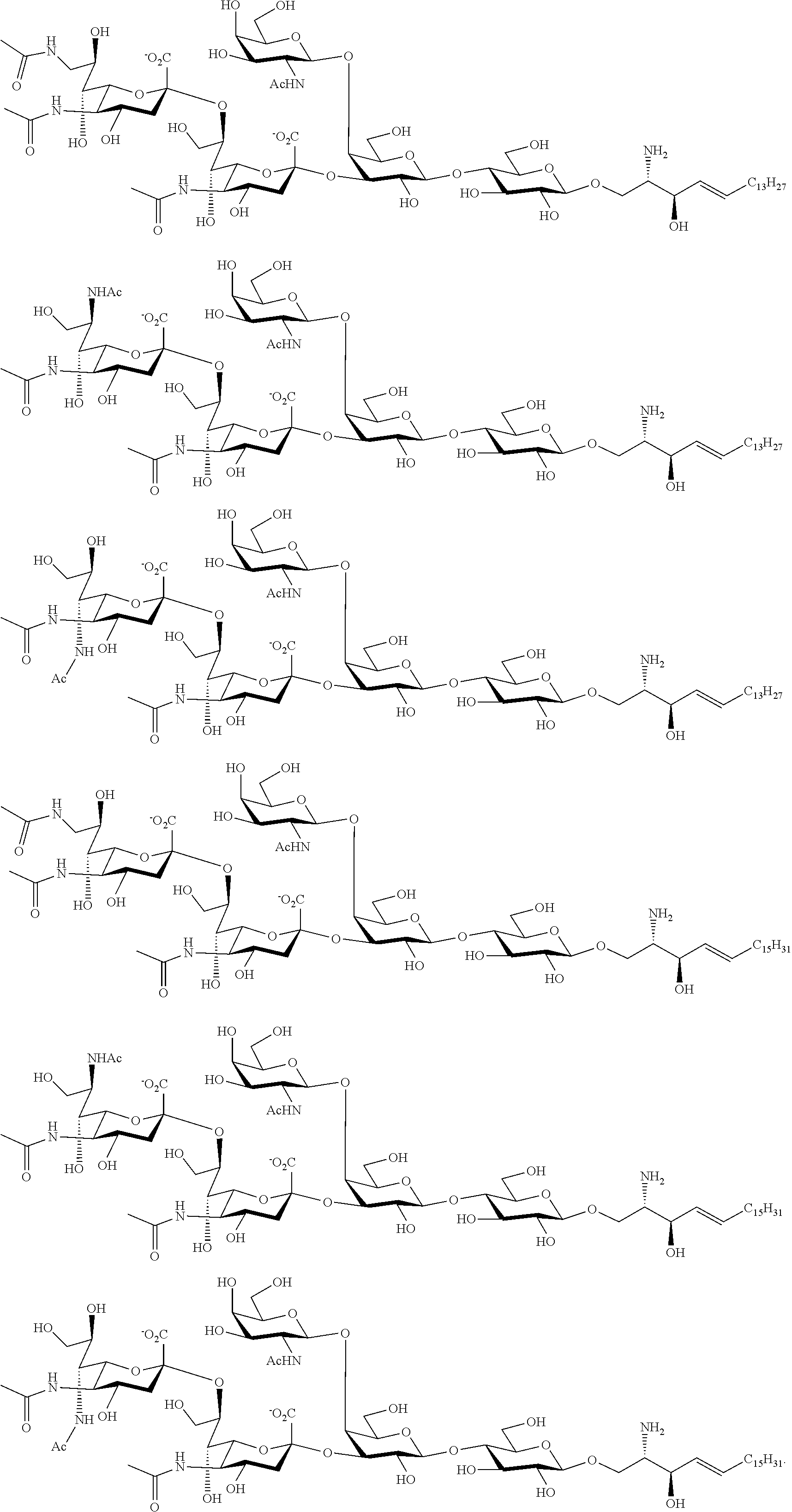

Optionally, the compound is a GD1b sphingosine selected from the group consisting of

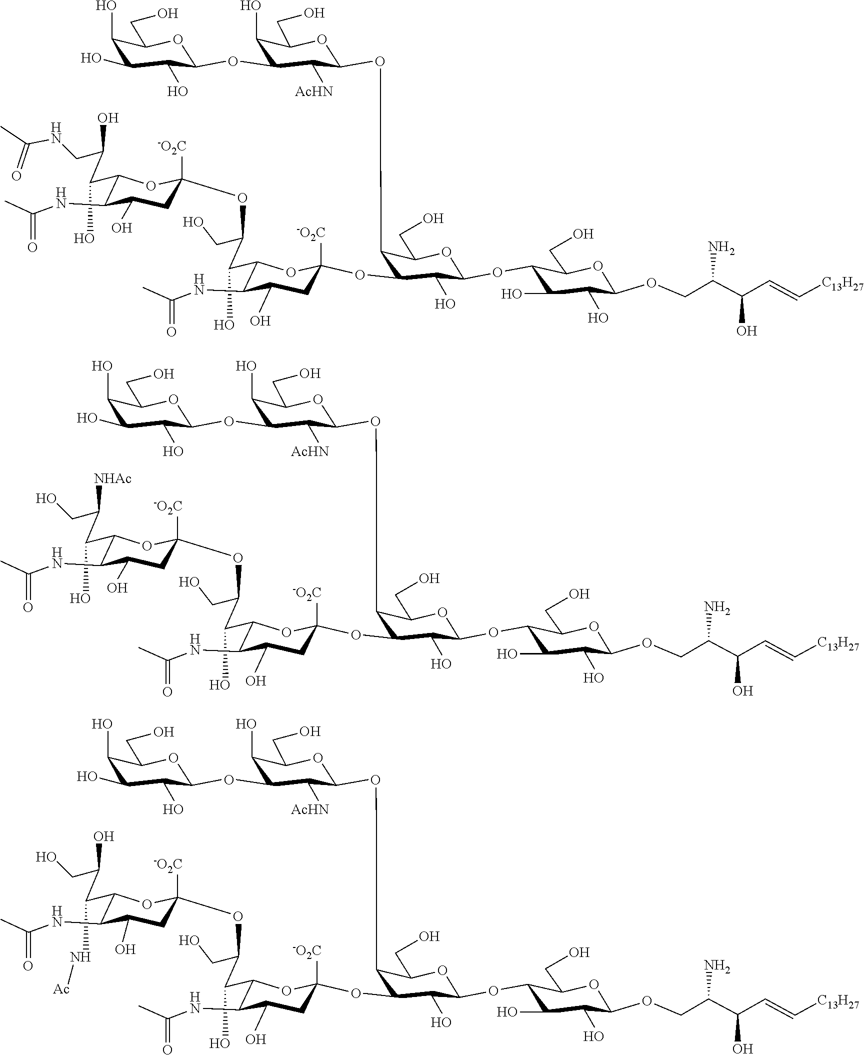

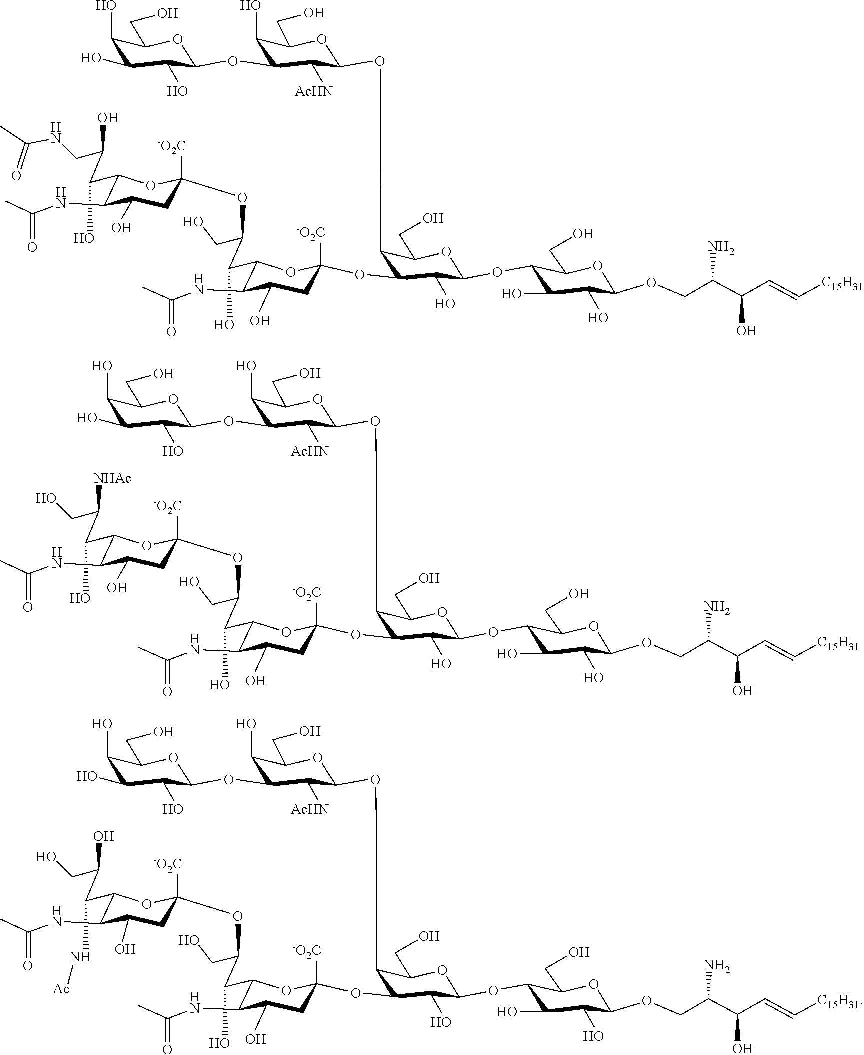

Optionally, the compound is a GT1b sphingosine selected from the group consisting of

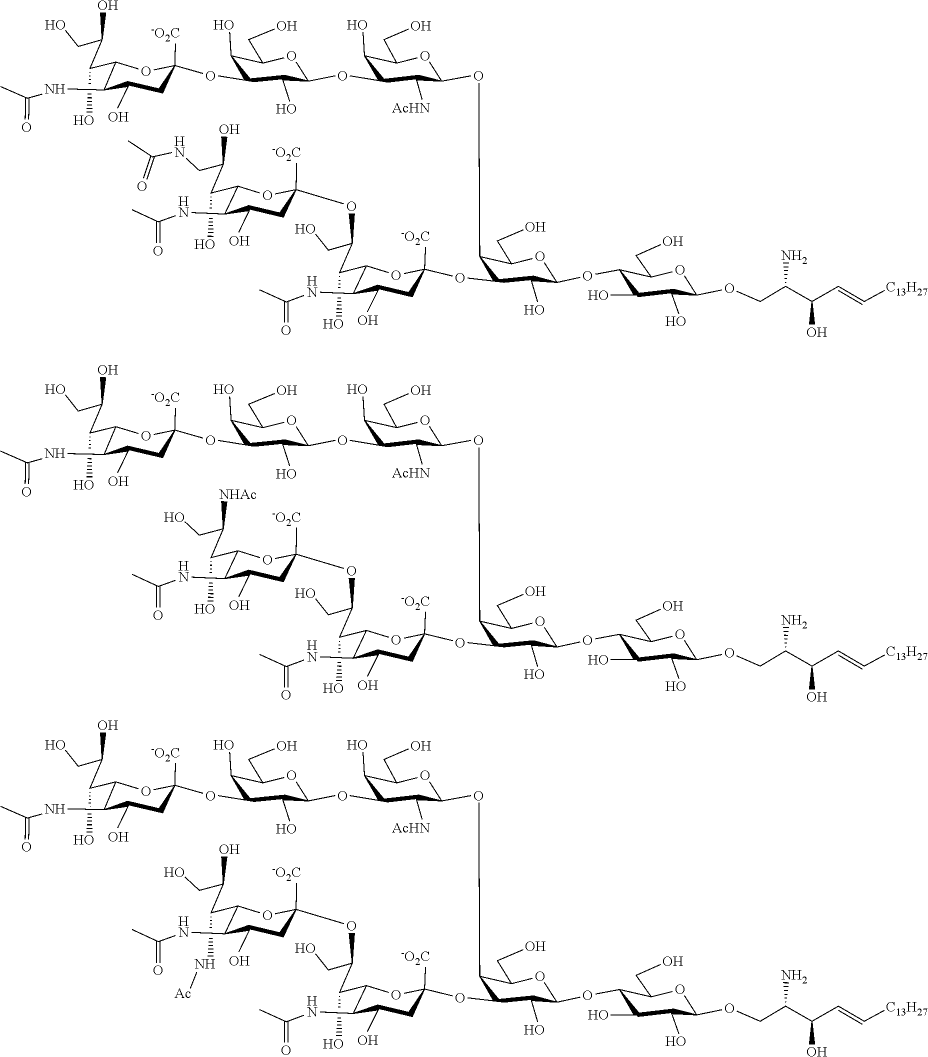

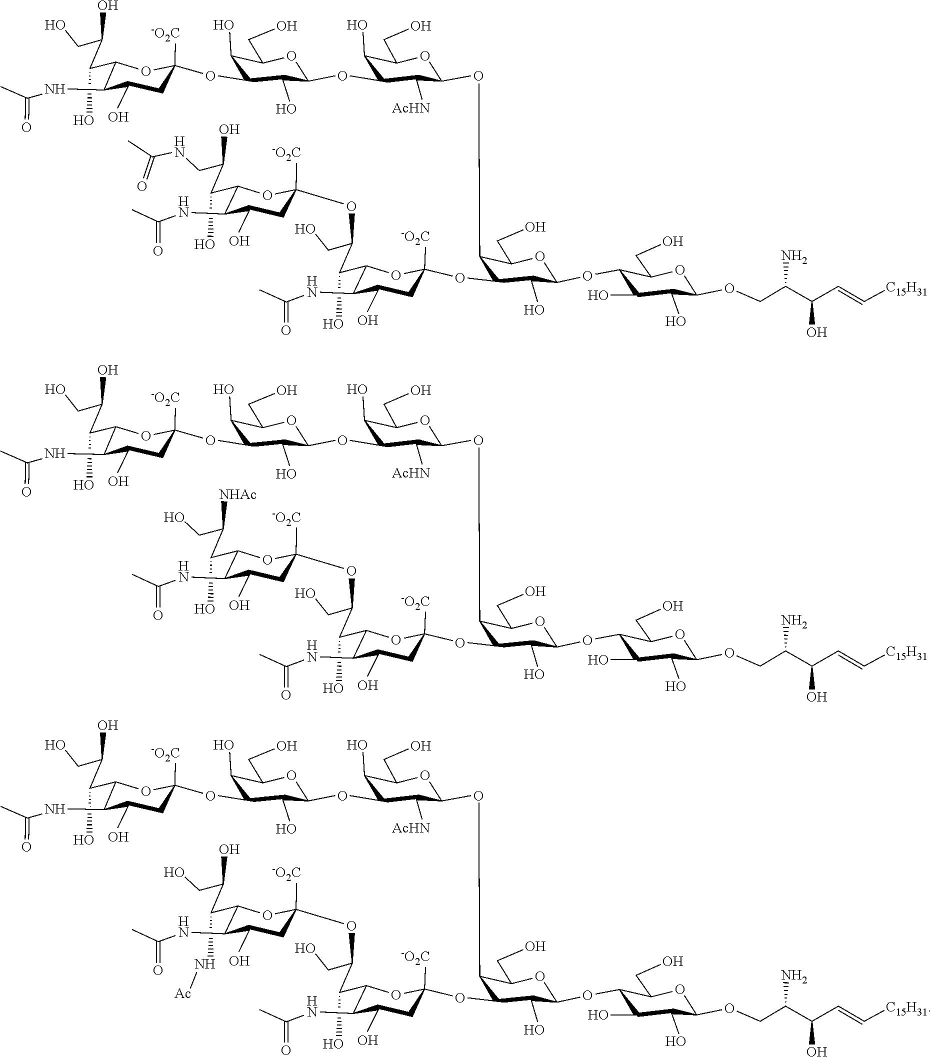

Optionally, the compound is a GQ1b sphingosine selected from the group consisting of

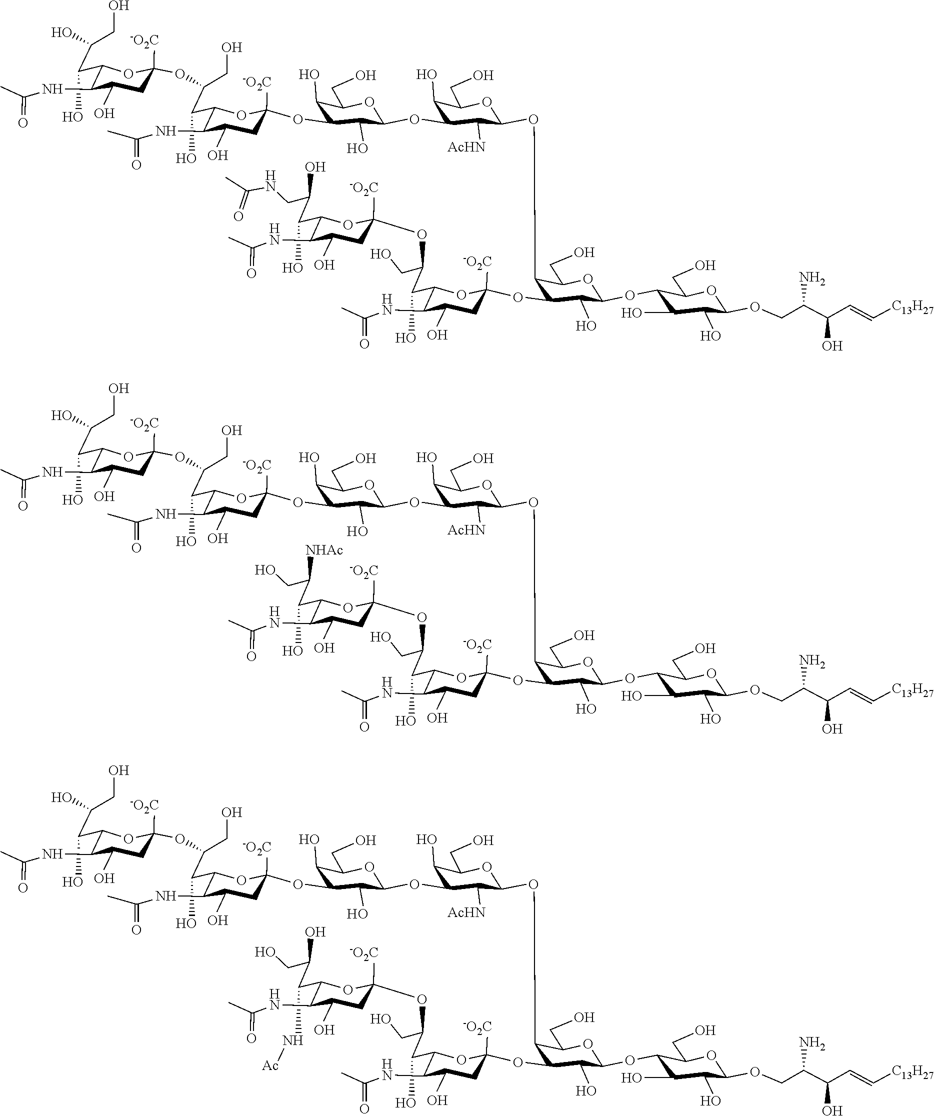

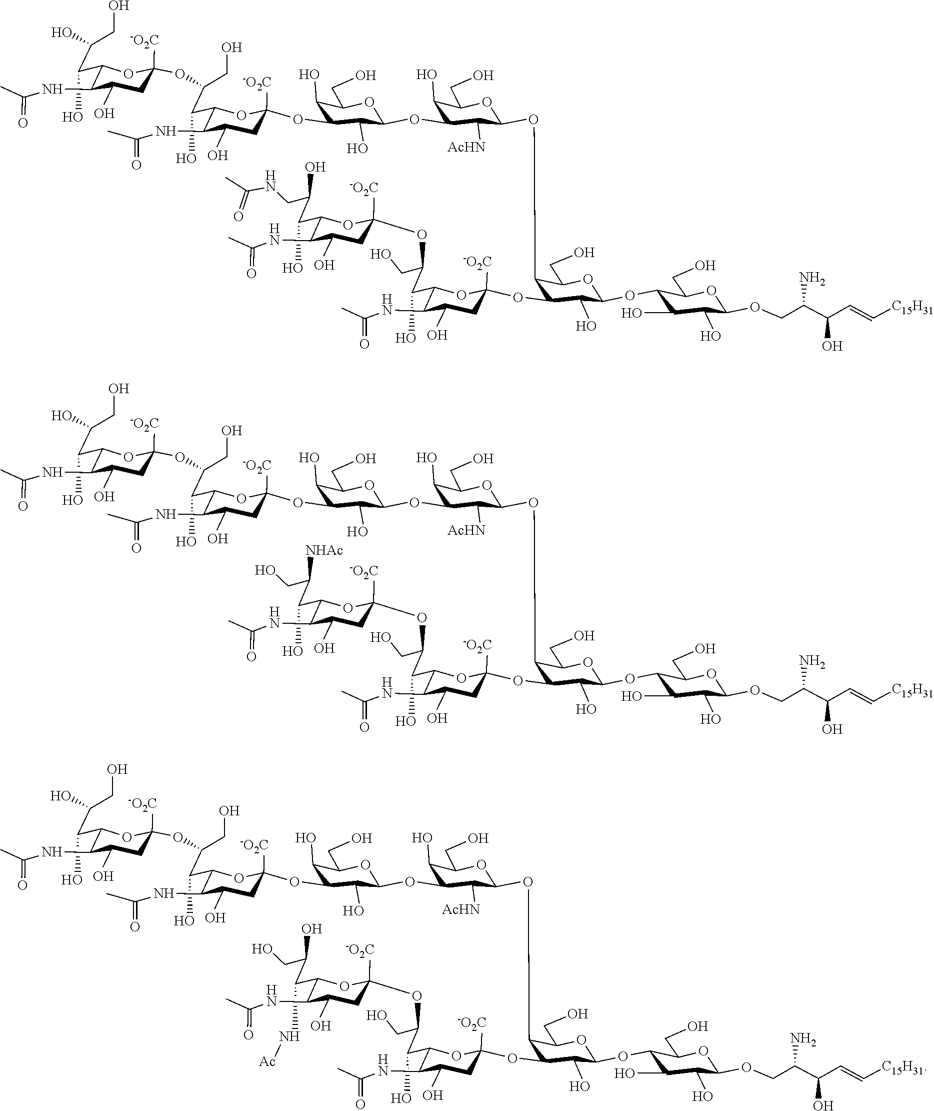

### Methods of Synthesis

Provided herein are methods of synthesizing N-acetyl-containing b-series gangliosides. The methods include a step (step (i)) of forming a reaction mixture comprising a donor precursor, cytidine 5′-triphosphate, pyruvate, a sialic acid aldolase, a CMP-sialic acid synthetase, a sialyltransferase, and an acceptor comprising an a-series sphingosine, under conditions sufficient to result in the sialylation of the a-series sphingosine to form a b-series sphingosine of the following formula:

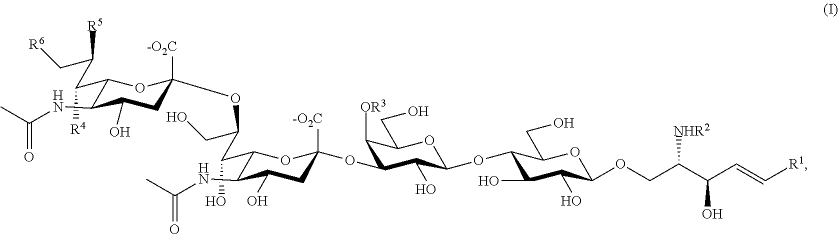

or a salt thereof, wherein R¹ is an alkyl chain; R² is H; R³ is H, a monosaccharide, or an oligosaccharide; and R⁴, R⁵, and R⁶ are each independently selected from OH, NHAc, or N₃, wherein at least one of R⁴, R⁵, and R⁶ is NHAc or N₃. The methods also include a step (step (ii)) of acetylating the b-series sphingosine to form a b-series ganglioside of Formula I:

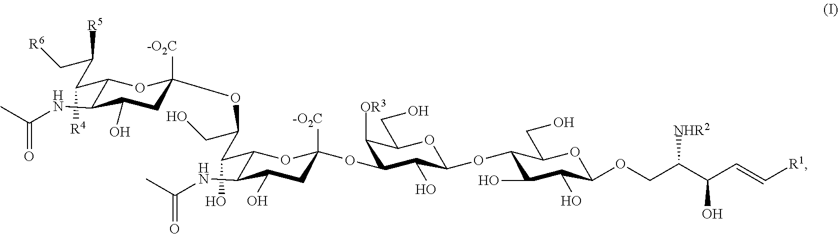

or a salt thereof, wherein R¹ is an alkyl chain; R² is —C(O)alkyl; R³ is H, a monosaccharide, or an oligosaccharide; R⁴, R⁵, and R⁶ are each independently selected from OH, NHAc, or N₃, wherein at least one of R⁴, R⁵, and R⁶ is NHAc or N₃.

The donor precursor can include an N-acetyl group or an azido group. Optionally, the donor precursor is 6-acetamido-6-deoxy-N-acetylmannosamine (ManNAc6NAc), 6-azido-6-deoxy-N-acetylmannosamine (ManNAc6N₃), 4-acetamido-4-deoxy-N-acetylmannosamine (ManNAc4NAc), 4-azido-4-deoxy-N-acetylmannosamine (ManNAc4N₃), 5-acetamido-5-deoxy-N-acetylmannosamine (ManNAc5NAc), or 5-azido-5-deoxy-N-acetylmannosamine (ManNAc5N₃).

The acceptor can include GM3βSph.

Optionally, the sialic acid aldolase is P. multocida sialic acid aldolase (PmAldolase) or Escherichia coli sialic acid aldolase (EcAldolase).

Any suitable CMP-sialic acid synthetase (i.e., N-acylneuraminate cytidylyltransferase, EC 2.7.7.43, also referred to as “CSS”) can be used in the methods for forming the sialosides disclosed herein. For example, CMP-sialic acid synthetases from E. coli, C. thermocellum, S. agalactiae, or N. meningitidis can be used. In some embodiments, the CMP sialic acid synthetase is Neisseria meningitidis CMP-sialic acid synthetase (NmCSS). In other embodiments, the CMP-sialic acid synthetase is Legionellapneumophila CMP-5,7-di-N-acetyllegionaminic acid synthetase (LpCLS).

In some embodiments, the CMP-sialic acid synthetase is NmCSS (NCBI Accession No. WP_025459740.1) or a catalytically active variant thereof. In some embodiments, the CMP-sialic acid synthetase comprises the polypeptide sequence: EKQNIAVILARQNSKGLPLKNLRKMNGISLLGHTINAAISSKCFDRIIVSTDGGLIAEEAK NFGVEVVLRPAELASDTASSISGVIHALETIGSNSGTVTLLQPTSPLRTGAHIREAFSLFDE KIKGSVVSACPMEHHPLKTLLQINNGEYAPMRHLSDLEQPRQQLPQAFRPNGAIYINDT ASLIANNCFFIAPTKLYIMSHQDSIDIDTELDLQQAENILTHHKES (SEQ ID NO:1), or a catalytically active variant thereof.

Any suitable sialyltransferase (also referred to as “ST”) can be used in the methods disclosed herein. In some embodiments, the sialyltransferase is a beta-galactoside alpha-2,3-sialyltransferase belonging to Glycosyltransferase family 42 (GT42 using CAZy nomenclature). Optionally, the sialyltransferase is a beta-galactoside alpha-2,8 sialyltransferase belonging to the GT42 CAZy family. Optionally, the sialyltransferase is a bifunctional enzyme having both alpha-2,3-sialyltransferase and alpha-2,8-sialyltransferase activities.

In some embodiments, the sialyltransferase is PmST3 or a catalytically active variant thereof, as described in U.S. Pat. No. 9,783,838, which is incorporated herein by reference in its entirety. In some embodiments, the sialyltransferase is PmST3A35 or a catalytically active variant thereof. In some embodiments, the sialyltransferase comprises the polypeptide sequence: DKFAEHEIPKAVIVAGNGESLSQIDYRLLPKNYDVFRCNQFYFEERYFLGNKIKAVFFTP GVFLEQYYTLYHLKRNNEYFVDNVILSSFNHPTVDLEKSQKIQALFIDVINGYEKYLSKL TAFDVYLRYKELYENQRITSGVYMCAVAIAMGYTDIYLTGIDFYQASEENYAFDNKKPN IIRLLPDFRKEKTLFSYHSKDIDLEAL SFLQQHYHVNFYSISPMSPL SKHFPIPTVEDDCET TFVAPLKENYINDILLVDKLAAALE (SEQ ID NO:2), or a catalytically active variant thereof.

In some embodiments, the sialyltransferase is Cst-II (GenBank Accession No. CS299360) or a catalytically active variant thereof. Cst-II are variants thereof are described by Cheng and Chen, et al. (Glycobiology, 18(9): 686-697, 2008), which is incorporated herein by reference in its entirety. In some embodiments, the sialyltransferase is C-His6-tagged Cst-II-Δ32¹⁵³s or a catalytically active variant thereof. In some embodiments, the sialyltransferase comprises the polypeptide sequence: KKVIIAGNGPSLKEIDYSRLPNDFDVFRCNQFYFEDKYYLGKKCKAVFYNPSLFFEQYYT LKHLIQNEYETELIMCSNYNQAHLENENFVKTFYDYFPDAHLGYDFFKQLKDFNAYFKF HEIYFNQRITSGVYMCAVAIALGYKEIYLSGIDFYQNGSSYAFDTKQKNLLKLAPNFKND NSHYIGHSKNTDIKALEFLEKTYKIKLYCLCPNSLLANFIELAPNLMSNFIIQEKNNYTKDI LIPSSEAYGKFSKNIN (SEQ ID NO:3), or a catalytically active variant thereof. In some cases, the sialyltransferase is Campylobacter jejuni α2-3/8-sialyltransferase II (CjCst-II).

In some cases, the sialyltransferase is Campylobacterjejuni α2-3-sialyltransferase I (CjCst-I) (MBP-CjCst-IΔ145-His6). CjCst-I is described by Zhang, et al. (Molecules, 28(6): 2753, 2023), which is incorporated herein by reference in its entirety. In some embodiments, the MBP-CjCst-IΔ145-His6includes the following DNA (a) and the amino acid (b) sequences.

The sequences from the vector are underlined. The linker and the His6-tag sequences are shown in bold.

(a)
(SEQ ID NO: 4)
ATGAAAATCGAAGAAGGTAAACTGGTAATCTGGATTAACGGCGATAAAGG
CTATAACGGTCTCGCTGAAGTCGGTAAGAAATTCGAGAAAGATACCGGAA
TTAAAGTCACCGTTGAGCATCCGGATAAACTGGAAGAGAAATTCCCACAG
GTTGCGGCAACTGGCGATGGCCCTGACATTATCTTCTGGGCACACGACCG
CTTTGGTGGCTACGCTCAATCTGGCCTGTTGGCTGAAATCACCCCGGACA
AAGCGTTCCAGGACAAGCTGTATCCGTTTACCTGGGATGCCGTACGTTAC
AACGGCAAGCTGATTGCTTACCCGATCGCTGTTGAAGCGTTATCGCTGAT
TTATAACAAAGATCTGCTGCCGAACCCGCCAAAAACCTGGGAAGAGATCC
CGGCGCTGGATAAAGAACTGAAAGCGAAAGGTAAGAGCGCGCTGATGTTC
AACCTGCAAGAACCGTACTTCACCTGGCCGCTGATTGCTGCTGACGGGGG
TTATGCGTTCAAGTATGAAAACGGCAAGTACGACATTAAAGACGTGGGCG
TGGATAACGCTGGCGCGAAAGCGGGTCTGACCTTCCTGGTTGACCTGATT
AAAAACAAACACATGAATGCAGACACCGATTACTCCATCGCAGAAGCTGC
CTTTAATAAAGGCGAAACAGCGATGACCATCAACGGCCCGTGGGCATGGT
CCAACATCGACACCAGCAAAGTGAATTATGGTGTAACGGTACTGCCGACC
TTCAAGGGTCAACCATCCAAACCGTTCGTTGGCGTGCTGAGCGCAGGTAT
TAACGCCGCCAGTCCGAACAAAGAGCTGGCAAAAGAGTTCCTCGAAAACT
ATCTGCTGACTGATGAAGGTCTGGAAGCGGTTAATAAAGACAAACCGCTG
GGTGCCGTAGCGCTGAAGTCTTACGAGGAAGAGTTGGCGAAAGATCCACG
TATTGCCGCCACTATGGAAAACGCCCAGAAAGGTGAAATCATGCCGAACA
TCCCGCAGATGTCCGCTTTCTGGTATGCCGTGCGTACTGCGGTGATCAAC
GCCGCCAGCGGTCGTCAGACTGTCGATGAAGCCCTGAAAGACGCGCAGAC
TAATTCGAGCTCGAACAACAACAACAATAACAATAACAACAACCTCGGGA
TCGAGGGAAGGATTTCAGAATTCATGACACGCACCAGAATGGAGAACGAG
TTGATTGTCTCAAAGAACATGCAAAACATCATTATCGCAGGCAATGGCCC
CAGCCTCAAGAATATTAATTACAAGCGTCTCCCGCGGGAATACGACGTTT
TCCGTTGTAACCAATTTTACTTCGAGGACAAGTATTACTTAGGGAAGAAG
ATAAAGGCAGTTTTCTTTAACCCTGGCGTATTCTTACAACAATACCACAC
CGCTAAGCAACTTATACTCAAGAATGAATACGAGATCAAGAACATTTTCT
GTTCCACTTTCAATCTCCCGTTTATTGAGAGCAACGATTTCCTGCACCAA
TTTTACAACTTCTTCCCCGATGCAAAACTGGGCTACGAGGTTATCGAGAA
CCTGAAGGAATTTTACGCCTACATCAAGTATAATGAGATATACTTTAACA
AGCGTATCACATCCGGGGTATATATGTGTGCCATCGCCATAGCCCTGGGT
TACAAGACCATTTACTTGTGCGGAATTGACTTCTACGAGGGCGATGTGAT
CTATCCATTCGAGGCTATGTCAACTAATATTAAGACCATCTTCCCGGGCA
TTAAAGACTTTAAGCCGAGCAATTGTCATAGTAAAGAGTACGACATCGAA
GCATTAAAACTTCTGAAATCGATATACAAGGTGAATATATACGCGCTCTG
TGACGACTCCATATTAGCAAACCATTTCCCTTTGAGCATCAACATCAATA
ACAATTTCACATTGGAGAACAAGCACAACAATAGTATCAACGACATCCTG
TTAACTGACAACACACCGGGTGTTTCATTTTACAAGAATCAACTGAAAGC
AGATAACAAGATAATGCTGAACTTTTATCATCATCATCATCATCATTAA
(b)
(SEQ ID NO: 5)
MKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRY
NGKLIAYPIAVEALSLIYNKDLLPNPPKTWEEIPALDKELKAKGKSALME
NLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPT
FKGQPSKPFVGVLSAGINAASPNKELAKEFLENYLLTDEGLEAVNKDKPL
GAVALKSYEEELAKDPRIAATMENAQKGEIMPNIPQMSAFWYAVRTAVIN
AASGRQTVDEALKDAQTNSSSNNNNNNNNNNLGIEGRISEFMTRTRMENE
LIVSKNMQNIIIAGNGPSLKNINYKRLPREYDVFRCNQFYFEDKYYLGKK
IKAVFFNPGVFLQQYHTAKQLILKNEYEIKNIFCSTFNLPFIESNDFLHQ
FYNFFPDAKLGYEVIENLKEFYAYIKYNEIYENKRITSGVYMCAIAIALG
YKTIYLCGIDFYEGDVIYPFEAMSTNIKTIFPGIKDFKPSNCHSKEYDIE
ALKLLKSIYKVNIYALCDDSILANHFPLSININNNFTLENKHNNSINDIL
LTDNTPGVSFYKNOLKADNKIMLNFYHHHHHH

The method described herein can further include one or more glycosylation steps following step (i). Such steps are detailed in exemplary methods provided in the Examples section herein.

Optionally, an azido group can be present in the compound or an intermediate, and the azido can be further reduced to an amino group. For example, when the donor precursor is 6-azido-6-deoxy-N-acetylmannosamine (ManNAc6N₃), 4-azido-4-deoxy-N-acetylmannosamine (ManNAc4N₃) or 5-azido-5-deoxy-N-acetylmannosamine (ManNAc5N₃), the method can further include a step of reducing the azido group to an amino group after step (ii).

Optionally, an azido group can be present in the compound or an intermediate, and the azido can be further converted to an acetamido group. For example, when the donor precursor is 6-azido-6-deoxy-N-acetylmannosamine (ManNAc6N₃), 4-azido-4-deoxy-N-acetylmannosamine (ManNAc4N₃) or 5-azido-5-deoxy-N-acetylmannosamine (ManNAc5N₃), the method can further include a step of converting the azido group to an acetamido group after step (ii).

Methods for preparing the compounds described herein generally include providing reaction mixtures that contain the one or more enzymes as described herein. An enzyme for use in the methods described herein can be, for example, isolated or otherwise purified prior to addition to the reaction mixture. As used herein, a “purified” enzyme refers to an enzyme which is provided as a purified protein composition wherein the enzyme constitutes at least about 50% of the total protein in the purified protein composition. For example, the enzyme can constitute about 50%, 60%, 70%, 75%, 80%, 85%, 90%, 95%, or 99% of the total protein in the purified protein composition. In some embodiments, the enzymes in the reaction mixture are provided as purified protein compositions wherein the enzyme constitutes at least about 95% of the total protein in purified protein composition (prior to addition to the reaction mixture). The amount of the enzyme in a purified protein composition can be determined by any number of known methods including, for example, by polyacrylamide gel electrophoresis (e.g., SDS-PAGE) followed by detection with a staining reagent (e.g., Coomassie Brilliant Blue G-250 and/or a silver nitrate stain). The enzymes used in the methods for forming sialosides can also be secreted by a cell present in the reaction mixture. Alternatively, the enzymes can catalyze the reaction within a cell expressing the variant.

Reaction mixtures can contain additional reagents for use in glycosylation techniques. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂), and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N,N-tetraacetic acid (EGTA), 2-({2-[bis(carboxymethyl)amino]ethyl}(carboxymethyl)amino)acetic acid (EDTA), and 1,2-bis(o-aminophenoxy)ethane-N,N,N,N-tetraacetic acid (BAPTA)), reducing agents (e.g., dithiothreitol (DTT), β-mercaptoethanol (BME), and tris(2-carboxyethyl)phosphine (TCEP)), and labels (e.g., fluorophores, radiolabels, and spin labels). Buffers, cosolvents, salts, detergents/surfactants, chelators, reducing agents, and labels can be used at any suitable concentration, which can be readily determined by one of skill in the art. In general, buffers, cosolvents, salts, detergents/surfactants, chelators, reducing agents, and labels are included in reaction mixtures at concentrations ranging from about 1 μM to about 1 M. For example, a buffer, a cosolvent, a salt, a detergent/surfactant, a chelator, a reducing agent, or a label can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M.

The enzymatic reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods of the invention, depending on the identity of a particular enzymes or acceptor molecules employed.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Chemoenzymatic Synthesis of N-Acetyl Analogues of 9-O-Acetylated b-Series Gangliosides

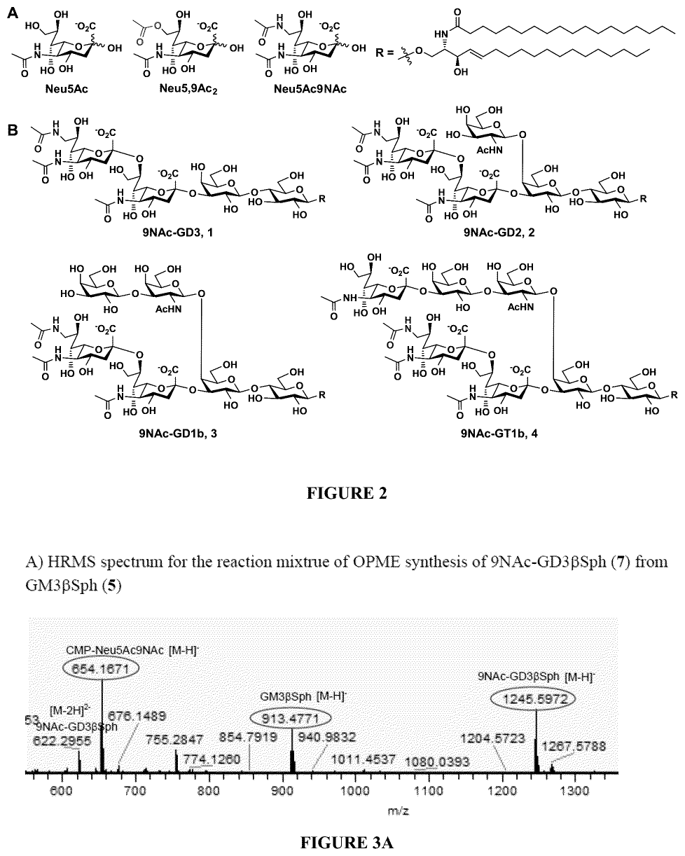

O-Acetylated gangliosides have been underutilized in drug development to date due to the instability of Sia 0-acetyl group towards pH change and/or esterase activities. To address the instability issue of the O-acetylated Sias and sialosides, a chemical biology strategy was previously used by substituting the oxygen atom in the ester group of Neu5,9Ac₂ with a nitrogen atom to generate a stable analogue 9-acetamido-9-deoxy-N-acetylneuraminic acid (Neu5Ac9NAc) (FIG. 2, Panel A). Such approaches suffered due to inefficiencies in methods, which would hinder their use in drug development.

Described herein is the development of efficient approaches for synthesizing Neu5Ac9NAc-containing b-series gangliosides including 9NAc-GD3, 9NAc-GD2, 9NAc-GD1b, and 9NAc-GT1b (FIG. 2, Panel B). Two chemoenzymatic methods have been developed for the synthesis of 9NAc-GD3 using either 6-azido-6-deoxy-N-acetylmannosamine (ManNAc6N₃) as a chemoenzymatic synthon or 6-acetamido-6-deoxy-N-acetylmannosamine (ManNAc6NAc) as an enzymatic precursor for 9-acetamido-9-deoxy-N-acetylneuraminic acid (Neu5Ac9NAc). In some cases, the latter method was found to be more efficient and was used to produce 9NAc-GD3, 9NAc-GD2, 9NAc-GD1b, and 9NAc-GT1b glycosylsphingosines. Furthermore, glycosylsphingosine acylation reaction conditions were improved to obtain target gangliosides in a faster reaction with an easier purification process compared to the previous acylation conditions. The resulting 9NAc-gangliosides can serve as stable analogs of 9-O-acetylated b-series gangliosides enabling studies to advance the understanding of the biological roles of ganglioside O-acetylation.

An efficient chemoenzymatic approach was developed for synthesizing 9-N-acetylated b-series gangliosides 9NAc-GD3 (1), 9NAc-GD2 (2), 9NAc-GD1b (3), and 9NAc-GT1b (4) from GM3 sphingosine (GM3βSph, 5) with ManNAc6NAc as the donor precursor for Neu5Ac9NAc. The 9-N-acetylated b-series gangliosides are stable analogues of the corresponding labile 9-O-acetylated counterparts which play important biological and pathological roles. The chemoenzymatic strategy reported here can be generally applied to access N-acetyl analogues of other gangliosides or glycans containing O-acetyl sialic acids.

Materials D-Galactose (Gal) was purchased from Fisher Scientific (Hampton, New Hampshire, USA). D-GalNAc was from Carbosynth US. Neu5Ac was bought from Ningbo Hongxiang Bio-chem Co., Ltd. (Ningbo, China). UTP, CTP, and ATP were from Hangzhou Meiya Pharmacy (Hangzhou, China). Recombinant enzymes Pasteurella multocida sialic acid aldolase (PmAldolase), Neisseria meningitidis CMP-sialic acid synthetase (NmCSS), Campylobacter jejuni multifunctional α2-3/8-sialyltransferase (CjCst-II), Bifidobacterium longum strain ATCC55813 N-acetylhexosamine-1-kinase (BLNahK), Pasteurella multocida N-acetylglucosamine uridylyltransferase (PmGlmU), Pasteurella multocida inorganic pyrophosphatase (PmPpA), Campylobacter jejuni β(1-4-N-acetylgalactosaminyltransferase MBP-A15CjCgtA-His6, Streptococcuspneumoniae TIGR4 galactokinase (SpGalK), Bifidobacterium longum UDP-sugar pyrophosphorylase (BLUSP), Campylobacter jejuni β1-3-galactosyltransferase MBP-CjCgtBΔ30-His6, and Campylobacter jejuni α2-3-sialyltransferase MBP-CjCst-IΔ145-His6 were expressed and purified.

### I. Synthesis of 9NAc-GD3 (I) from GM3βSph (5) via a 9N₃-GD3 (6) intermediate using ManNAc6N₃ as a chemoenzymatic synthon in a one-pot multienzyme (OPME) sialylation system following by chemical acylation and N₃ to NHAc conversion

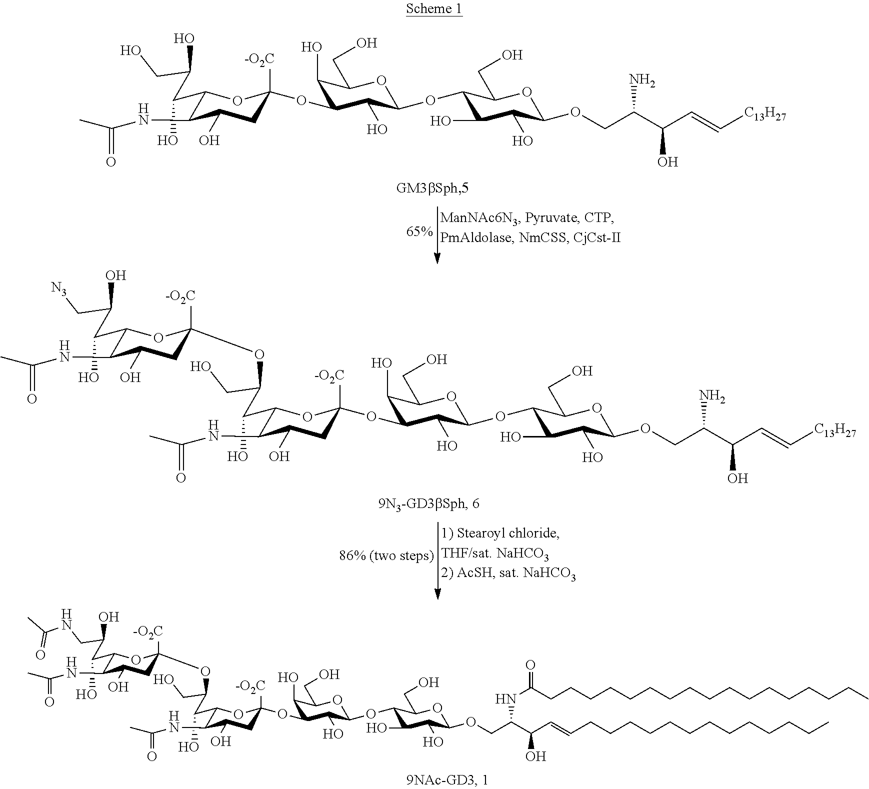

Synthesis of 9NAc-GD3 (6) using 6-azido-6-deoxy-N-acetylmannosamine (ManNAc6N₃) as a chemoenzymatic synthon: The synthesis of 9NAc-GD3 (1) was achieved from GM3 sphingosine (GM3βSph, 5) via a 9-azido-GD3 (9N₃-GD3, 6) intermediate using 6-azido-6-deoxy-N-acetylmannosamine (ManNAc6N₃) as a chemoenzymatic synthon. As shown in Scheme 1, 9N₃-GD3 glycosylsphingosine (9N₃-GD3βSph, 6) was produced from ManNAc6N₃ and GM3βSph (5) in the presence of sodium pyruvate and cytidine 5′-triphosphate (CTP) in an efficient one-pot multienzyme (OPME) sialylation system containing Pasteurella multocida sialic acid aldolase (PmAldolase), Neisseria meningitidis CMP-sialic acid synthetase (NmCSS), and Campylobacter jejuni α2-3/8-sialyltransferase (CjCst-II). OPME synthesis of 9NM-GD3βSph (5): (5-Acetamido-9-azido-3,5,9-trideoxy-D-glycero-α-D-galacto-2-nonulopyranosylonic acid)-(2→8)-(5-acetamido-3,5-dideoxy-D-glycero-α-D-galacto-2-nonulopyranosylonic acid)-(2→3)-3-D-galactopyranosyl-(1→4)-βD-glucopyranosyl-(1→1)-(2S, 3R, 4E)-2-amino-4-octadecene-1,3-diol: A reaction mixture in Tris-HCl buffer (100 mM, pH 8.5) in a total volume of 5 mL containing GM3βSph (5) (30 mg, 0.032 mmol), ManNAc6N₃ (16 mg, 0.065 mmol), sodium pyruvate (25 mg, 0.227 mmol), CTP (37 mg, 0.070 mmol), MgCl₂ (20 mM), PmAldolase (1.5 mg), NmCSS (0.8 mg), and CjCst-II (2 mg) was incubated in an incubator shaker at 30° C. with agitation at 100 revolutions per minute (rpm). The product formation was monitored by HRMS. After 24 hours, the reaction mixture was incubated in a boiling water bath for 5 minutes and then centrifuged to remove precipitates. The supernatant was concentrated and the residue was purified via a preconditioned Discovery DSC-18 SPE cartridge (bed wt. 10 g). After the sample was loaded, the C18 cartridge was washed with water (30 mL), and the 9N₃-GD3βSph was eluted with 40% acetonitrile in water (v/v). The unreacted GM3βSph was eluted with 60% acetonitrile in water (v/v). The fractions containing the pure product were collected, concentrated, and lyophilized to produce the desired pure 9N₃-GD3βSph (6) (27 mg, 65% yield). ¹H NMR (600 MHz, CD₃OD) δ 5.75 (dt, J=14.1, 6.9 Hz, 1H), 5.49 (dd, J=15.4, 7.6 Hz, 1H), 4.48 (d, J=7.9 Hz, 1H), 4.34 (d, J=7.8 Hz, 1H), 4.19-3.33 (m, 40H), 3.04-2.64 (m, 3H), 2.09 (q, J=7.4 Hz, 2H), 2.03 (d, J=2.2 Hz, 8H), 1.73 (q, J=11.9, 11.1 Hz, 2H), 1.30 (d, J=14.8 Hz, 31H), 0.90 (t, J=7.0 Hz, 3H). ¹³C NMR (150 MHz, CD₃OD) δ 174.16, 173.57, 173.29, 134.95, 134.30, 129.52, 103.35, 102.64, 101.58, 99.98, 79.26, 75.42, 75.31, 75.05, 74.57, 73.72, 73.35, 73.27, 73.11, 72.43, 70.77, 70.48, 69.62, 69.46, 68.10, 67.94, 67.10, 63.00, 61.88, 61.41, 54.79, 53.44, 52.73, 52.49, 41.51, 41.16, 32.06, 31.67, 29.39, 29.36, 29.28, 29.24, 29.07, 29.04, 28.99, 28.93, 22.34, 21.64, 21.26, 13.07. HRMS (ESI-Orbitrap) m/z: [M−H]⁻ calculated for C₅₂H₈₉N₆O₂₇ 1229.5781; found 1229.5776.

Conversion of 9N₃-GD3βSph (6) to 9NAc-GD3 (1): To a solution of 9N₃-GD3βSph (6) (15 mg) in saturated (sat.) NaHCO₃-THF (2 mL, 2:1), stearoyl chloride (1.5 equivalents (eq)) in 0.5 mL of THE was added. The resulting mixture was stirred vigorously at room temperature for 2 h. An additional amount of stearoyl chloride (1.0 eq) in THF was added and the reaction was stirred for another 1.5 hours. The product was purified via a preconditioned Discovery DSC-18 SPE cartridge (bed wt. 10 g). After the sample was loaded, the C18 cartridge was washed with water (30 mL), and the ganglioside product was eluted using a solution of 60% acetonitrile in water. The fractions contained pure target compounds were collected, concentrated, and lyophilized. The obtained 9N₃-GD3 was dissolved in sodium bicarbonate saturated solution in water (3 mL), thioacetic acid (10 equiv.) was added, and the reaction was stirred at 70° C. for 20 hours. An additional amount of thioacetic acid (5 equiv.) was added every 10 hours. After three days, HRMS analysis indicated the completion of the reaction. The solvent was concentrated and purified via a preconditioned Discovery DSC-18 SPE cartridge (bed wt. 10 g) to produce the desired 9NAc-GD3 (1) (15 mg, 86%).

### II. Chemoenzymatic synthesis of 9NAc-GD3βSph (7), 9NAc-GD2βSph (8), 9NAc-GD1bβSph (9), and 9NAc-GT1bβSph (10) using ManNAc6NAc as a Neu5Ac9NAc precursor

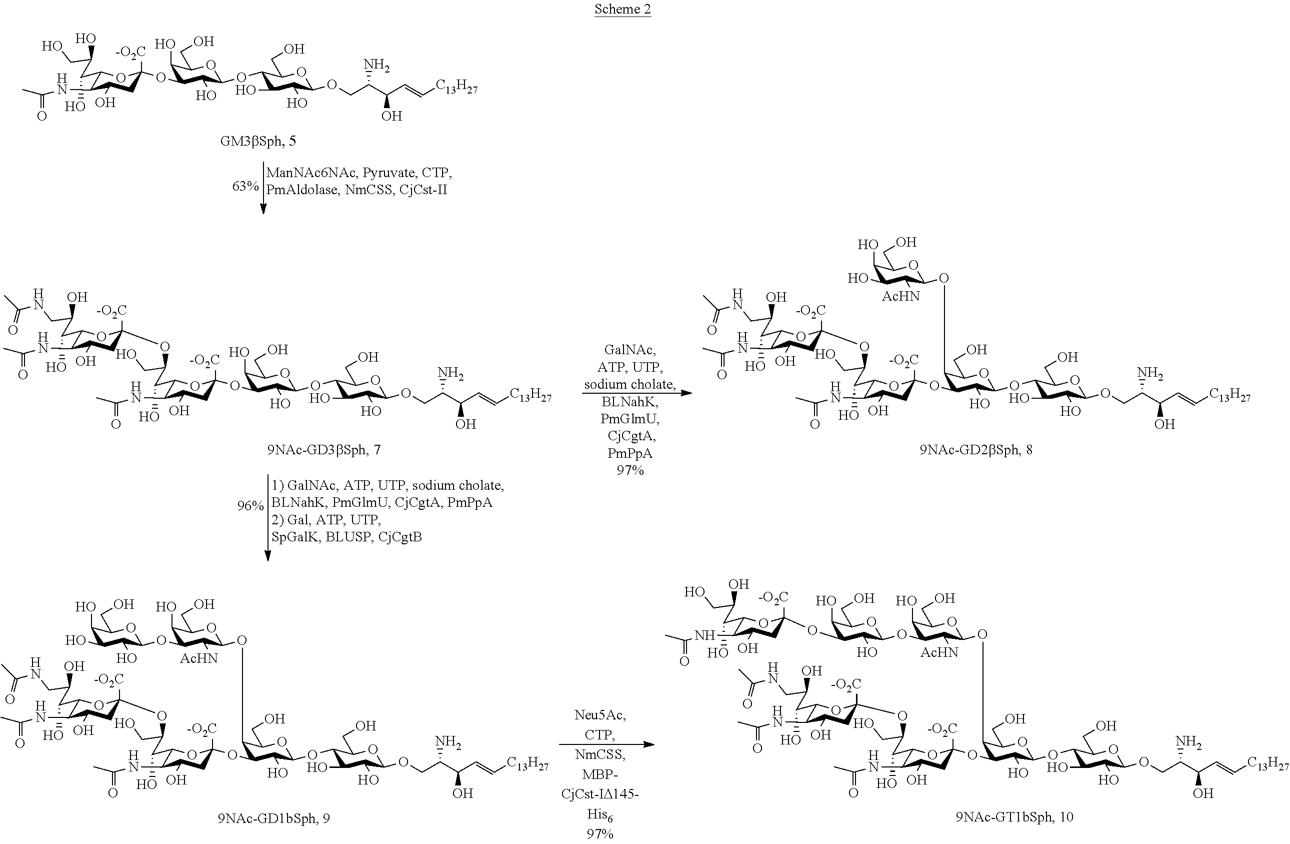

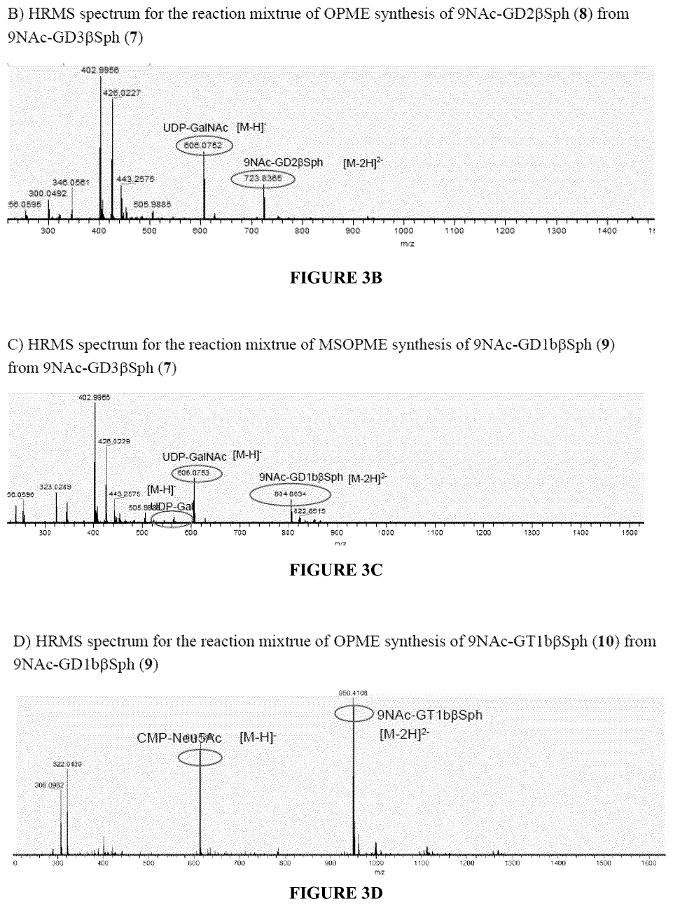

Synthesis of 9NAc-GD3βSph (7) using 6-acetamido-6-deoxy-N-acetylmannosamine (ManNAc6NAc) as a 9-acetamido-9-deoxy-N-acetylneuraminic acid (Neu5Ac9NAc) precursor: Due to the relatively low efficiency in converting the azido group in 9N₃-GD3 to an acetamido group in 9NAc-GD3, an alternative strategy was also explored using ManNAc6NAc as a precursor for enzymatic synthesis of 9NAc-GD3βSph (7) directly from GM3βSph (5). As shown in Scheme 2, 9NAc-GD3βSph (7) was synthesized from GM3βSph (5) as the acceptor substrate and ManNAc6NAc as the donor precursor using the OPME sialylation system containing PmAldolase, NmCSS, and CjCst-II (FIG. 3A). Similar to the case for synthesizing 9N₃-GD3βSph (6), no addition sialylation of the desired product 9NAc-GD3βSph (7) was observed. The sialylated product was readily purified by passing the concentration reaction mixture through a C18 cartridge and eluting with a mixed solvent gradient of CH₃CN in water. 9NAc-GD3βSph (7) (130 mg, 63%) was eluted first with 40% acetonitrile in water (v/v) and the unreacted GM3βSph (5) was eluted with 60% acetonitrile in water (v/v).

OPME synthesis of 9NAc-GD3βSph (7): (5,9-Diacetamido-3,5,9-trideoxy-D-glycero-α-D-gal acto-2-nonulopyranosylonic acid)-(2→8)-(5-acetamido-3,5-dideoxy-D-glycero-α-D-galacto-2-nonulopyranosylonic acid)-(2→3)-β-D-galactopyranosyl-(1→4)-β-D-glucopyranosyl-(1→1)-(2S, 3R, 4E)-2-amino-4-octadecene-1,3-diol: A reaction mixture in Tris-HCl buffer (100 mM, pH 8.5) in a total volume of 16 mL containing GM3βSph (5) (150 mg, 0.16 mmol), ManNAc6NAc (84 mg, 0.32 mmol), sodium pyruvate (211 mg, 1.92 mmol), CTP (202 mg, 0.38 mmol), MgCl₂ (20 mM), PmAldolase (3.2 mg), NmCSS (1.6 mg), and CjCst-II (4 mg) was incubated in an incubator shaker at 30° C. with agitation at 100 rpm. The product formation was monitored by HRMS. After 20 hours, an additional amount of CjCstII (4 mg) was added. After three days, the reaction mixture was incubated in a boiling water bath for 5 minutes and then centrifuged to remove precipitates. The supernatant was concentrated and the residue was purified via a preconditioned Discovery DSC-18 SPE cartridge (bed wt. 10 g). After the sample was loaded, the C18 cartridge was washed with water (30 mL), and 9NAc-GD3βSph (7) was eluted with 40% acetonitrile in water (v/v). Unreacted GM3βSph (5) was eluted with 60% acetonitrile in water (v/v). The fractions containing the pure product were collected, concentrated, and lyophilized to produce the desired pure 9NAc-GD3βSph (7) (130 mg, 63% yield). ¹H NMR (800 MHz, CD₃OD) δ 5.81 (dt, J=14.4, 6.4 Hz, 1H), 5.50 (dd, J=15.2, 6.4 Hz, 1H), 4.50 (d, J=8.0 Hz, 1H), 4.35 (d, J=8.0 Hz, 1H), 4.24-3.12 (m, 29H), 2.93 (dd, J=12.8, 4.8 Hz, 1H), 2.73 (dd, J=12.8, 4.8 Hz, 1H), 2.10 (q, J=7.2 Hz, 2H), 2.04 (s, 3H), 2.02 (s, 3H), 1.99 (s, 3H), 1.73-1.70 (m, 2H), 1.47-1.15 (m, 24H), 0.90 (t, J=7.2 Hz, 3H). ¹³C NMR (200 MHz, CD₃OD) δ 173.98, 173.63, 173.29, 173.15, 172.27, 134.64, 128.34, 103.44, 102.59, 101.06, 100.09, 79.22, 77.01, 75.45, 75.09, 74.64, 74.10, 73.21, 73.03, 71.51, 70.58, 69.68, 69.42, 68.90, 68.25, 68.08, 67.28, 61.99, 61.41, 60.39, 55.07, 52.80, 52.50, 42.69, 41.40, 40.90, 32.04, 31.68, 29.41, 29.37, 29.33, 29.26, 29.08, 29.07, 29.02, 28.93, 22.34, 21.67, 21.32, 21.29, 21.27, 13.08, 13.07. HRMS (ESI-Orbitrap) m/z: [M−H]⁻ calculated for C₅₄H₉₃N₄O₂₈ 1245.5982; found 1245.5957.

Synthesis of 9NAc-GD2βSph (8) from 9NAc-GD3βSph (7) using a one-pot multienzyme (OPME) GaINAc activation and transfer system: With 9NAc-GD3βSph (7) in hand, the synthesis of 9NAc-GD2βSph (8) was accomplished using an OPME Q1-4-GalNAc activation and glycosylation reaction containing Bifidobacterium longum strain ATCC55813 N-acetylhexosamine-1-kinase (BLNahK), Pasteurella multocida N-acetylglucosamine uridylyltransferase (PmGlmU), Pasteurella multocida inorganic pyrophosphatase (PmPpA), and MBP-A15CjCgtA-His6 which was a recombinant Campylobacterjejuni β-4-N-acetylgalactosaminyltransferase designed with improved E. coli expression and increased stability. As shown in Scheme 2, incubating 9NAc-GD3Sph (7), N-acetylgalactosamine (GalNAc), adenosine 5′-triphosphate (ATP), and uridine 5′-triphosphate (UTP) in the reaction mixture containing an anionic detergent sodium cholate at 30° C. produced 9NAc-GD2βSph (8) smoothly (FIG. 3B). Sodium cholate was shown to increase the accessibility of ganglioside substrates by different enzymes, most likely by dispersing gangliosides molecules to decrease steric hindrance. Indeed, similar to what we observed previously, the presence of sodium cholate greatly improved the enzyme properties and the OPME reaction was completed in 16 hours. Pure 9NAc-GD2βSph (8) (56 mg, 97% yield) was readily obtained by removing sodium cholate using a silica gel column chromatography followed by passing the concentrated product-containing fractions through a C18 cartridge and eluting with 30% CH₃CN in water.

OPME synthesis of 9NAc-GD2,8Sph (8): 2-Acetamido-2-deoxy-β-D-galactopyranosyl-(1→4)-(5,9-diacetamido-3,5,9-trideoxy-D-glycero-α-D-galacto-2-nonulopyranosylonic acid)-(2→8)-(5-acetamido-3,5-dideoxy-D-glycero-α-D-galacto-2-nonulopyranosylonic acid)-(2→3)-3-D-galactopyranosyl-(1→4)-β-D-glucopyranosyl-(1→1)-(2S, 3R, 4E)-2-amino-4-octadecene-1,3-diol: 9NAc-GD3βSph (7) (50 mg, 0.039 mmol), GalNAc (13 mg, 0.059 mmol), ATP (36 mg, 0.066 mmol), and UTP (35 mg, 0.066 mmol) were incubated in 5 mL of Tris-HCl buffer (100 mM, pH 7.5) containing BLNahK (1.2 mg), PmGlmU (1.3 mg), MBP-A15CjCgtA-His6 (2 mg), PmPpA (1.0 mg), MgCl₂ (20 mM), and sodium cholate (10 mM). The reaction was carried out by incubating the solution in an incubator shaker at 30° C. for 16 hours with agitation at 100 rpm. The product formation was monitored by HIRMS. After the reaction was completed, the reaction mixture was incubated in a boiling water bath for 5 minutes and then centrifuged to remove precipitates. The supernatant was concentrated and the residue was purified by a silica gel column chromatography. A mixed solvent chloroform:methanol=5:2 (by volume) was used to remove sodium cholate and chloroform:methanol:water=5:4:1 (by volume) was used to elute the product. The fractions containing the product were collected and concentrated. The residue was dissolved in double-distilled water (ddH₂O) (1 mL) and loaded to a preconditioned Discovery DSC-18 SPE cartridge (bed wt. 10 g). After washing with water (30 mL), a mixed solvent of acetonitrile in water (30%) was used as an eluent to obtain pure 9NAc-GD2βSph (8) (56 mg, 97%) as a white powder. ¹H NMR (600 MHz, CD₃OD) δ 5.88 (dt, J=14.4, 6.4 Hz, 1H), 5.50 (dd, J=15.2, 6.4 Hz, 1H), 4.47 (d, J=8.0 Hz, 1H), 4.36 (d, J=8.0 Hz, 2H), 4.27-3.25 (m, 35H), 2.86 (dd, J=12.8, 4.8 Hz, 1H), 2.75 (dd, J=12.8, 4.8 Hz, 1H), 2.10 (q, J=7.2 Hz, 2H), 2.05 (s, 3H), 2.03 (s, 3H), 2.02 (s, 3H), 1.99 (s, 3H), 1.80-1.74 (m, 2H), 1.51-1.20 (m, 24H), 0.90 (t, J=7.2 Hz, 3H). ¹³C NMR (150 MHz, CD₃OD) δ 173.97, 173.50, 173.43, 173.22, 172.86, 172.26, 135.13, 126.94, 103.57, 102.97, 102.43, 101.06, 100.52, 79.05, 77.07, 76.68, 75.20, 74.91, 74.60, 74.39, 74.03, 73.17, 73.05, 72.63, 70.66, 69.55, 69.47, 68.56, 68.35, 68.22, 65.78, 61.96, 61.37, 61.23, 60.38, 60.08, 59.76, 55.39, 53.23, 52.87, 52.62, 42.76, 40.78, 40.06, 31.98, 31.66, 29.38, 29.34, 29.23, 29.05, 29.02, 28.81, 22.32, 22.25, 21.75, 21.30, 21.29, 13.04. HIRMS (ESI-Orbitrap) m/z: [M−2H]²⁻ calculated for C₆₂H₁₀₇N₅O₃₃ 723.8351; found 723.8365.

Synthesis of 9NAc-GD1bβSph (9) from 9NAc-GD3βSph (7) using a multistep one-pot multienzyme (MSOPME) strategy: From 9NAc-GD3βSph (7), a multistep one-pot multienzume (MSOPME) strategy was used to directly access 9NAc-GD1bβSph (9). In the first step, 9NAc-GD2βSph (8) was formed from 9NAc-GD3βSph (7) as described above. When the reaction went to completion, the reaction mixture was used directly in the second step without purification to produce 9NAc-GD1bβSph (9) using an OPME (β-3-galactosylation system by adding galactose (Gal), ATP, UTP, and four enzymes including Streptococcus pneumoniae TIGR4 galactokinase (SpGalK), Bifidobacterium longum UDP-sugar pyrophosphorylase (BLUSP), PmPpA, and MBP-CjCgtBΔ30-His6 which was a recombinant Campylobacter jejuni pβ-3-galactosyltransferase designed with improved E. coli expression and increased stability. The second OPME glycosylation was completed at 30° C. in 20 h (FIG. 3C). The desired pure 9NAc-GD1bβSph (9) (123 mg) was obtained in 96% yield after removing sodium cholate using a silica gel column chromatography followed by purification using a C18 cartridge.

MSOPME synthesis of 9NAc-GD1bβSph (9): β-D-Galactopyranosyl-(1→3)-(2-acetamido-2-deoxy-β-D-galactopyranosyl)-(→4)-(5,9-diacetamido-3,5,9-trideoxy-D-glycero-α-D-galacto-2-nonulopyranosylonic acid)-(2→8)-(5-acetamido-3,5-dideoxy-D-glycero-α-D-galacto-2-nonulopyranosylonic acid)-(2→3)-β-D-galactopyranosyl-(1→4)-β-D-glucopyranosyl-(1→1)-(2S, 3R, 4E)-2-amino-4-octadecene-1,3-diol: A reaction mixture in Tris-HCl buffer (100 mM, pH 7.5) in a total volume of 8 mL containing 9NAc-GD3βSph (7) (100 mg, 0.077 mM), GalNAc (26 mg, 0.116 mmol), ATP (156 mg, 0.132 mmol), UTP (70 mg, 0.132 mmol), BLNahK (2.0 mg), PmGlmU (2.0 mg), MBP-A15CjCgtA-His6 (3.0 mg), PmPpA (1.5 mg), MgCl₂ (20 mM), and sodium cholate (10 mM) was incubated at 30° C. with agitation at 100 rpm. The formation of the product 9NAc-GD2βSph (8) was monitored by HRMS and 9NAc-GD3βSph (7) was completely consumed after 16 hours. In the same reaction container without workup or purification, galactose (21 mg, 0.116 mmol), ATP (73 mg, 0.132 mmol), and UTP (70 mg, 0.132 mmol), SpGalK (1.5 mg), BLUSP (2.0 mg), MBP-CjCgtBΔ30-His6 (3.0 mg), and PmPpA (1.0 mg) were added. The reaction mixture with a total volume of 11 mL was incubated at 30° C. for 20 h with agitation at 100 rpm. The product formation was monitored by HRMS.

After the reaction was completed, the reaction mixture was incubated in a boiling water bath for 5 min and then centrifuged to remove precipitates. The supernatant was concentrated, and the residue obtained was purified by a silica gel column chromatography. A mixed solvent chloroform:methanol=5:2 (by volume) was used to remove sodium cholate and chloroform:methanol:water=5:4:1 (by volume) was used to elute the product. The fractions containing the product were collected and concentrated. The residue was dissolved in ddH₂O (1 mL) and loaded to a preconditioned Discovery DSC-18 SPE cartridge (bed wt. 10 g). After washing with water (30 mL), a mixed solvent of acetonitrile in water (30%) was used as an eluent to obtain pure 9NAc-GD1b βSph (9) (123 mg, 96%) as a white powder. ¹H NMR (600 MHz, CD₃OD) δ 5.88 (dt, J=14.4, 6.4 Hz, 1H), 5.50 (dd, J=15.2, 6.4 Hz, 1H), 4.46-4.34 (m, 4H), 4.27-3.23 (m, 41H), 2.86 (dd, J=12.8, 4.8 Hz, 1H), 2.71 (dd, J=12.8, 4.8 Hz, 1H), 2.11 (q, J=7.2 Hz, 2H), 2.03 (s, 3H), 2.01 (s, 6H), 1.99 (s, 3H), 1.87-1.71 (m, 2H), 1.49-1.22 (m, 24H), 0.90 (t, J=7.2 Hz, 3H). ¹³C NMR (150 MHz, CD₃OD) δ 174.00, 173.47, 173.08, 172.29, 135.16, 126.94, 105.09, 103.66, 102.98, 102.38, 100.92, 81.00, 79.10, 76.80, 75.19, 74.74, 74.61, 74.39, 73.98, 73.15, 71.13, 70.67, 69.48, 68.92, 68.31, 68.20, 65.76, 61.96, 61.26, 61.14, 60.35, 60.09, 59.74, 55.35, 52.91, 52.66, 51.56, 42.74, 40.47, 39.76, 32.00, 31.95, 31.67, 29.39, 29.36, 29.30, 29.25, 29.19, 29.07, 29.03, 28.87, 28.81, 22.44, 22.33, 21.76, 21.32, 13.06. HRMS (ESI-Orbitrap) m/z: [M−2H]²⁻ calculated for C₆₈H₁₁₇N₅O₃₈ 804.8616; found 804.8634. Synthesis of 9NAc-GTJbβSph (10) from 9NAc-GD1bβSph (9) using an OPME sialylation system: To synthesize 9NAc-GT1bβSph (10) from 9NAc-GD1bβSph (9), a suitable α2-3-sialyltransferase was needed. Pasteurella multocida α2-3-sialyltransferases PmST1 and PmST3 had no or very low activity on sialylating the terminal Gal in 9NAc-GD1bβSph (9). A recombinant human ST3GAL II (MBP-Δ27hST3GAL II-His6) was previously cloned which was successfully expressed in E. coli, purified, and used in the synthesis of GT1b glycan from GD1b glycan. Its low expression level and less efficiency in using glycosylsphingosines as acceptors compared to the corresponding glycans, however, limited its application in the synthesis of 9NAc-GT1bβSph (10). It was found that MBP-CjCst-IΔ145-His6, a recombinant Campylobacter jejuni α2-3-sialyltransferase used in sialylating glycoprotein N-glycans, showed very good activity towards sialylating GD1b sphingosine and glycan. Compared to the recombinant MBP-Δ27hST3GAL II-His6 expressed in E. coli which has an expression level of less than 1 mg (˜9 mU) per liter LB culture, MBP-CjCst-IΔ145-His6 has a much higher expression level (60 mg purified enzyme from per liter LB culture). Preparative-scale synthesis of 9NAc-GT1bβSph (10) was achieved efficiently from 9NAc-GD1bβSph (9) and Neu5Ac in the presence of CTP using an OPME sialylation reaction containing NmCSS and MBP-CjCst-IΔ145-His6. The reaction was completed in 15 hours (FIG. 3D) and pure 9NAc-GT1bβSph (10) (58 mg, 97%) was obtained after purification via a C18 cartridge by eluting with 20% of CH₃CN in water.

OPME synthesis of 9NAc-GTJbβSph (10): (5-acetamido-3, 5-dideoxy-D-glycero-α-D-galacto-2-nonulopyranosylonic acid)-(2→3)-β-D-Galactopyranosyl-(1→3)-(2-acetamido-2-deoxy-β-D-galactopyranosyl)-(1→4)-(5,9-diacetamido-3,5,9-trideoxy-D-glycero-α-D-galacto-2-nonulopyranosylonic acid)-(2→8)-(5-acetamido-3,5-dideoxy-D-gly cero-α-D-galacto-2-nonulopyranosylonic acid)-(2→3)-β-D-galactopyranosyl-(1→4)-β-D-glucopyranosyl-(1→1)-(2S, 3R, 4E)-2-amino-4-octadecene-1,3-diol. 9NAc-GD1bβSph (9) (50 mg, 0.030 mmol), Neu5Ac (14 mg, 0.045 mmol), and CTP (27 mg, 0.051 mmol) were incubated at 37° C. in a Tris-HCl buffer (6 mL, 100 mM, pH 8.5) containing MgCl₂ (20 mM), NmCSS (0.8 mg), and MBP-CjCst-IΔ145-His6 (2 mg). The reaction mixture was incubated in an incubator shaker at 30° C. for 15 hours with agitation at 100 rpm. The product formation was monitored by HRMS. Upon completion, the reaction mixture was incubated in a boiling water bath for 5 minutes and then centrifuged to remove precipitates. The supernatant was concentrated and the residue was purified via a preconditioned Discovery DSC-18 SPE cartridge (bed wt. 10 g). After the sample was loaded, the C18 cartridge was washed with water (30 mL), a mixed solvent of acetonitrile in water (20%) was then used as an eluent to obtain pure 9NAc-GT1bβSph (10) (58 mg, 98%) as a white powder. ¹H NMR (600 MHz, CD₃OD) δ 5.86 (dt, J=14.4, 6.4 Hz, 1H), 5.50 (dd, J=15.2, 6.4 Hz, 1H), 4.51 (d, J=8.0 Hz, 1H), 4.46 (d, J=8.0 Hz, 1H), 4.35 (d, J=8.0 Hz, 1H), 4.28 (s, 1H), 4.24-3.21 (m, 48H), 2.94-2.66 (m, 3H), 2.11 (q, J=7.2 Hz, 2H), 2.04 (s, 3H), 2.02 (s, 3H), 1.99 (s, 3H), 1.79-1.71 (m, 3H), 1.49-1.22 (m, 24H), 0.90 (t, J=7.2 Hz, 3H). ¹³C NMR (150 MHz, CD₃OD) δ 174.05, 173.48, 173.42, 173.02, 172.26, 134.90, 127.41, 104.58, 103.51, 102.93, 102.52, 101.02, 100.41, 99.68, 76.78, 76.13, 75.21, 74.60, 74.33, 73.83, 73.49, 73.17, 71.51, 70.54, 70.10, 69.65, 69.31, 68.52, 68.30, 68.07, 67.96, 67.54, 66.57, 62.95, 62.06, 61.30, 60.63, 60.34, 55.22, 52.93, 52.55, 51.36, 42.62, 40.89, 32.04, 31.68, 29.41, 29.37, 29.33, 29.27, 29.08, 29.06, 28.88, 22.59, 22.34, 21.79, 21.41, 21.36, 13.08. HRMS (ESI-Orbitrap) m/z: [M-2H]²⁻ calculated for C₇₉H₁₃₄N₆O₄₆ 950.4093; found 950.4108.

### III. Acylation for converting glycosylsphingosines to glycosphingolipids

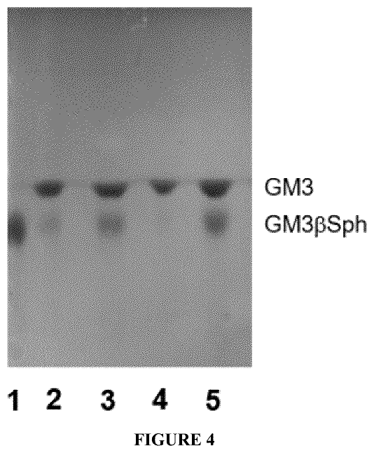

With 9-N-acetylated GD3βSph, GD2bβSph, GD1bβSph, and GT1bβSph (7-10) in hand, the final step for the formation of target 9-N-acetylated GD3, GD2b, GD1b, and GT1b (1-4) was acylation. The acylation conditions using THF/saturated aqueous NaHCO₃led to high yields (97-100%). Nevertheless, a large amount of the salt was introduced to the reaction mixture which formed a two-phase mixture. The reaction conditions had room for improvement, especially for large-scale reactions. Using GM3βSph (5) as a model substrate, several acylation conditions were compared, including THE/saturated NaHCO₃ (1:1 by volume), THF/50 mM carbonate buffer (pH 9.2) (1:1 by volume), THF/100 mM CAPS buffer (pH 9.5) (1:1 by volume), and THE/1% Na₂CO₃ (1:1 by volume). The reaction mixtures were analyzed by thin-layer chromatography (FIG. 4). Among these conditions, THE/1% Na₂CO₃ (1:1 by volume) was identified as the optimal condition, with which the acylation rate was the fastest and the reaction for the formation of GM3 was completed in one hour. Furthermore, the reaction mixture was a homogeneous solution without any undissolved residues. Using the improved acylation reaction conditions (stearoyl chloride in THF/1% Na₂CO₃), the production of target gangliosides 9NAc-GD3 (1), 9NAc-GD2 (2), 9NAc-GD1b (3), and 9NAc-GT1b (4) (d18:1-18:0) was achieved by installing a stearoyl chain to the amino group in the corresponding glycosylsphingosines. The acylation reaction progress was monitored by HRMS and additional amounts of stearoyl chloride were added during the acylation reaction when needed. The reactions for the production of more complex glycosphingolipids such as 9NAc-GD1b (3) and 9NAc-GT1b (4) took longer time (3-4 h) to complete compared to less complex glycosphingolipids such as 9NAc-GD3 (1), 9NAc-GD2 (2) (reactions were completed in 2 h). The desired products were purified using C18 cartridges.

Optimizing the acylation reaction conditions for the formation of gangliosides from sphingosines using GM3βSph as a model substrate: Acylation reaction assays were performed at room temperature for 1 h in a 3 mL of solution containing GM3Sph (2 mg) and stearoyl chloride (1.5 equiv.). Solution used were: THE/sat NaHCO₃; THE/50 mM carbonate buffer (pH 9.2); THE/100 mM CAPS buffer (pH9.5); and THE/1% Na₂CO₃. The reactions were analyzed by thin-layer chromatography. Developing solvent used was i-PrOH:H₂O:NH₄OH=10:2:1.

General procedures for converting glycosylsphingosines (7-10) to gangliosides (1-4): To a solution of a glycosylsphingosine selected from 7-10 (15-20 mg) in THF/i % Na₂CO₃ (3 mL,1:1), stearoyl chloride (1.5 eq) in 0.1 mL of THF was added. The resulting mixture was stirred vigorously at room temperature for 1.5 hours. The solution was then concentrated and dissolved in double distilled water (ddH₂O; 2 mL). The product formation was monitored by HRMS. An additional amount of stearoyl chloride (0.5 eq) in THE was added. The reaction was completed in 2 hours for 9NAc-GD3βSph (7) and 9NAc-GD2βSph (8), and in 4 hours for 9NAc-GD1bβSph (9) and 9NAc-GT1bβSph (10). The product was purified via a preconditioned Discovery DSC-18 SPE cartridge (bed wt. 10 g). After the sample was loaded, the C18 cartridge was washed with water (30 mL), and the ganglioside product was eluted using a solution of 40-80% acetonitrile in water. The product was further purified by a silica gel column chromatography using chloroform:methanol=5:2 (by volume) then chloroform:methanol:water=5:4:1 (by volume) as an eluant.

9NAc-GD3 (1): The product 9NAc-GD3 (1) was eluted from the C18 cartridge using 50% CH₃CN in water. The pure product (24 mg, 99% yield) was a white powder. ¹H NMR (800 MHz, CD₃OD) δ 5.68 (dt, J=15.2, 6.4 Hz, 1H), 5.44 (dd, J=15.2, 8.0 Hz, 1H), 4.49 (d, J=8.0 Hz, 1H), 4.31 (d, J=8.0 Hz, 1H), 4.24-3.28 (m, 23H), 3.21 (dd, J=12.6, 8.0 Hz, 1H), 2.92 (dd, J=12.0, 4.0 Hz, 1H), 2.73 (dd, J=12.0, 4.0 Hz, 1H), 2.16 (t, J=7.2 Hz, 2H), 2.02 (s, 3H, CH₃), 2.01 (s, 3H, CH₃), 1.99 (s, 3H, CH₃), 1.72-1.67 (m, 2H), 1.62-1.55 (m, 2H), 1.41-1.21 (m, 50H), 0.90 (t, J=7.2 Hz, 6H). ¹³C NMR (200 MHz, CD₃OD) δ 174.53, 173.95, 173.61, 173.22, 172.30, 133.65, 130.00, 103.45, 103.04, 100.81, 100.08, 79.35, 76.99, 75.44, 75.37, 75.00, 74.75, 74.13, 73.44, 72.98, 71.55, 70.66, 69.55, 69.45, 68.54, 68.31, 68.26, 67.20, 62.10, 61.41, 60.33, 53.29, 52.80, 52.49, 42.76, 41.46, 40.84, 37.96, 35.98, 32.08, 31.71, 31.69, 29.49, 29.46, 29.44, 29.43, 29.41, 29.39, 29.38, 29.35, 29.32, 29.28, 29.25, 29.12, 29.09, 29.07, 29.04, 26.45, 25.78, 22.83, 22.36, 22.35, 22.33, 21.60, 21.19, 13.07, 13.06, 13.03. HRMS (ESI-Orbitrap) m/z: [M-2H]²⁻ calculated for C₇₂H₁₂₈N₄O₂₉ 755.4259; found 755.4267.

9NAc-GD2 (2): The product 9NAc-GD2 (2) was eluted from the C18 cartridge using 40% CH₃CN in water. The pure product (22 mg, 97% yield) was a white powder. ¹H NMR (600 MHz, CD₃OD) δ 5.69 (dt, J=14.4, 6.4 Hz, 1H), 5.44 (dd, J=15.2, 6.4 Hz, 1H), 4.43 (d, J=8.0 Hz, 1H), 4.30 (d, J=8.0 Hz, 1H), 4.19-3.25 (m, 36H), 2.85 (dd, J=12.8, 4.8 Hz, 1H), 2.71 (dd, J=12.8, 4.8 Hz, 1H), 2.19-2.04 (m, 4H), 2.04 (s, 3H), 2.03 (s, 3H), 2.01 (s, 3H), 1.99 (s, 3H), 1.83-1.68 (m, 2H), 1.61-1.20 (m, 52H), 0.90 (t, J=7.2 Hz, 6H). ¹³C NMR (150 MHz, CD₃OD) δ 174.52, 173.94, 173.40, 172.34, 133.62, 130.01, 103.64, 103.09, 102.91, 79.45, 78.06, 76.71, 75.06, 74.90, 74.69, 74.34, 73.46, 73.12, 72.60, 71.54, 70.59, 69.47, 68.54, 68.20, 62.14, 61.19, 60.29, 53.30, 53.23, 52.63, 48.17, 42.73, 39.79, 35.98, 32.08, 31.70, 31.68, 29.49, 29.45, 29.43, 29.41, 29.37, 29.31, 29.24, 29.18, 29.11, 29.08, 29.04, 25.78, 22.36, 22.34, 22.17, 21.66, 21.22, 13.06. HRMS (ESI-Orbitrap) m/z: [M−2H]²⁻ calculated for C₈₀H₁₄₁N₅O₃₄ 856.9656; found 856.9665.

9NAc-GD1b (3): The product 9NAc-GD1b (3) was eluted from the C18 cartridge using 40% CH₃CN in water. The pure product (22 mg, 97% yield) was a white powder. ¹H NMR (600 MHz, CD₃OD) δ 5.68 (dt, J=14.4, 6.4 Hz, 1H), 5.44 (dd, J=15.2, 6.4 Hz, 1H), 4.43 (d, J=8.0 Hz, 2H), 4.29 (d, J=8.0 Hz, 1H), 4.19-3.26 (m, 42H), 2.85 (dd, J=12.8, 4.8 Hz, 1H), 2.67 (dd, J=12.8, 4.8 Hz, 1H), 2.19-2.14 (m, 4H), 2.03 (s, 3H), 2.01 (s, 6H), 2.00 (s, 3H), 1.84-1.72 (m, 2H), 1.62-1.23 (m, 52H), 0.90 (t, J=7.2 Hz, 6H). ¹³C NMR (150 MHz, CD₃OD) δ 174.52, 173.95, 173.47, 172.31, 133.63, 130.00, 105.02, 103.73, 103.08, 102.93, 79.48, 78.06, 75.26, 75.06, 74.68, 74.59, 74.31, 73.82, 73.46, 73.17, 71.54, 71.13, 69.47, 68.97, 68.54, 68.20, 62.14, 61.25, 60.26, 53.30, 52.67, 51.56, 48.17, 42.69, 35.98, 32.08, 31.73, 31.70, 31.68, 29.48, 29.45, 29.42, 29.41, 29.37, 29.31, 29.24, 29.11, 29.08, 29.03, 25.78, 22.35, 22.34, 21.67, 21.31, 21.24, 13.07, 13.06. HRMS (ESI-Orbitrap) m/z: [M−2H]²⁻ calculated for C₈₆H₁₅₁N₅O₃₉ 937.9920; found 937.9934.

9NAc-GT1b (4): The product 9NAc-GT1b (4) was eluted from the C18 cartridge using 40% CH₃CN in water. The pure product (16 mg, 95% yield) was a white powder. ¹H NMR (600 MHz, CD₃OD) δ 5.69 (dt, J=14.4, 6.4 Hz, 1H), 5.45 (dd, J=15.2, 6.4 Hz, 1H), 4.60-4.28 (m, 4H), 4.20-3.26 (m, 49H), 2.91-2.70 (m, 3H), 2.19-2.02 (m, 4H), 2.02 (s, 9H), 2.01 (s, 3H), 1.99 (s, 3H), 1.85-1.72 (m, 3H), 1.62-1.22 (m, 52H), 0.90 (t, J=7.2 Hz, 6H). ¹³C NMR (150 MHz, CD₃OD) δ 174.56, 174.11, 174.01, 173.43, 172.50, 133.71, 129.97, 104.30, 103.66, 103.09, 99.78, 79.38, 76.18, 75.22, 75.04, 74.65, 74.57, 74.28, 73.44, 73.22, 71.53, 70.32, 69.46, 68.58, 68.00, 63.16, 62.30, 61.56, 61.31, 60.23, 53.28, 52.64, 52.52, 51.19, 48.46, 48.18, 42.56, 40.71, 35.99, 32.08, 31.70, 31.67, 29.48, 29.45, 29.42, 29.40, 29.37, 29.36, 29.30, 29.28, 29.25, 29.10, 29.08, 29.03, 25.79, 22.35, 22.34, 21.65, 21.37, 21.34, 21.26, 13.08. HRMS (ESI-Orbitrap) m/z: [M−2H]²⁻ calculated for C₉₇H₁₆₈N₆O₄₇ 1083.5397; found 1083.5401.

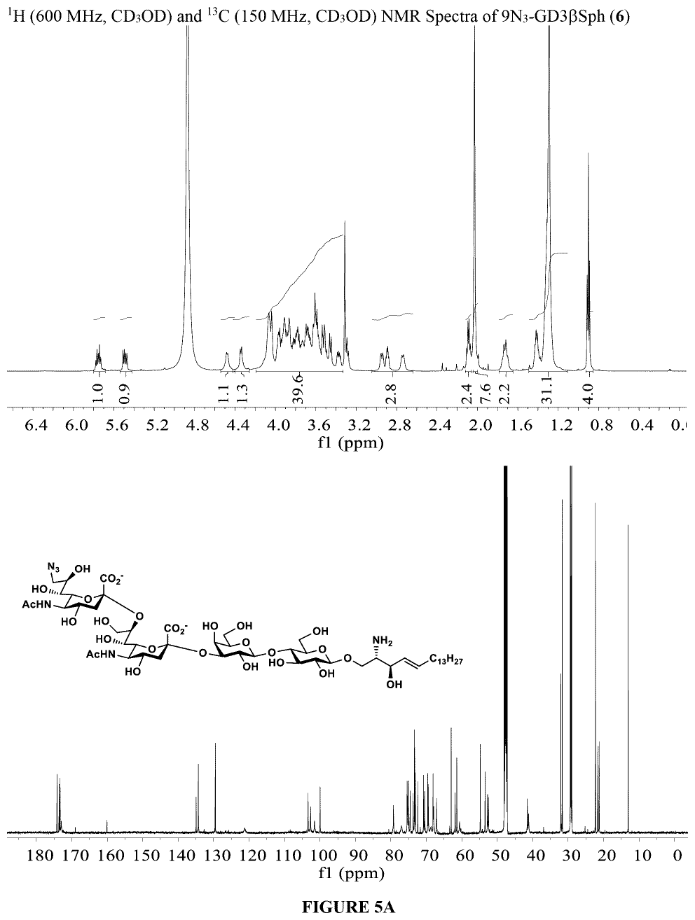

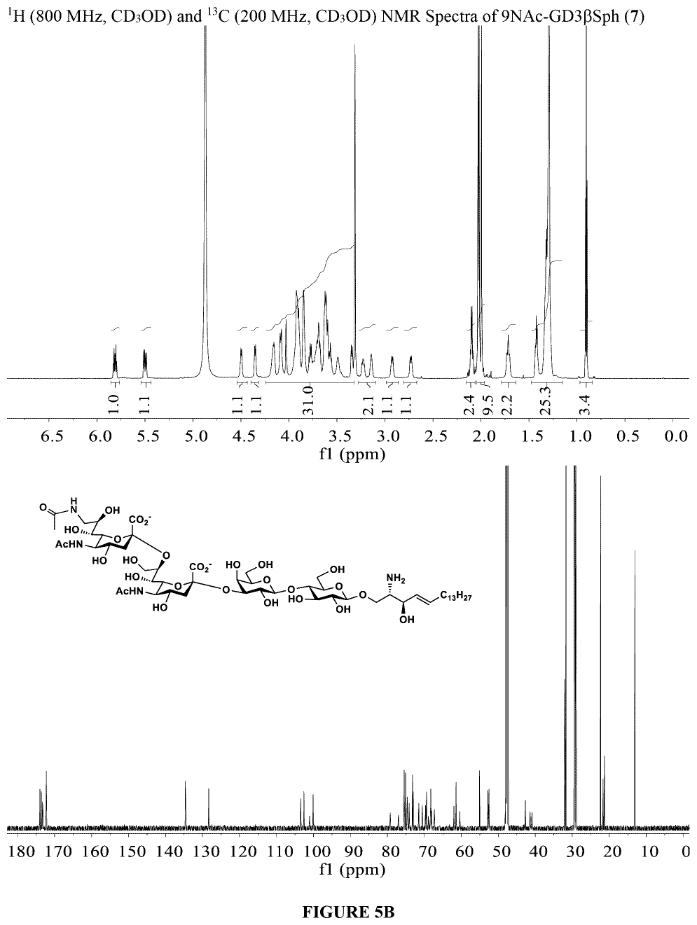

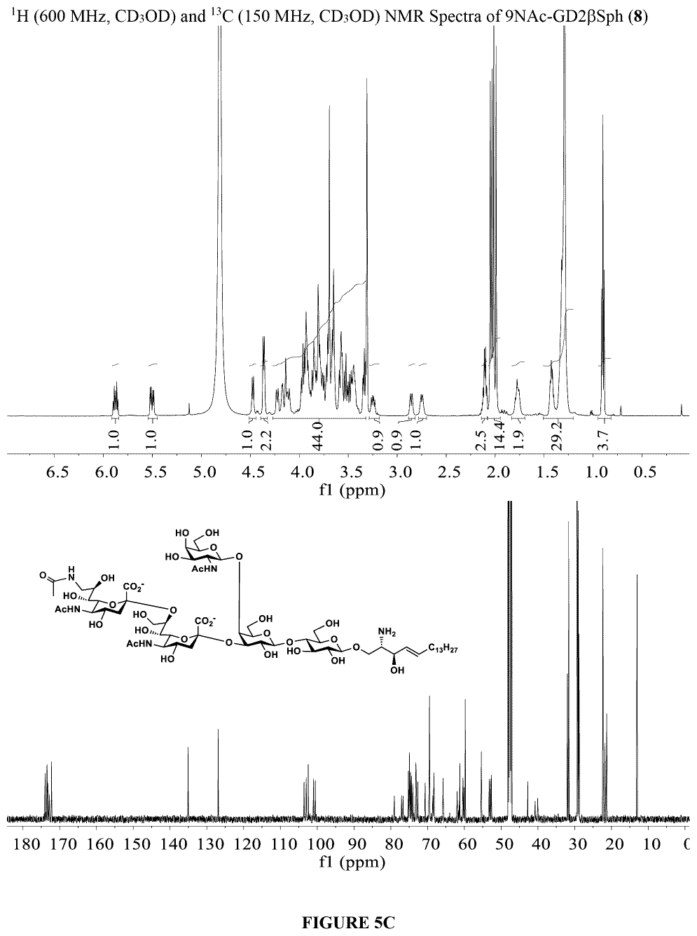

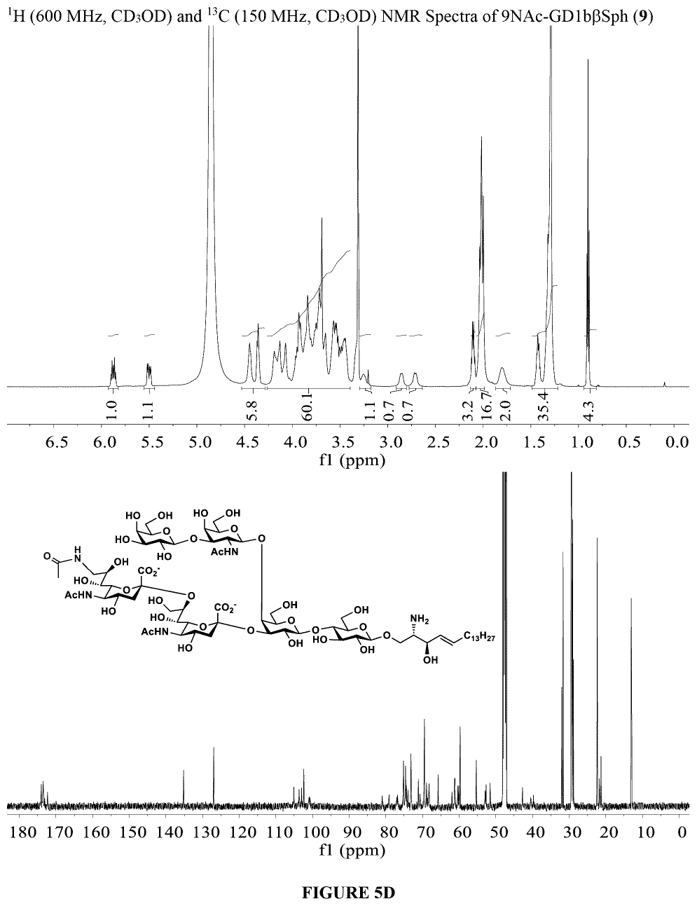

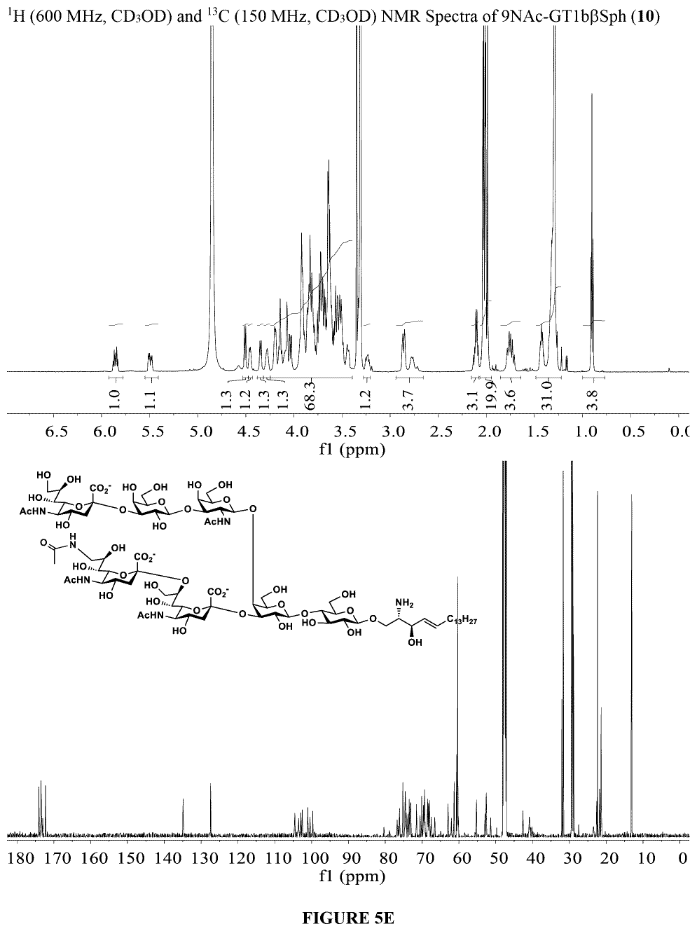

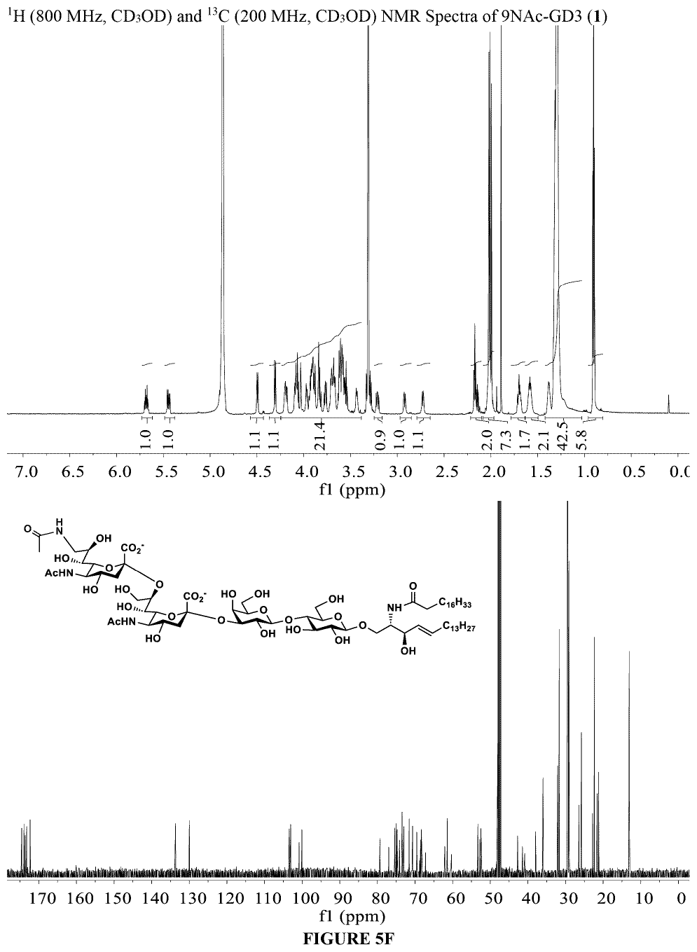

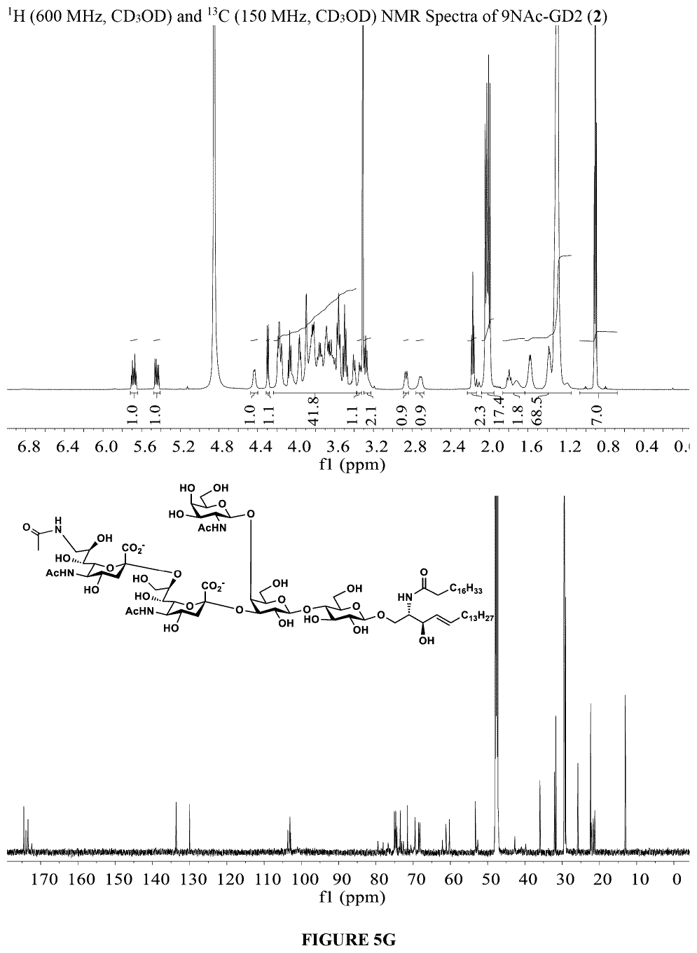

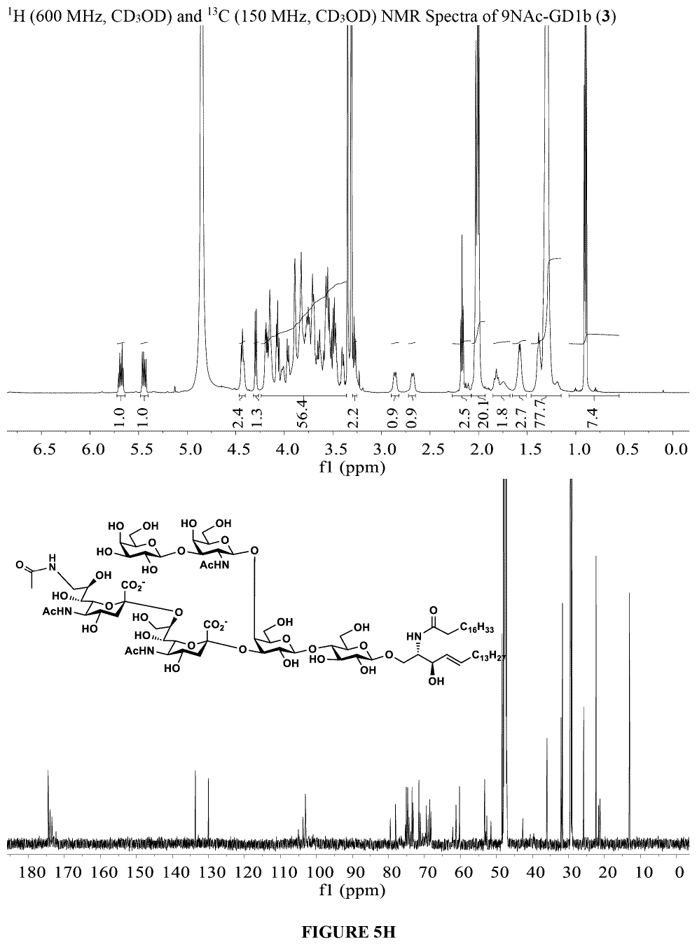

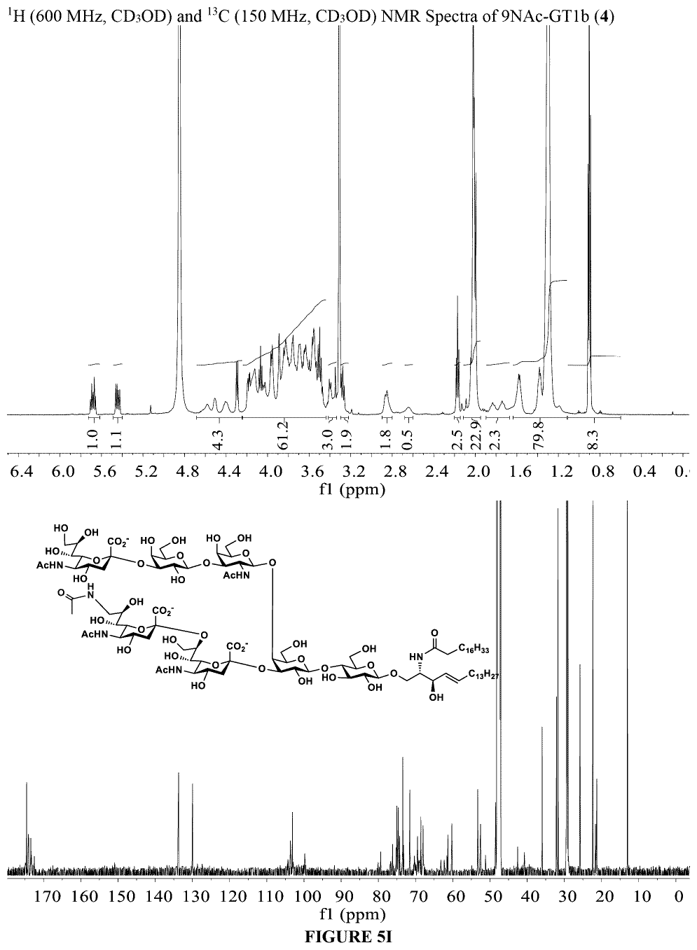

¹H-NMR and ¹³C-NMR spectra for compounds 9N₃-GD3βSph (6); 9NAc-GD3βSph (7); 9NAc-GD2βSph (8); 9NAc-GD1bβSph (9); 9NAc-GT1bβSph (10); 9NAc-GD3 (1); 9NAc-GD2 (2); 9NAc-GD1b (3); and 9NAc-GT1b (4) are provided in FIGS. 5A-5I, respectively.

### Example 2: Substrate Specificity Studies for Sialyltransferases

Donor substrate specificity studies were performed using in situ-generated CMP-sialic acids and derivatives from N₃/NAc-substituted ManNAc (using a one-pot three-enzyme sialylation system) or Neu5Ac derivatives (using a one-pot two-enzyme sialylation system) as donor precursors. The precursors used in the study are shown below:

### Structures of Compounds Used as Precursors for Sialyltransferase Donors

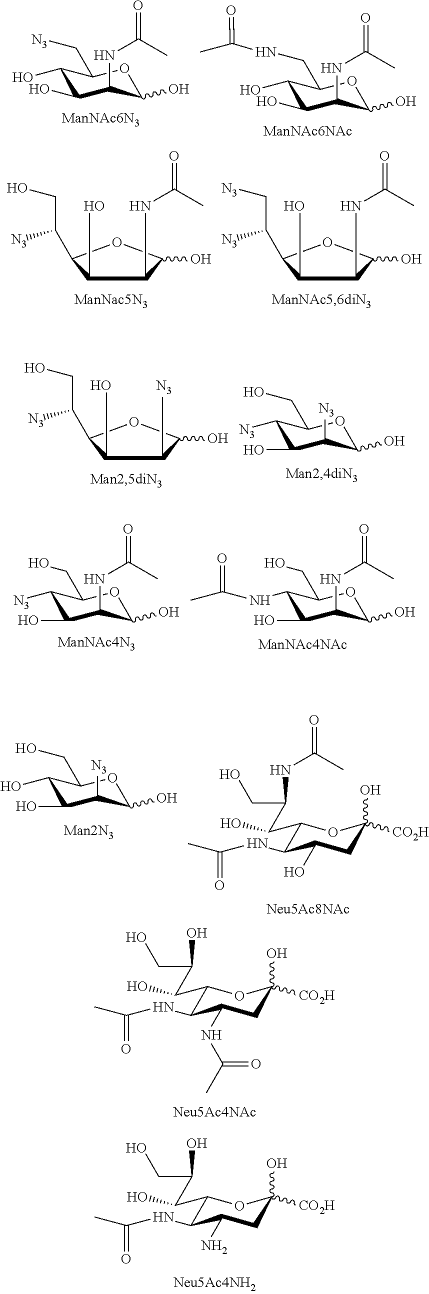

The results for the studies, along with synthetic schemes and the enzymes, are shown below in Table 1 and Table 2.

TABLE 1
Donor substrate specificity studies of PmST1, PmST1_M144D, PmST3, and CjCst-I
using in situ-generated CMP-sialic acids and derivatives from N₃/NAc-substituted ManNAc
(using a one-pot three-enzyme sialylation system) or Neu5Ac derivatives (using a one-pot two-
enzyme sialylation system) as donor precursors. LacßProN₃ or LacßSph was used as the acceptor
substrate.

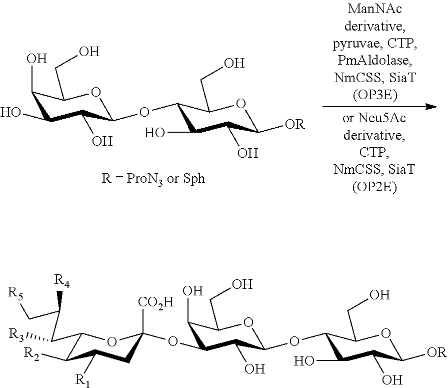

| | LacßProN₃ (SiaT: | | | | | |
|---|---|---|---|---|---|---|
| | ªPmST1 or | | LacßSph | | | |
| | ^{b}pmST1 M144D) | | (SiaT:PmST3) | | LacßSph (SiaT:CjCst-I) | |
| | CMP-Sia | | CMP-Sia | | CMP-Sia | |
| | formation | Sialylation | formation | Sialylation | formation | Sialylation |
| ManNAc6N₃ | Good | ^{b}Over 90% | Good | Over 70% | Good | Over 90% |
| ManNAc6NAc | Good | ^{b}Over 90% | Good | Less than | Good | 60-70% |
| | | | | 10% | | |
| ManNAc5N₃ | Good | ^{b}Over 90% | Good | Less than | Good | Less than |
| | | | | 5% | | 5% |
| ManNAc5,6diN₃ | Good | ^{b}Over 90% | Good | No reaction | Good | No reaction |
| Man2,5diN₃ | Low | bLess than | Low | Less than | Low | Less than |
| | | 5% | | 10% | | 10% |
| Man2,4diN₃ | Good | ^{b}20-30% | Good | 50-60% | Good | 60-70% |
| ManNAc4N₃ | Good | ^{b}Over 80% | Good | Over 70% | Good | Over 80% |
| Man2N₃ | Good | 20-30% | Good | 10-20% | Good | Over 70% |
| Neu5Ac8NAc | Good | ª40-50% | Good | No reaction | Good | Less than |
| | | | | | | 5% |
| Neu5Ac4NAc | Good | ªNo | Good | Less than | Good | Less than |
| | | reaction | | 20% | | 20% |
| Neu5Ac4NH₂ | Good | ª50-60% | Good | 60%-70% | Good | 60%-70% |

For the assay using LacßProN₃, ^{a}wild-type PmST1 was used for Neu5Ac4NH₂, Neu5Ac4NAc, and Neu5Ac8NAc, ^{b}PmST1_M144D was used for other donors.

TABLE 2
Donor substrate specificity studies of CjCst-II using in situ-generated CMP-sialic acids
and derivatives from N₃/NAc-substituted ManNAc (using a one-pot three-enzyme sialylation
system) or Neu5Ac derivatives (using a one-pot two-enzyme sialylation system) as donor
precursors. GM3βBProN₃ or GM3βSph was used as the acceptor substrate.

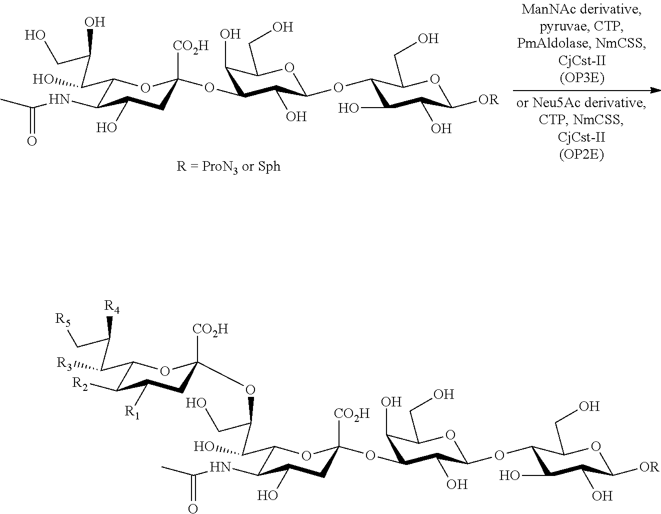

| | GM3βProN3 | GM3ßSph |
|---|---|---|
| ManNAc6N₃ | Over 80% | 65% |
| ManNAc6NAc | Over 80% | 63% |
| ManNAc5N₃ | No reaction | No reaction |
| Man2,4diN₃ | Over 90% | 50 to 60% |
| ManNAc4N₃ | Over 90% | 50 to 60% |
| ManNAc4NAc | No reaction | No reaction |
| Man2N₃ | Over 90% | 70- 80% |
| Neu5Ac8NAc | Not tested | No reaction |
| Neu5Ac4NAc | Less than 5% | No reaction |
| Neu5Ac4NH₂ | 20-30% | Not tested |

### Example 3: Chemical Synthesis of ManNAc4N₃

The synthesis of 2-acetamido-4-azido-2,4-dideoxy-D-mannopyranoside (ManNAc4N₃, 16) from N-acetylmannosamine (ManNAc, 11) was carried out as described below. As shown in Scheme 3, selective tosylation of C6-OH of ManNAc (11) by treating it in pyridine with p-toluene sulfonyl chloride (p-TsCl) at room temperature for 12 hours formed compound 12 with a 63% yield, which was used for the formation of 1,6-anhydride 3 in 77% yield by treating with 1,8-diazabicycloundec-7ene (DBU) at room temperature for 14 h. Dehydration of the C3 and C4 hydroxyl groups by treating anhydride 13 in anhydrous THE with triphenyl phosphine (Ph₃P) in diisopropylazodicarboxylate (DIAD) for 1 hour at 0° C. and then additional 1.5 hours at room temperature formed compound 14 in 73% yield. Epoxide ring opening by SN₂ attacking the C-4 of compound 14 with sodium azide in the presence of Dowex H⁺ resin followed by acetyl protection of the C3-hydroxyl group formed compound 15 in 76% yield over two steps. Acetolysis of compound 15 using acetic anhydride in TMSOTf followed by removal of O-acetyl groups using Dowex H⁺ resin in 25 mM aqueous hydrogen chloride (HCl) produced the desired target ManNAc4N₃ (16) in 51% yield over two steps.

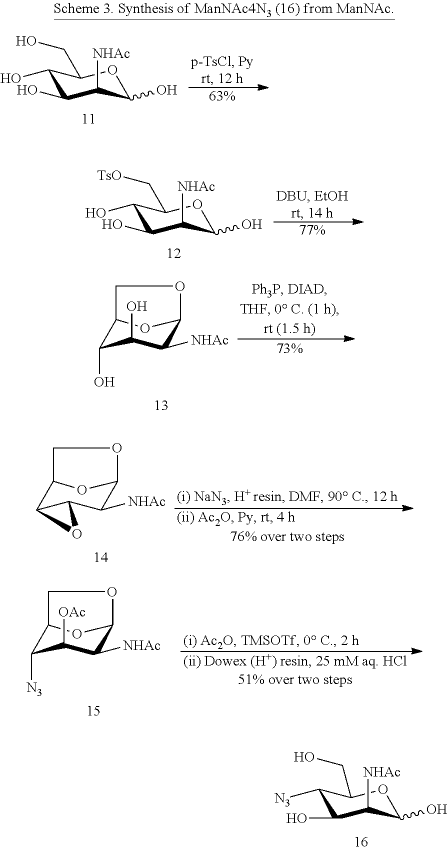

### Synthesis of ManNAc4NAc and Man2,4diN₃

The syntheses of ManNAc4NAc and Man2,4diN₃ were completed as reported in Kooner, A. S., Diaz, S., Yu, H., Santra, A., Varki, A. and Chen, X. (2021), Chemoenzymatic synthesis of sialosides containing 7-N- or 7,9-di-N-acetyl sialic acid as stable O-acetyl analogues for probing sialic acid-binding proteins, J Org Chem 86, 14381-14397, which is incorporated by reference in its entirety.

### Experimental Procedures

### Synthesis of 2-acetamido-4-azido-2,4-dideoxy-D-mannopyranoside (ManNAc4N₃, 16)

To a stirred solution of ManNAc, 11 (8 g, 36.16 mmol) in anhydrous pyridine (50 mL) at 0° C., a solution of p-toluene sulfonyl chloride (11.03 g, 57.85 mmol, 1.6 equiv.) in pyridine (20 mL) was added and reaction mixture was stirred at room temperature (22° C.) for 12 hours. Further, the reaction was quenched by adding MeOH (10 mL) and concentrated using rotavapor. The obtained residue was subjected to column chromatography (EtOAc:MeOH=9:1, by volume) purification to obtain the desired 6-O-tosylated compound 12 (8.55 g, 63%).

The resulting 6-O-tosylated compound 12 (8.5 g, 22.6 mmol) was dissolved in anhydrous ethanol (100 mL), followed by drop wisely addition of 1,8-diazabicycloundec-7ene (DBU) (6.74 mL, 45.2 mmol, 2.0 equiv.) at 0° C. The reaction mixture was stirred at room temperature (22° C.) for 14 hours. After the reaction was completed (as monitored by TLC, Rf=0.3, 10% MeOH/EtOAc), the reaction mixture was concentrated and subjected to column chromatography (EtOAc:MeOH=9:1, by volume) purification to obtain 1,6-anhydro compound 13 (3.35 g, 77%).

To a stirred solution of 1,6-anhydro compound 13 (3.3 g, 16.4 mmol) and Ph₃P (5.16 g, 19.6 mmol, 1.2 equiv.) in anhydrous THE (40 mL), diisopropylazodicarboxylate (DIAD) (3.86 mL, 19.6 mmol, 1.2 equiv.) was added drop-wisely at 0° C. The reaction mixture was stirred at 0° C. for 1 hour and then at room temperature (22° C.) for additional 1.5 hours. After complete consumption of the starting material (monitored by TLC, Rf=0.4, EtOAc). The resulting reaction mixture was concentrated and purified through silica gel column chromatography (EtOAc only) to obtain the epoxide 14 (2.2 g, 73%).

To a stirred solution of compound 14 (2.2 g, 11.8 mmol) in dried DMF (20 mL), NaN₃ (3.08 g, 47.5 mmol) and Dowex H⁺ resin (5 g) were added to the solution. The suspension was stirred at 90° C. for the 12 hours, after the reaction was completed (as monitored by TLC, Rf=0.4, MeOH:EtOAc=0.5:9.5, by volume), the reaction mixture was cooled down to room temperature (22° C.), neutralized with a pinch of NaHCO₃, filtered through celite, and washed with ethyl acetate and methanol. Solvent was evaporated, and the crude residue was dried under high vacuum. The resulting dried residue was further acetylated without column chromatography. The obtained residue was dissolved in pyridine (40 mL) followed by acetic anhydride (6 mL) addition at 0° C., stirred for 4 hours, and reaction progress was monitored by TLC, (Rf=0.5, hexane:EtOAc=2:8, by volume). After the reaction was completed, the reaction mixture was diluted with MeOH (6 mL) and undergo evaporation to obtain the crude residue. The resulting residue was subjected to silica gel column chromatography (hexane:EtOAc=3:7, by volume) purification to obtain compound 15 (2.44 g, 76%). ¹H NMR (400 MHz, CDCl₃) δ 5.83 (d, J=9.6 Hz, 1H), 5.37 (s, 1H), 5.09-5.07 (m, 1H), 4.62-4.61 (m, 1H), 4.49 (ddd, J=9.7, 5.8, 1.9 Hz, 1H), 4.06-4.04 (m, 1H), 3.88-3.84 (m, 1H), 2.18 (s, 3H), 2.03 (s, 3H). ¹³C NMR (100 MHz, CDCl₃) δ 169.6, 169.5, 100.7, 73.8, 69.4, 65.8, 60.5, 46.6, 23.2, 21.0.

To a stirred solution of compound 15 (2.44 g, 9.0 mmol) in acetic anhydride (12 mL), trimethylsilyltrifluoromethanesulfonate (TMSOTf) (1.64 mL, 9.0 mmol) was added dropwise at 0° C. The reaction mixture was stirred at 0° C. for 2 hours. After the reaction was completed (as monitored by TLC), the reaction was quenched by adding saturated NaHCO₃ (50 mL) over 30 minutes. The mixture was extracted with ethyl acetate, washed with saturated NaHCO₃ and brine. The resulting residue was used for the next step without column purification. The crude product and Dowex (H⁺) resin (3 g) in 25 mM HCl (250 mL) were heated at 70° C. for 24 hours. The reaction was monitored by TLC (Rf=0.4, Developing solvent was MeOH:DCM=1.5:8.5, by volume). The mixture was filtered through celite and the celite was washed with water and methanol. The filtrate was collected, and condensed to dryness under reduce pressure. The crude residue was subjected to column chromatography (DCM:MeOH=9:1 to 8.5:1.5, by volume) purification to obtain the pure target product ManNAc4N₃ (16) (1.13 g, 51%). ¹H NMR (400 MHz, D₂O) δ 5.16 (d, J=1.6 Hz, 0.6H), 5.00 (d, J=2.0, 0.4H), 4.48 (dd, J=4.4, 1.7 Hz, 0.4H), 4.35-4.33 (m, 0.6H), 4.20 (dd, J=10.3, 4.6 Hz, 0.4H), 4.01 (dd, J=10.1, 4.3 Hz, 0.5H), 3.89-3.83 (m, 3H), 3.60 (t, J=10.4 Hz, 0.6H), 3.51 (t, J=10.3 Hz, 0.4H), 3.41-3.47 (m, 0.5H), 2.13 (s, 1.3H), 2.09 (s, 1.7H). ¹³C NMR (100 MHz, D₂O) δ 175.7, 174.7, 93.1, 92.9, 74.6, 71.6, 70.30, 68.2, 60.7, 60.6, 59.1, 58.7, 53.77, 52.70, 22.0, 21.8.

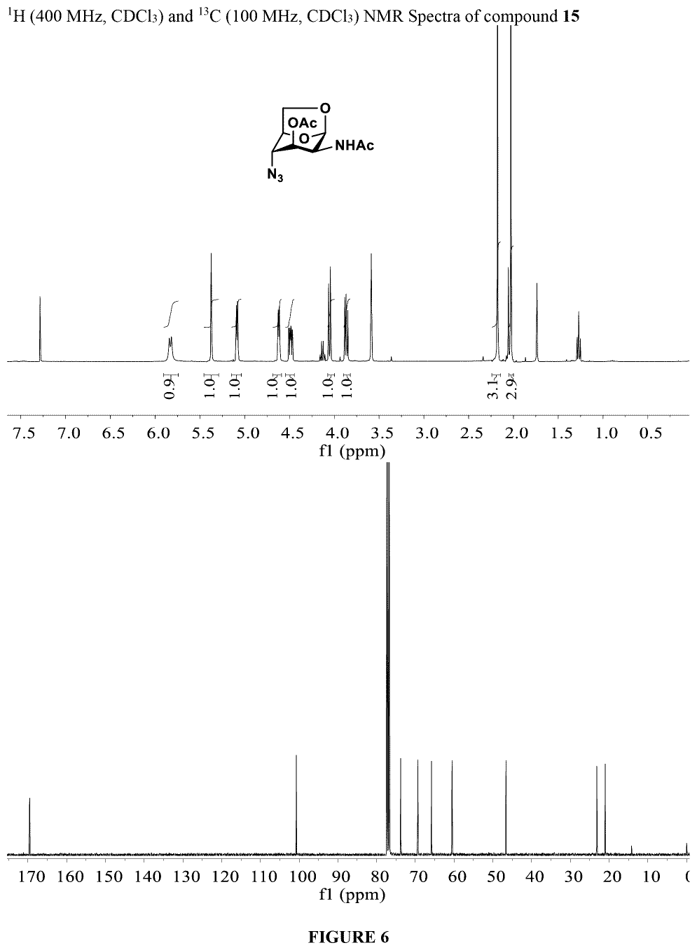

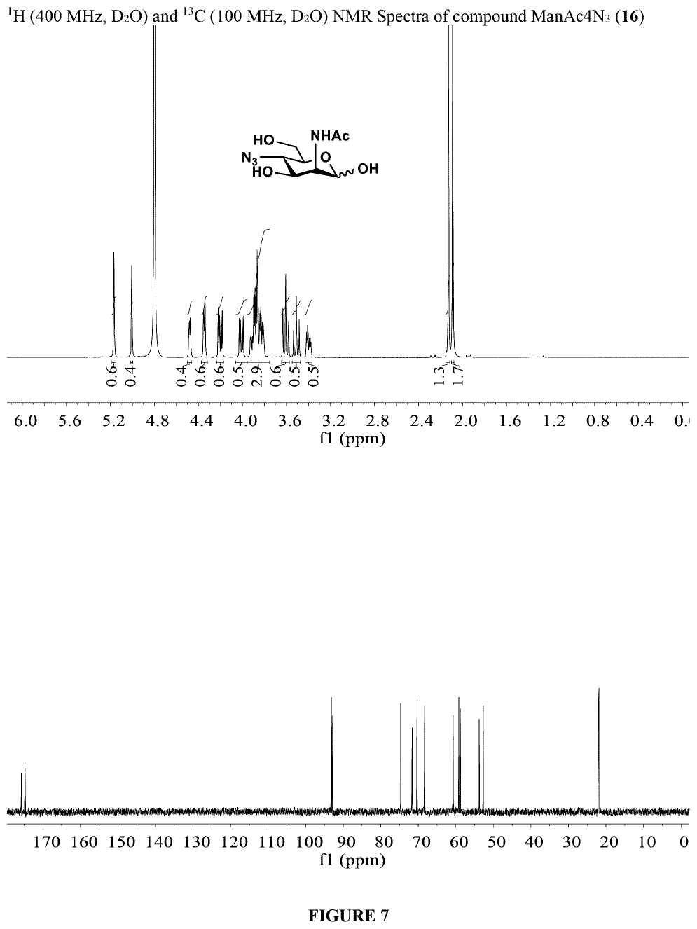

¹H-NMR and ¹³C-NMR spectra for compounds (15) and ManNAc4N₃ (16) are provided in FIGS. 6 and 7, respectively.

### Example 4: Chemical Synthesis of ManNAc5N₃, ManNAc5,6diN₃, and Neu5Ac8NAc 1. Chemical synthesis of ManNAc5N₃

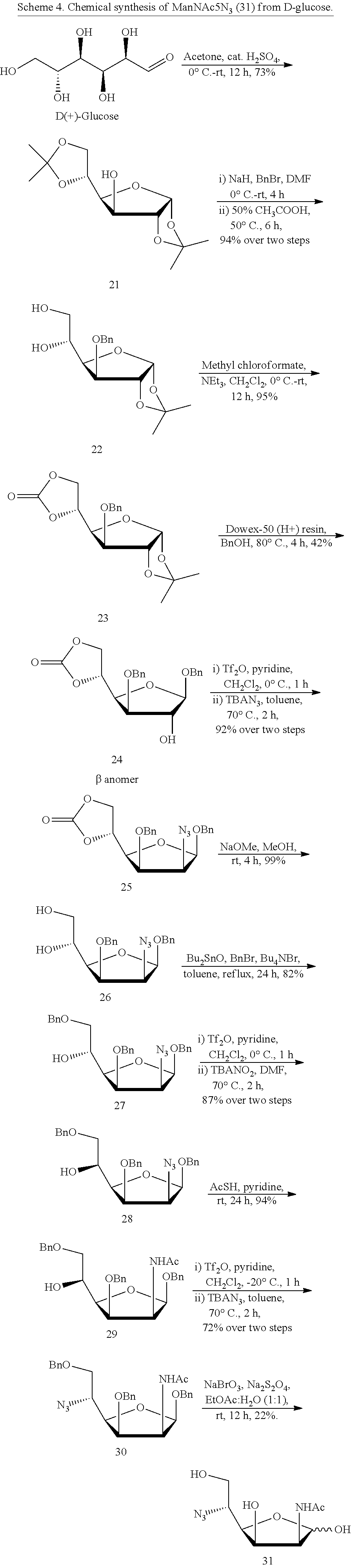

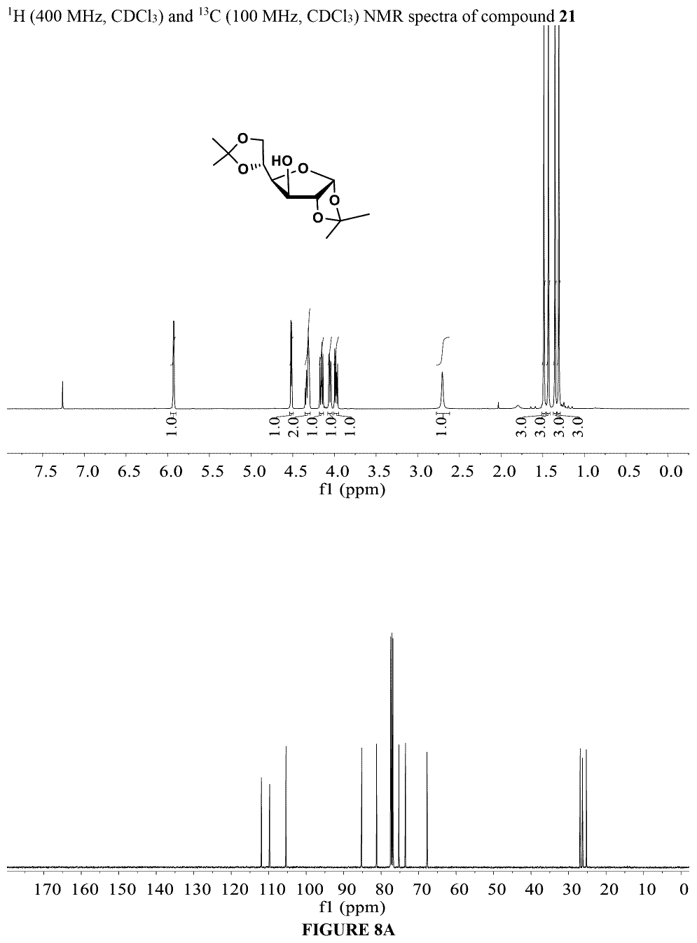

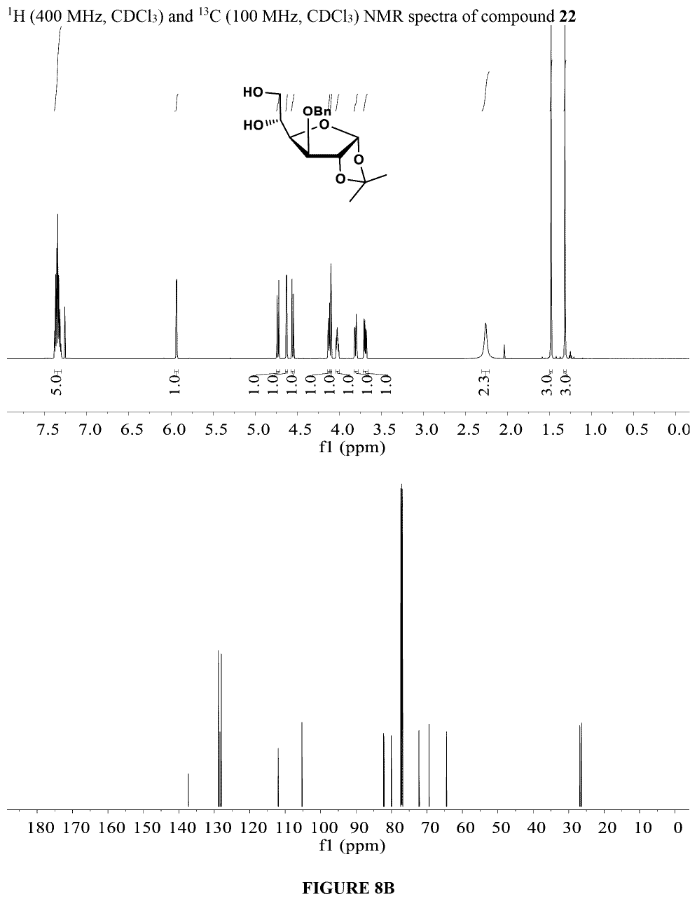

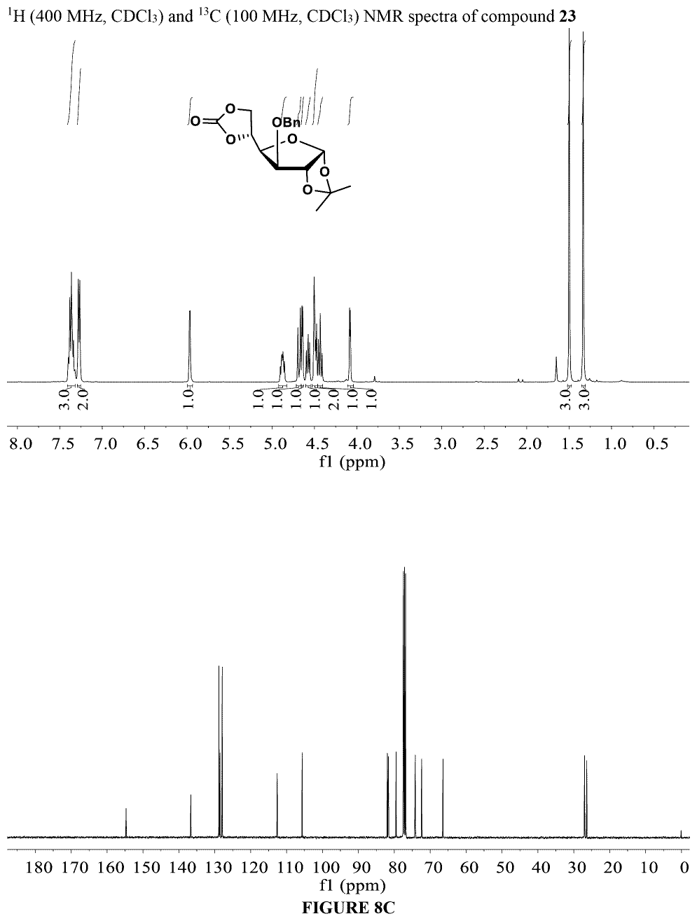

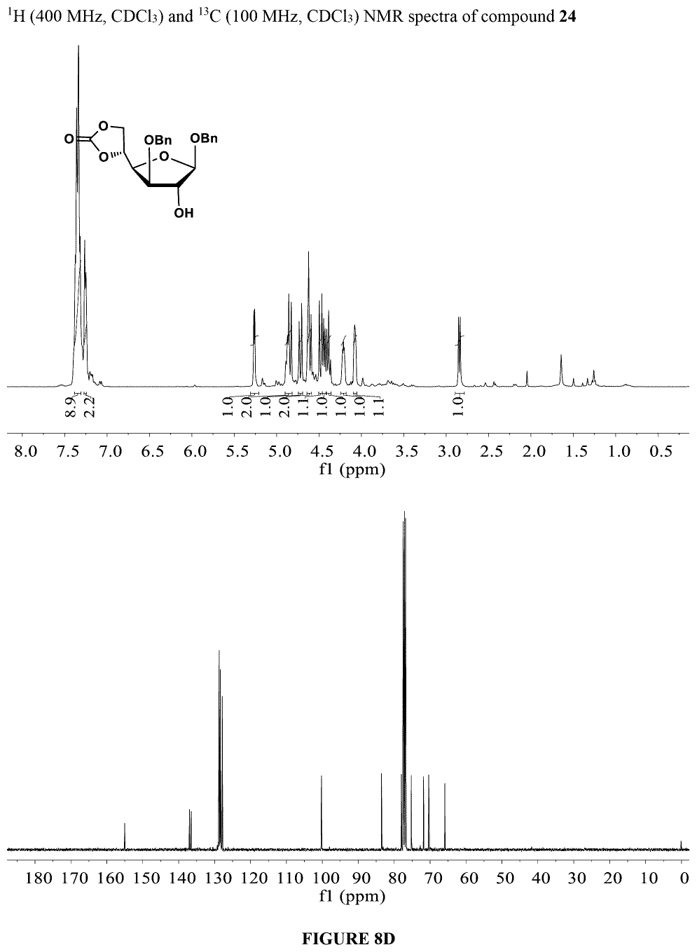

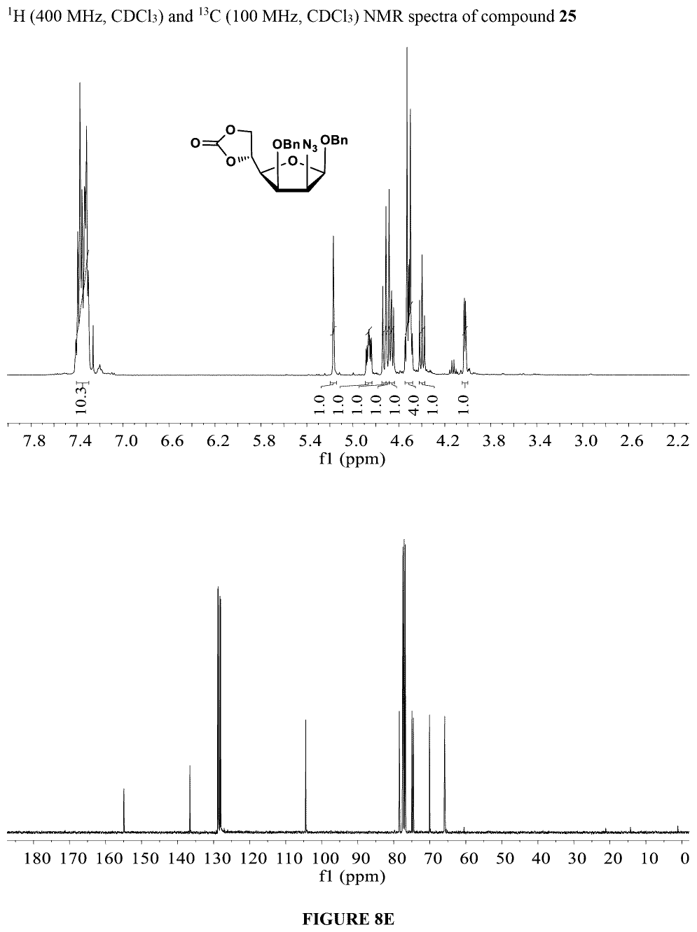

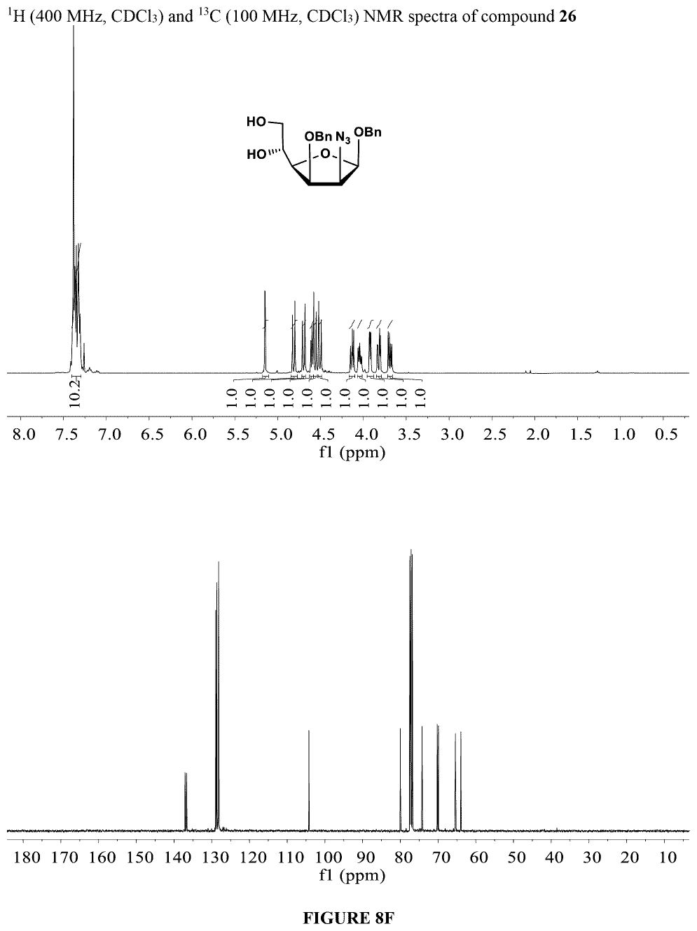

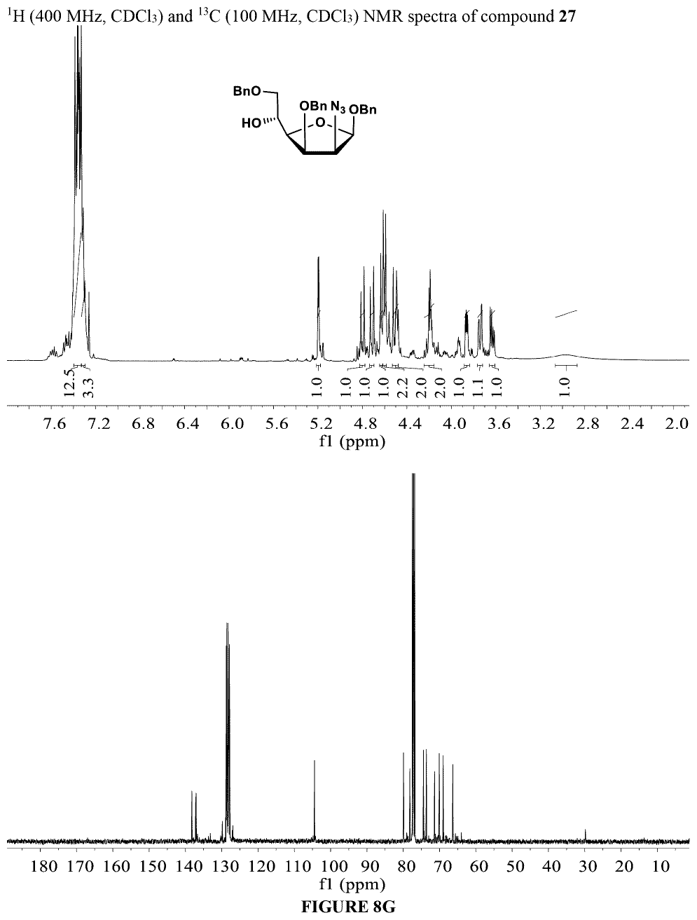

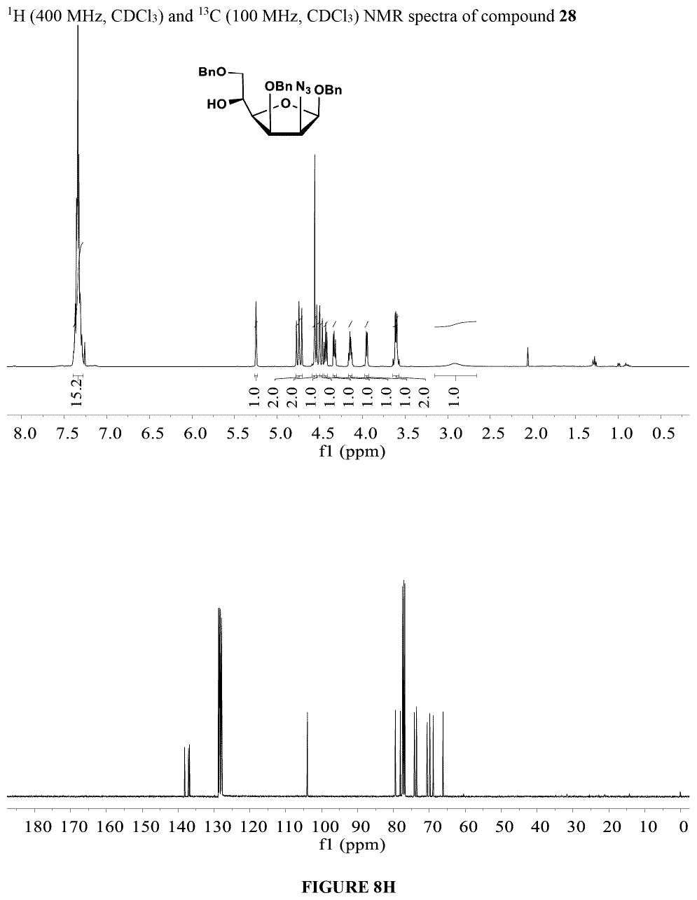

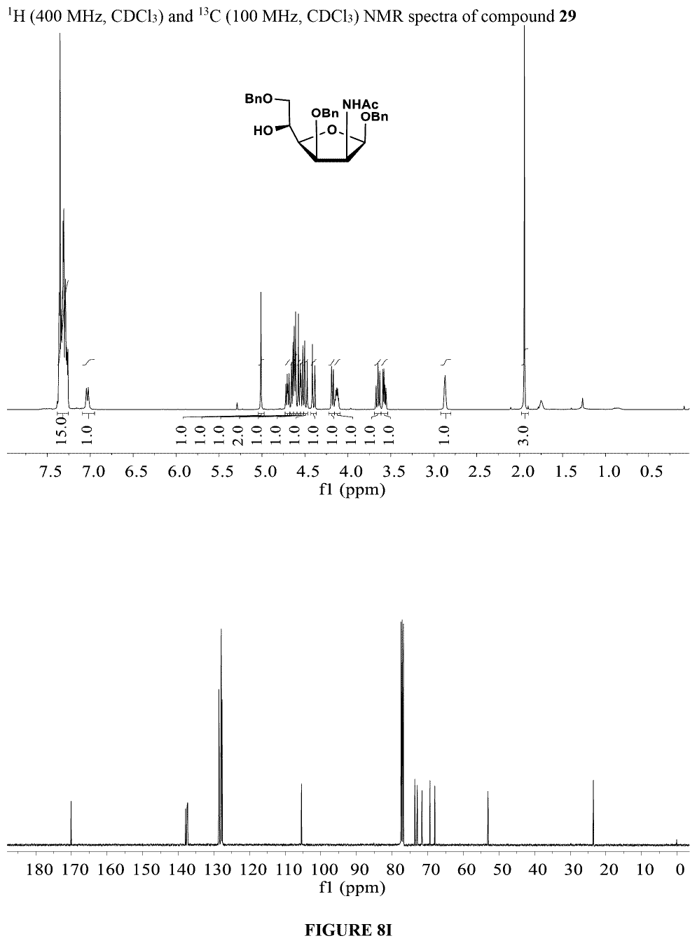

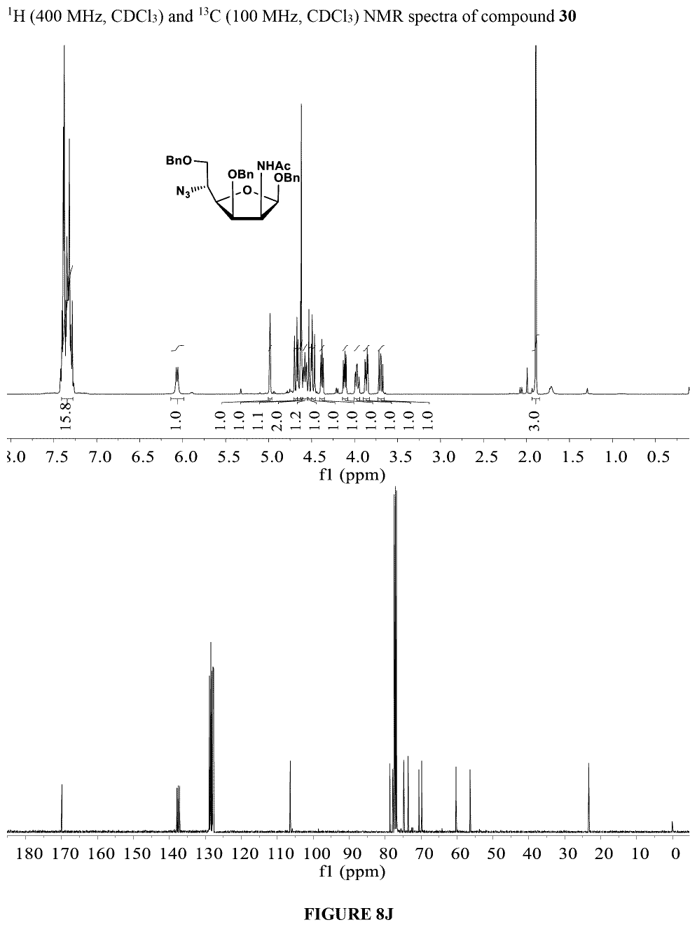

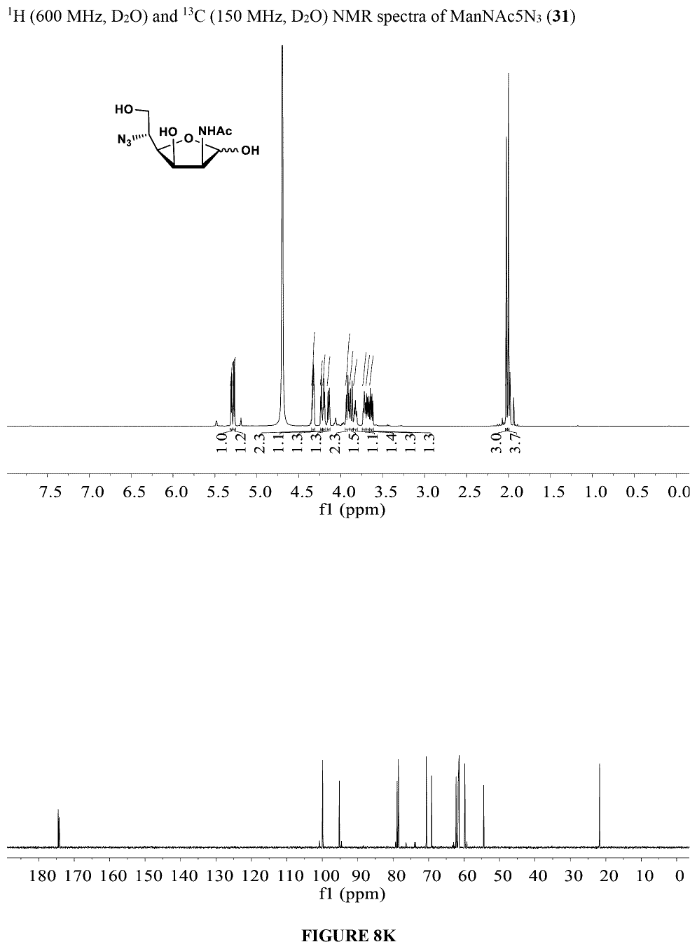

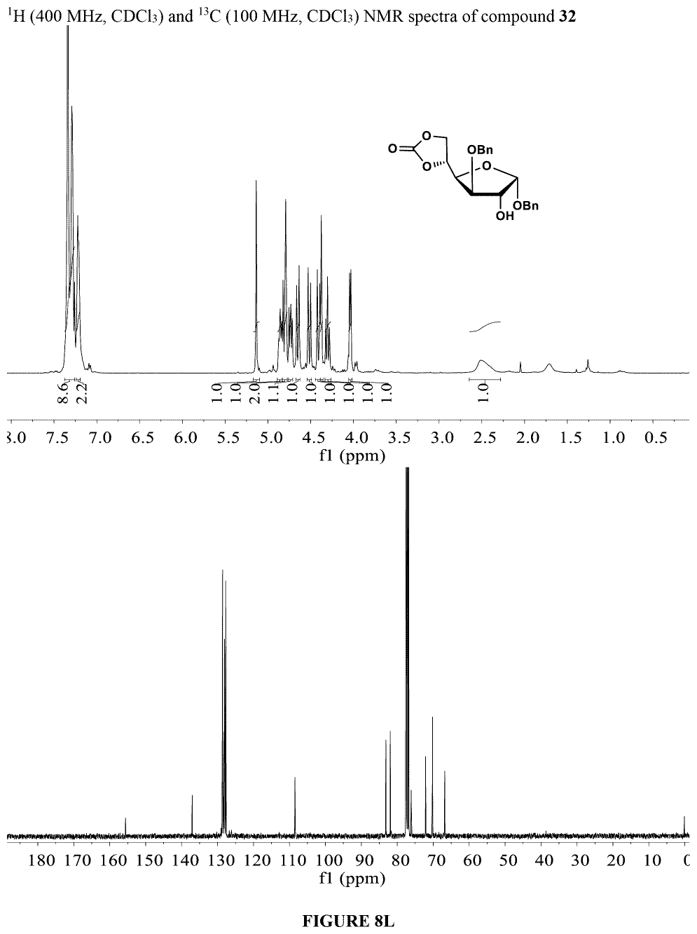

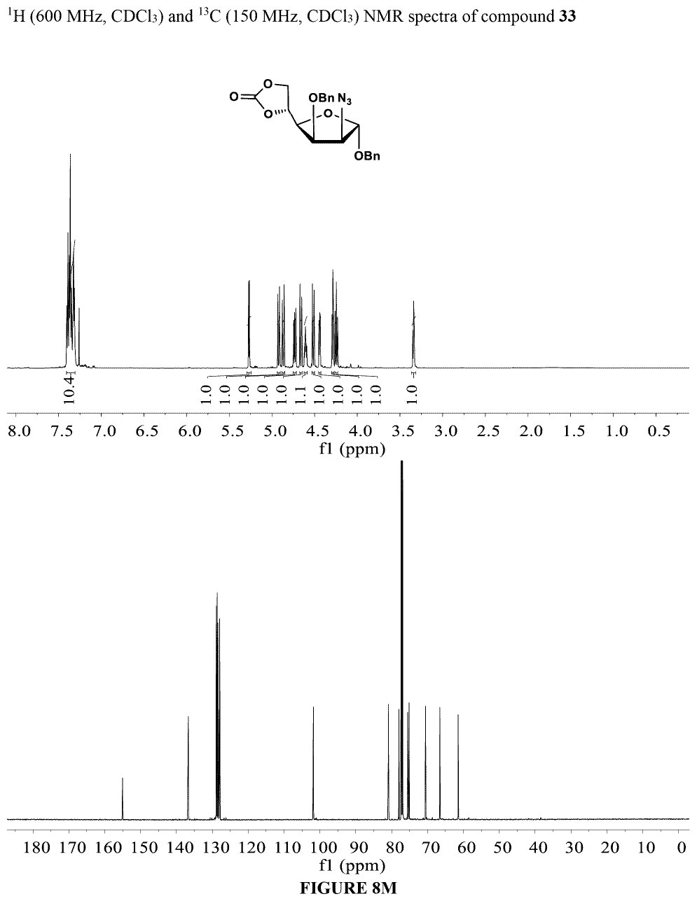

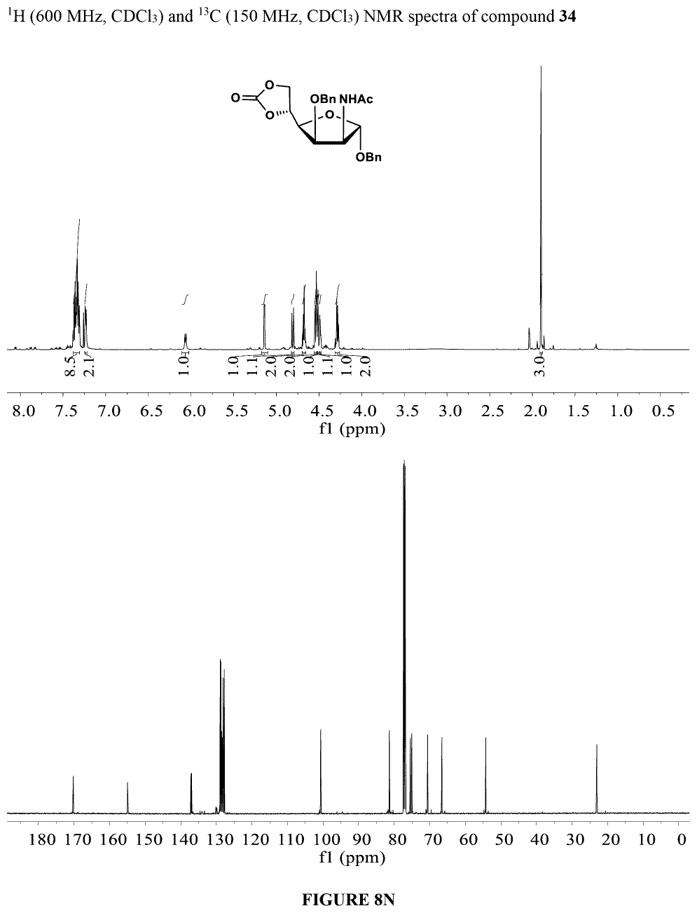

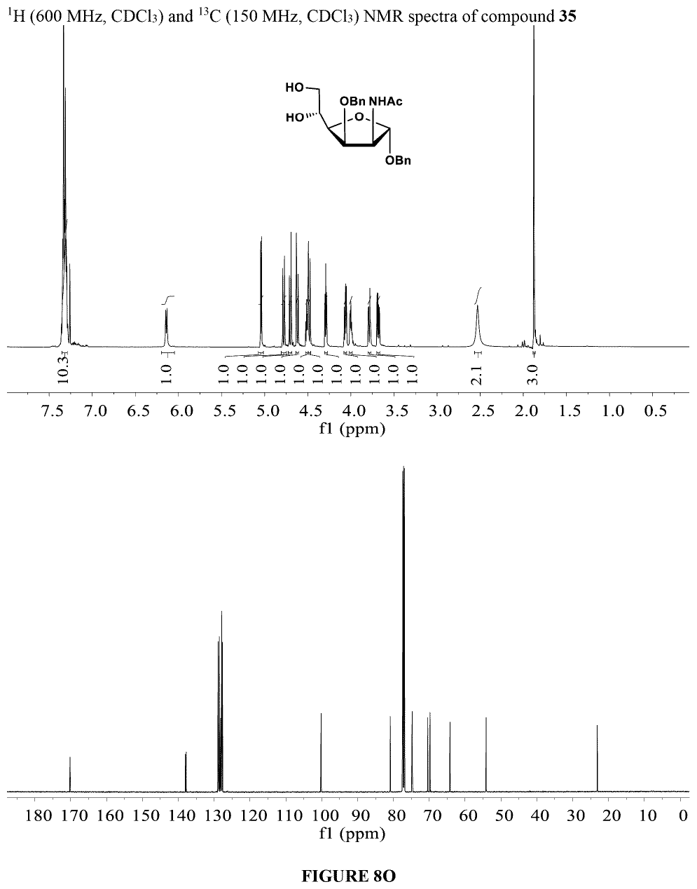

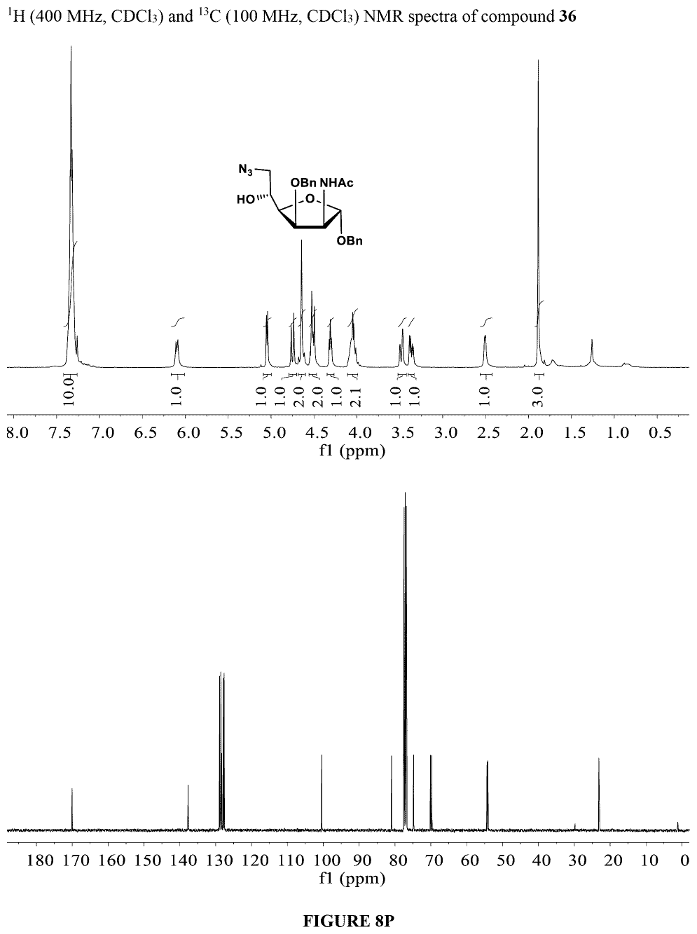

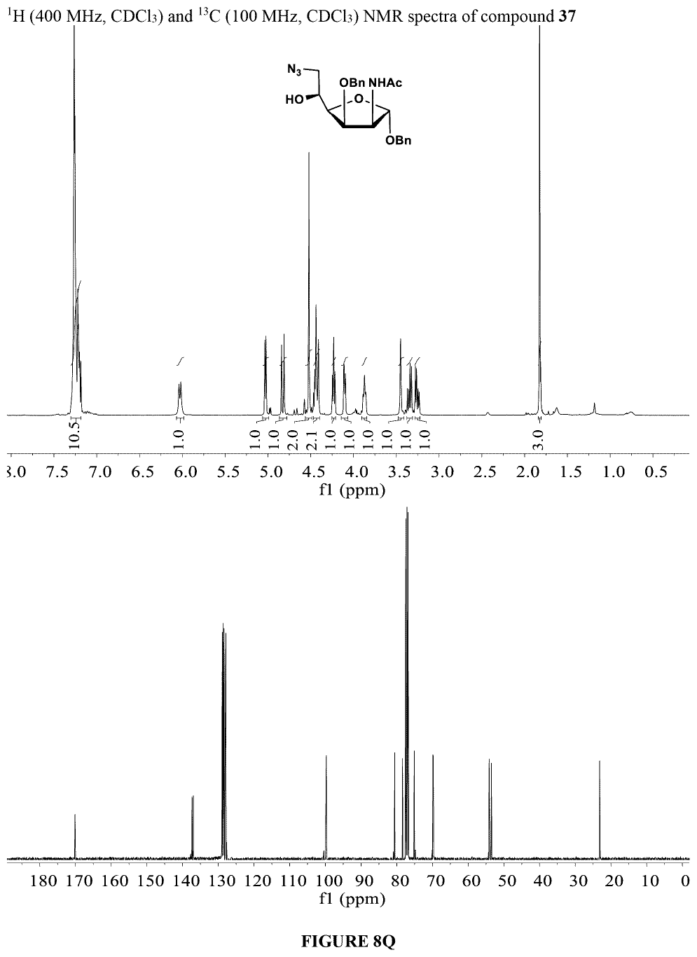

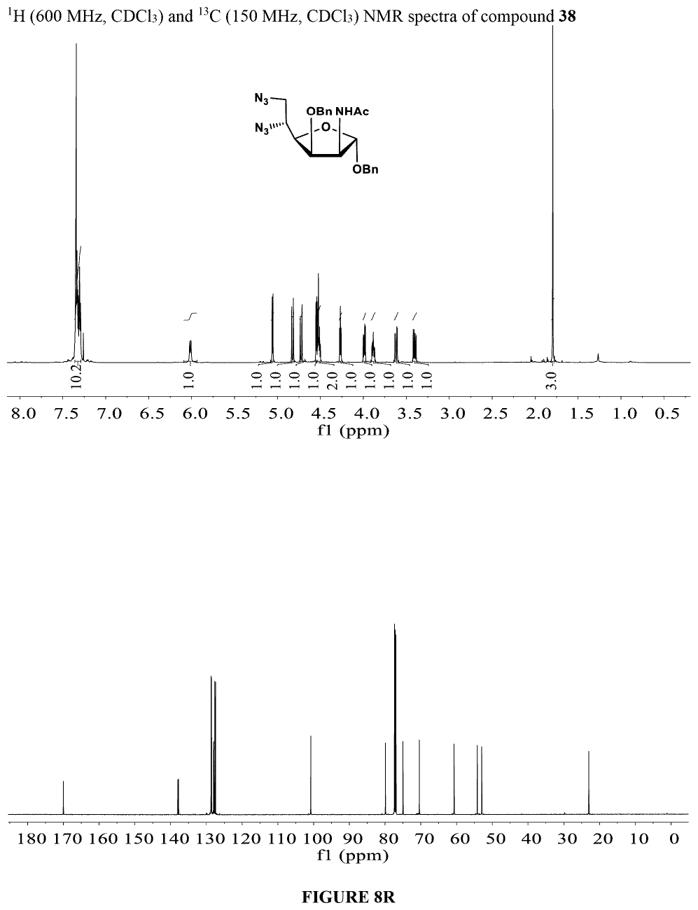

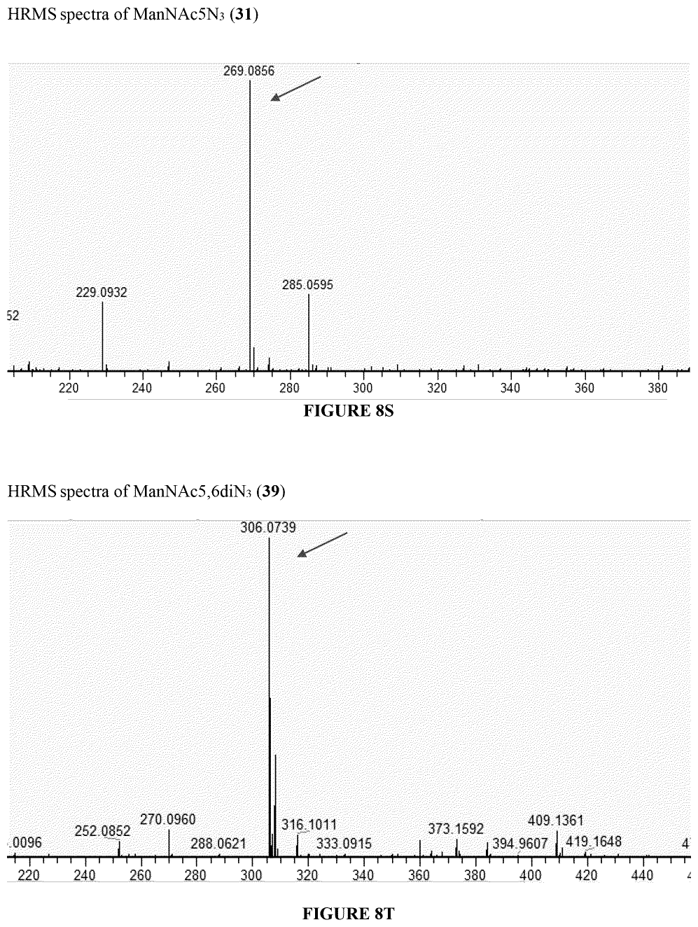

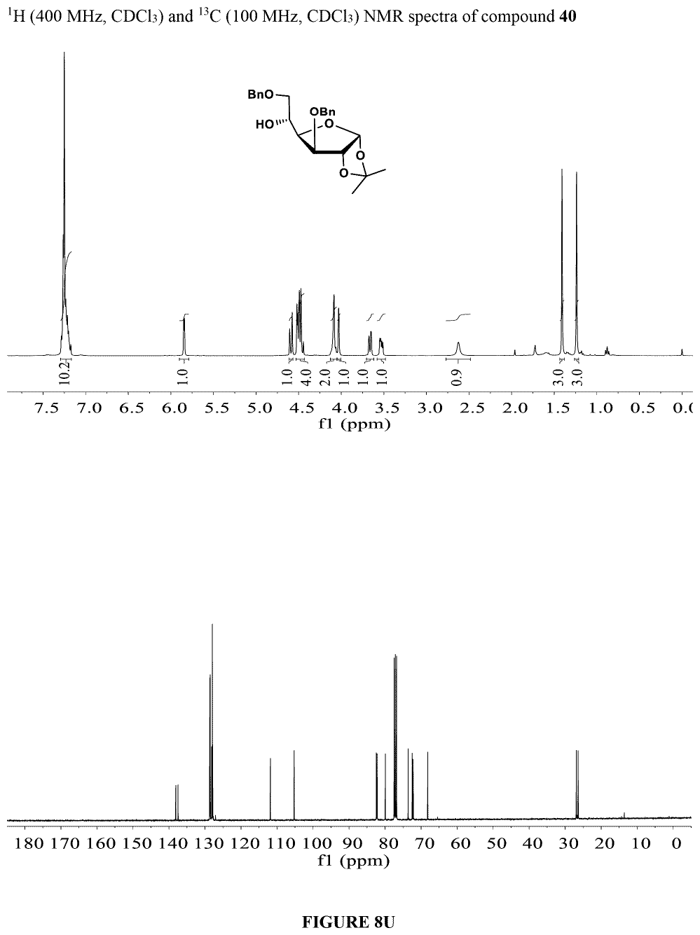

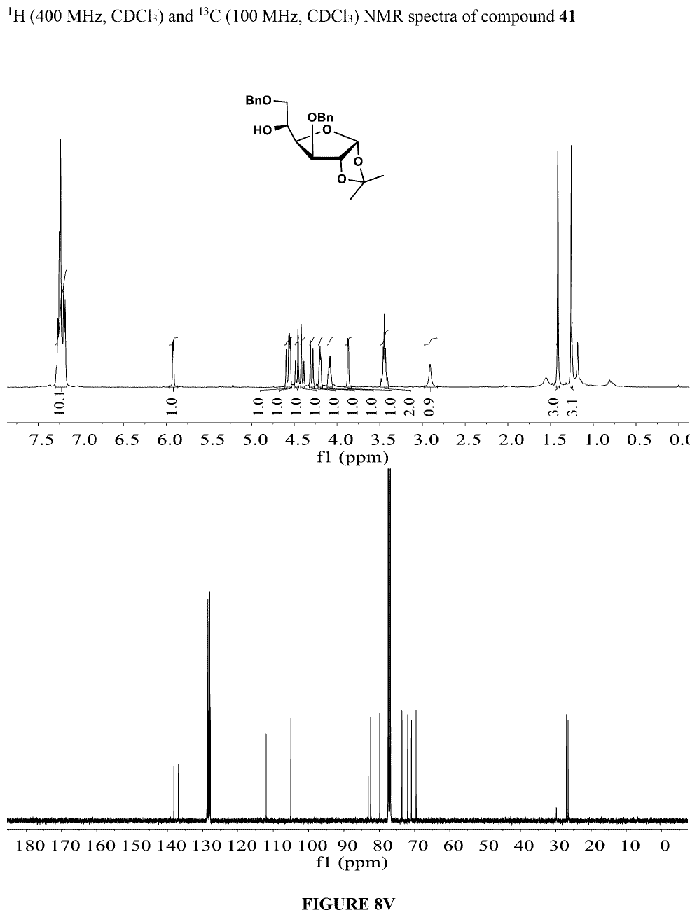

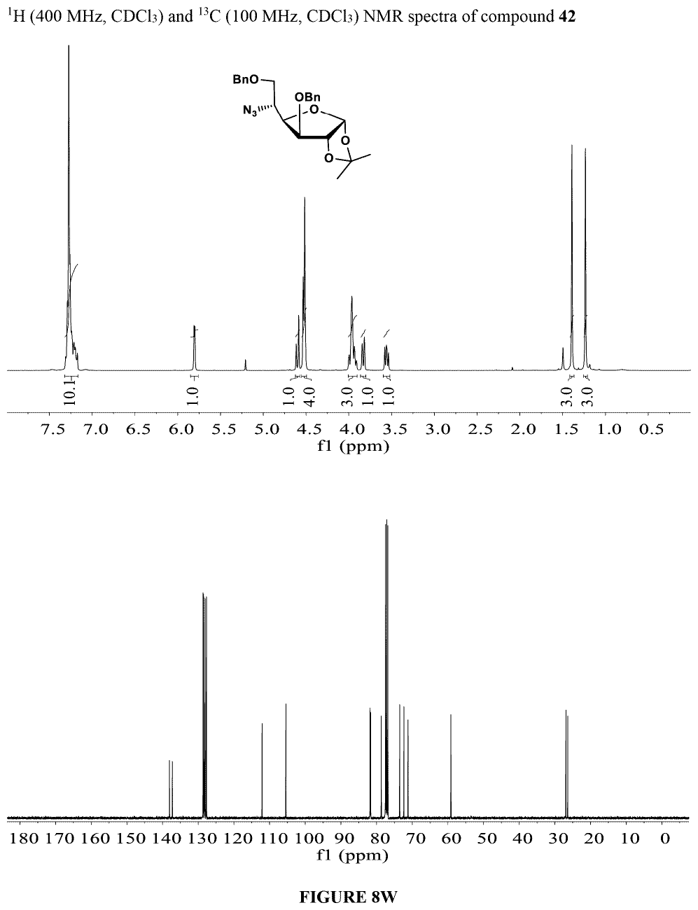

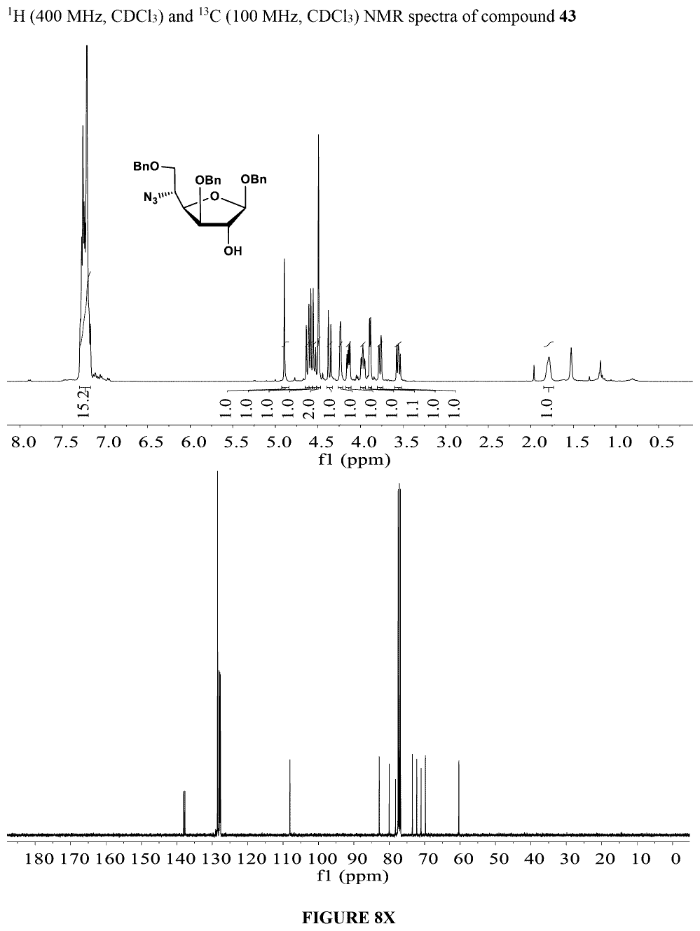

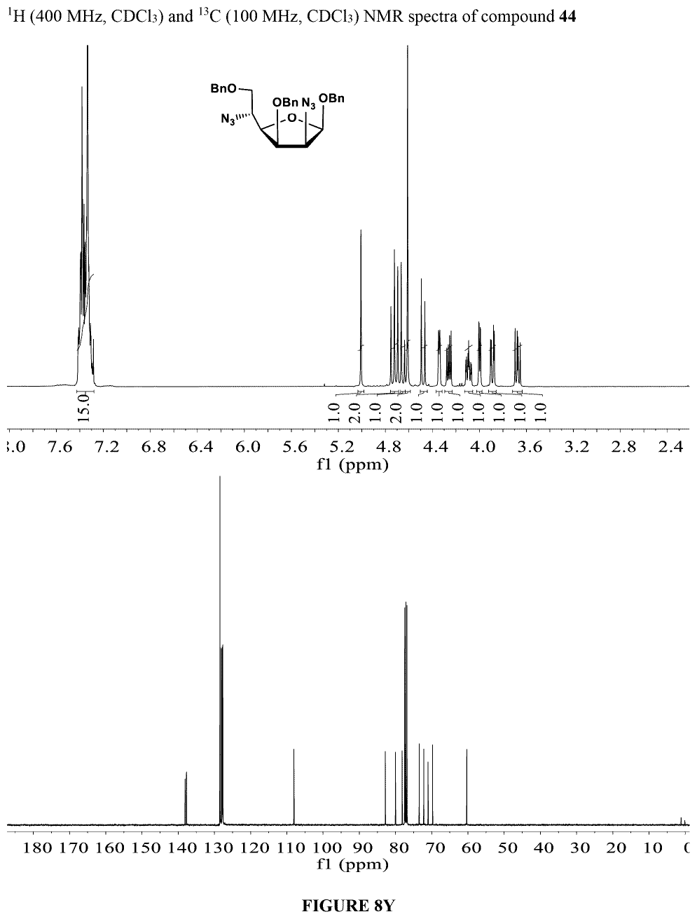

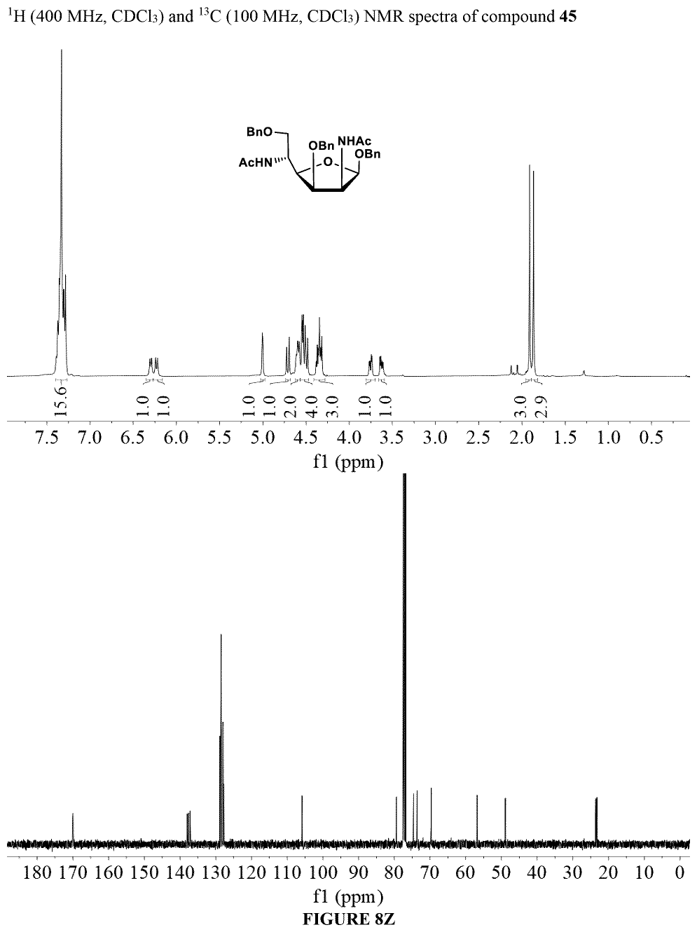

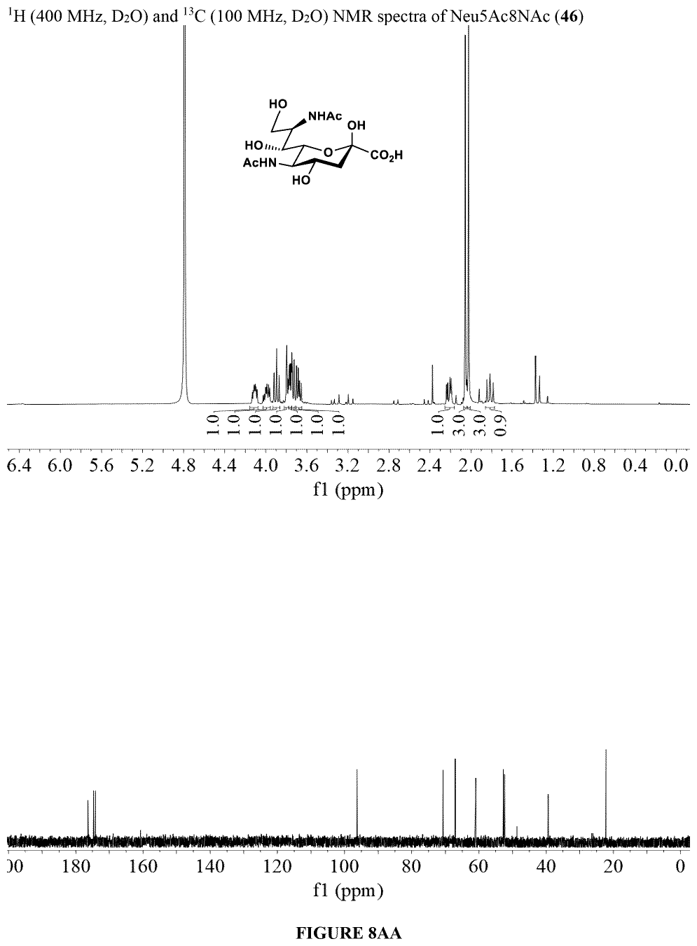

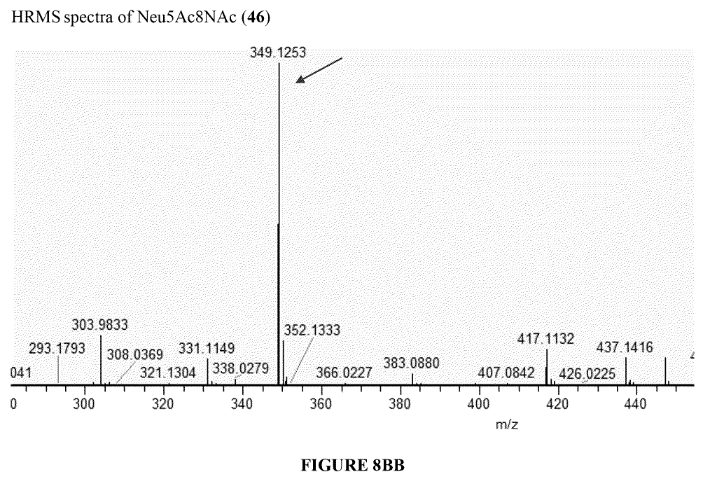

1,25,6-di-O-isopropylidene-α-D-glucofuranose (21): To a suspended solution of D-glucose (20 g, 111 mmol) in anhydrous acetone (800 mL), conc. H₂SO₄ (15 mL) was added at 0° C. under a nitrogen atmosphere. The reaction mixture was gradually warmed up to 25° C. and stirred for 12 hours. The reaction mixture was quenched with saturated KOH and filtered. The filtrate was concentrated under reduced pressure, re-dissolved in minimum amount of CH₂Cl₂ (50 mL), precipitated using excess hexane (500 mL) and filtered to get the compound 21 (21.09 g 73%) as white solid. ¹H NMR (400 MHz, CDCl₃) δ 5.93 (d, J=3.6 Hz, 1H), 4.52 (d, J=3.7 Hz, 1H), 4.36-4.26 (m, 2H), 4.15 (dd, J=8.7, 6.2 Hz, 1H), 4.05 (dd, J=7.7, 2.8 Hz, 1H), 3.98 (dd, J=8.6, 5.3 Hz, 1H), 2.70 (s, 1H), 1.49 (s, 3H), 1.43 (s, 3H), 1.35 (s, 3H), 1.31 (s, 3H); ¹³C NMR (100 MHz, CDCl₃) δ 111.9, 109.8, 105.4, 85.2, 81.3, 75.3, 73.5, 67.8, 27.0, 26.9, 26.3, 25.3. ¹H and ¹³C NMR spectra for compound 21 are provided in FIG. 8A.

3-O-Benzyl-1,2-O-isopropylidene-α-D-glucofuranose (22): To an ice-cooled solution of compound 21(15 g 57.63 mmol) in anhydrous DMF (100 mL), 60% NaH (3.46 g, 86.44 mmol) was added and the reaction mixture was stirred at 0° C. under a nitrogen atmosphere for 10 minutes. BnBr (8.2 mL, 69.15 mmol) was added drop-wisely to the reaction mixture followed by the addition of TBAI (2.13 g, 5.76 mmol) at 0° C. The reaction mixture was gradually warmed up to 25° C. and stirred for 4 hours. After complete consumption of the alcohol as indicated by TLC, the reaction mixture was poured into ice cold water with vigorous shaking and extracted with ethyl acetate (3×100 mL) and the combined EtOAc layer was washed with cold water and brine solution, dried over Na₂SO₄ and evaporated under reduced pressure. The crude residue of 3-O-benzyl-1,25,6-di-O-isopropylidene-α-D-glucofuranose was used directly for the next step reaction without any further purification.

A solution of 50% acetic acid-water mixture (100 mL) was added into the crude mixture and heated at 50° C. in an oil bath and stirred for 4 hours. After completion, as indicated by TLC, water (300 mL) was added into the reaction mixture and extracted with ethyl acetate (3×100 mL). The combined ethyl acetate layer was washed with saturated aqueous (sat. aq.) NaHCO₃ solution and brine solution. The organic layer was dried over Na₂SO₄ and concentrated under reduced pressure and the resulting crude residue was purified by silica gel column chromatography (n-hexane/EtOAc) to obtain the compound 22 (16.65 g, 94%) as colorless syrup. ¹H NMR (400 MHz, CDCl₃) δ 7.41-7.29 (m, 5H), 5.93 (d, J=3.8 Hz, 1H), 4.73 (d, J=11.7 Hz, 1H), 4.63 (d, J=3.8 Hz, 1H), 4.55 (d, J=11.7 Hz, 1H), 4.12 (dd, J=7.8, 3.3 Hz, 1H), 4.10 (d, J=3.2 Hz, 1H), 4.02 (ddd, J=7.9, 5.5, 3.5 Hz, 1H), 3.81 (dd, J=11.5, 3.5 Hz, 1H), 3.69 (dd, J=11.5, 5.5 Hz, 1H), 2.26 (s, 2H), 1.48 (s, 3H), 1.32 (s, 3H); ¹³C NMR (100 MHz, CDCl₃) δ 137.3, 128.9, 128.9, 128.4, 128.0, 128.0, 112.0, 105.3, 82.2, 82.1, 80.1, 72.3, 69.4, 64.5, 26.9, 26.4. ¹H and ¹³C NMR spectra for compound 22 are provided in FIG. 8B.

3-O-Benzyl-5,6-carbonate-1,2-O-isopropylidene-α-D-glucofuranose (23): Methyl chloroformate (20 mL, 257.78 mmol) was added to a solution of diol 2 (16 g, 51.56 mmol) and TEA (36 mL, 257.78 mmol) in CH₂Cl₂ (100 mL) at 0° C. The reaction mixture was stirred for 12 hours and the solvent was removed in vacuo. Et20 (200 mL) was added and the mixture was filtered. The solvent was removed in vacuo and the residue was purified on silica gel to give carbonate 23 (16.47 g, 95%) as colorless crystals. ¹H NMR (400 MHz, CDCl₃) δ 7.44-7.32 (m, 3H), 7.31-7.22 (m, 2H), 5.97 (d, J=3.6 Hz, 1H), 4.88 (ddd, J=8.7, 6.6, 4.7 Hz, 1H), 4.68 (d, J=11.6 Hz, 1H), 4.64 (d, J=3.6 Hz, 1H), 4.58 (dd, J=8.9, 6.5 Hz, 1H), 4.51-4.47 (m, 2H), 4.43 (t, J=8.6 Hz, 1H), 4.08 (d, J=3.5 Hz, 1H), 1.50 (s, 3H), 1.33 (s, 3H); ¹³C NMR (100 MHz, CDCl₃) δ 154.7, 136.7, 128.9, 128.9, 128.5, 127.9, 127.9, 112.6, 105.7, 81.9, 81.7, 79.5, 74.2, 72.4, 66.4, 27.0, 26.4. ¹H and ¹³C NMR spectra for compound 23 are provided in FIG. 8C.

Benzyl 3-O-benzyl-5,6-carbonate-β-D-glucofuranoside (24): A solution of 3-O-benzyl-5,6-carbonate 1,2-O-isopropylidene a-D-glucofuranose 3 (16 g, 47.57 mmol) in benzyl alcohol (100 mL) was stirred for 4 hours at 80° C. in the presence of Dowex-50 H⁺ ion exchange resin (15 g). The filtered solution was purified on silica gel column and washed first with hexane:ethyl acetate (19:1, v/v) to remove most of the benzyl alcohol, then washed with hexane:ethyl acetate (10:1 to 1:1, v/v) to give P-anomer 24 (7.72 g, 42%) as yellow syrup ¹H NMR (400 MHz, CDCl₃) δ 7.40-7.32 (m, 8H), 7.28-7.22 (m, 2H), 5.26 (d, J=4.6 Hz, 1H), 4.90-4.82 (m, 2H), 4.72 (d, J=11.7 Hz, 1H), 4.64-4.59 (m, 2H), 4.48 (d, J=11.8 Hz, 1H), 4.44 (d, J=7.1 Hz, 1H), 4.39 (t, J=8.7 Hz, 1H), 4.21 (dt, J=7.9, 3.8 Hz, 1H), 4.08 (dd, J=6.3, 3.2 Hz, 1H), 2.84 (d, J=7.9 Hz, 1H); ¹³C NMR (100 MHz, CDCl₃) δ 155.0, 137.0, 136.5, 128.8, 128.8, 128.7, 128.7, 128.7, 128.4, 128.4, 128.3, 127.9, 127.9, 100.3, 83.5, 78.0, 77.4, 75.2, 71.8, 70.4, 65.9. ¹H and ¹³C NMR spectra for compound 24 are provided in FIG. 8D.

Benzyl 2-azido-3-O-benzyl-5,6-carbonate-2-deoxy-β-D-mannofuranoside (25): To a solution of compound 24 (7.60 g, 19.67 mmol) in CH₂Cl₂ (60 mL) and pyridine (15 mL), trifluoromethanesulfonic anhydride (9.9 mL, 59.01 mmol) was added drop-wisely at 0° C. with stirring. After 1 hour, the mixture was diluted with dichloromethane (50 mL) and washed successively with cold HCl solution (1 M), saturated NaHCO₃ and brine, dried over Na₂SO₄, filtrated and concentrated to give crude triflate ester. The crude triflate ester was used directly in the next step without further purification. To the solution of crude triflate ester in dry toluene (80 mL), tetrabutylammonium azide (11.19 g, 39.35 mmol) was added and the reaction mixture was heated at 70° C. with continuous stirring. After 2 hours, the reaction was stopped, solvent was removed and the condensed mixture was diluted with 75 mL of CH₂Cl₂. The organic layer was washed with brine solution, dried over anhydrous Na₂SO₄. After filtration, the solvent was removed under reduced pressure and the product was purified by silica gel chromatography using hexane:ethylacetate (4:1 v/v) to produce compound 25 (7.45 g, 92% over 2 steps) as a white solid. ¹H NMR (400 MHz, CDCl₃) δ 7.41-7.29 (m, 1OH), 5.17 (d, J=1.6 Hz, 1H), 4.86 (ddd, J=8.4, 6.5, 3.4 Hz, 1H), 4.73 (d, J=10.9 Hz, 1H), 4.70 (d, J=10.9 Hz, 1H), 4.65 (dd, J=6.6, 2.3 Hz, 1H), 4.55-4.47 (m, 4H), 4.40 (t, J=8.7 Hz, 1H), 4.03 (dd, J=5.2, 1.6 Hz, 1H); ¹³C NMR (100 MHz, CDCl₃) δ 154.9, 136.6, 136.6, 128.9, 128.9, 128.7, 128.7, 128.6, 128.3, 128.3, 128.3, 128.1, 128.1, 104.4, 78.5, 77.6, 74.9, 74.6, 70.1, 66.0, 65.9. ¹H and ¹³C NMR spectra for compound 25 are provided in FIG. 8E.

Benzyl 2-azido-3-O-benzyl-2-deoxy-β-D-mannofuranoside (26): To a solution of compound 25 (7.3 g, 17.74 mmol) in dry methanol (70 mL), 30% sodium methoxide in methanol (1 mL) was added at room temperature. After 3 hours, the reaction mixture was neutralized with Dowex 50W (H⁺), filtered and concentrated under reduced pressure and purified using flash chromatography hexane:ethyl acetate (2:1 to 1:1, v/v) to give compound 26 (6.77 g, 99%) as white solid. ¹H NMR (400 MHz, CDCl₃) δ 7.41-7.29 (m, 1OH), 5.15 (d, J=1.6 Hz, 1H), 4.81 (d, J=11.2 Hz, 1H), 4.70 (d, J=11.7 Hz, 1H), 4.60 (dd, J=6.5, 5.4 Hz, 1H), 4.57 (d, J=11.3 Hz, 1H), 4.51 (d, J=11.8 Hz, 1H), 4.13 (dd, J=8.9, 6.3 Hz, 1H), 4.04 (ddd, J=8.8, 5.2, 3.3 Hz, 1H), 3.92 (dd, J=5.2, 1.7 Hz, 1H), 3.82 (dd, J=11.5, 3.3 Hz, 1H), 3.69 (dd, J=11.5, 5.2 Hz, 1H); ¹³C NMR (100 MHz, CDCl₃) δ 137.1, 136.7, 128.9, 128.9, 128.7, 128.6, 128.6, 128.2, 128.2, 128.2, 128.2, 128.2, 104.3, 80.0, 77.3, 74.3, 70.2, 69.9, 65.4, 64.0. ¹H and ¹³C NMR spectra for compound 26 are provided in FIG. 8F.

Benzyl 2-azido-3,6-di-O-benzyl-2-deoxy-β-D-mannofuranoside (27): To the suspended mixture of compound 26 (6.70 g, 17.38 mmol) and dibutyltin oxide (6.49 g, 26.08 mmol) in dry toluene (100 mL) were heated under reflux for 3 hours. Approximately 50 mL solvent was azeotropicly removed using a Dean-stark apparatus under nitrogen atmosphere. After that tetrabutylammonium bromide (280 mg, 0.870 mmol) and benzyl bromide (2.68 mL, 22.60 mmol) were added into the reaction mixture and heated under reflux for 24 hours. The solvent was evaporated, the residue was diluted with ethyl acetate, washed successively with 10% aqueous KF solution and brine, dried over Na₂SO₄, and concentrated. The residue was purified by flash chromatography with hexane:ethylacetate (10:1 to 5:1, v/v) to obtain compound 27 (6.78 g, 82%) as a syrup. ¹H NMR (400 MHz, CDCl₃) δ 7.40-7.35 (m, 12H), 7.34-7.31 (m, 3H), 5.20 (d, J=2.6 Hz, 1H), 4.80 (d, J=11.1 Hz, 1H), 4.71 (d, J=11.7 Hz, 1H), 4.64 (d, J=3.0 Hz, 1H), 4.60 (dd, J=8.6, 2.3 Hz, 2H), 4.53-4.47 (m, 2H), 4.23-4.16 (m, 2H), 3.86 (dd, J=5.1, 2.6 Hz, 1H), 3.74 (dd, J=9.9, 2.4 Hz, 1H), 3.63 (dd, J=10.0, 4.9 Hz, 1H), 2.96 (s, 1H); ¹³C NMR (100 MHz, CDCl₃) δ 138.3, 137.2, 137.1, 128.8, 128.8, 128.6, 128.6, 128.5, 128.5, 128.4, 128.3, 128.3, 128.2, 128.2, 128.1, 127.9, 127.9, 127.8, 104.5, 79.9, 78.2, 74.5, 73.6, 71.4, 70.1, 69.0, 66.4. ¹H and ¹³C NMR spectra for compound 27 are provided in FIG. 8G.

Benzyl 2-azido-3,6-di-O-benzyl-2-deoxy-β-L-gulofuranoside (28): To a solution of compound 27 (6.7 g, 14.09 mmol) in CH₂Cl₂ (50 mL) in pyridine (12 mL), trifluoromethanesulfonic anhydride (7.10 mL, 42.27 mmol) was added drop-wisely at 0° C. with stirring. After 1 hour, the mixture was diluted with dichloromethane (50 mL) and washed successively with cold 1 M HCl, saturated NaHCO₃ and brine, dried over Na₂SO₄, filtrated and concentrated to give crude triflate ester which was used directly in the next step without further purification. The crude triflate ester was first re-dissolved in dry DMF (80 mL) and divided into two equal portions. Each portion having 40 mL of triflate ester solution was used separately for the next step reaction. Tetrabutylammonium nitrite (6.1 g, 21.13 mmol) was added and the reaction mixture was heated at 70° C. with continuous stirring. After 2 hours, the reaction was stopped, solvent was removed and the condensed mixture was diluted with 75 mL of CH₂Cl₂. The organic layer was washed with brine solution, dried over anhydrous Na₂SO₄. After filtration, the solvent was removed under reduced pressure and the product was purified by silica gel chromatography using hexane:ethylacetate (4:1 v/v) to produce protected L-gulose derivative 28 (5.83 g, 87% over 2 steps) as a colorless syrup. ¹H NMR (400 MHz, CDCl₃) δ 7.40-7.27 (m, 15H), 5.25 (d, J=1.8 Hz, 1H), 4.74 (dd, J=13.2, 11.5 Hz, 2H), 4.56 (s, 2H), 4.52 (d, J=11.7 Hz, 1H), 4.49 (d, J=11.5 Hz, 1H), 4.46-4.40 (m, 1H), 4.33 (dd, J=6.4, 3.6 Hz, 1H), 4.14 (td,J=5.8, 3.5 Hz, 1H), 3.95 (dd, J=5.4, 1.8 Hz, 1H), 3.60 (dt, J=6.5, 3.4 Hz, 2H), 2.91 (s, 1H); ¹³C NMR (100 MHz, CDCl₃) δ 138.2, 137.1, 136.8, 128.7, 128.7, 128.6, 128.6, 128.5, 128.5, 128.4, 128.2, 128.2, 128.1, 128.1, 128.1, 127.9, 127.9, 127.8, 104.0, 79.5, 78.1, 74.2, 73.6, 70.6, 69.9, 69.0, 66.2. ¹H and ¹³C NMR spectra for compound 28 are provided in FIG. 8H.

Benzyl 2-N-acetyl-3,6-di-O-benzyl-2-deoxy-β-L-gulofuranoside (29): To a solution of compound 28 (5.7 g, 11.99 mmol) in pyridine (50 mL), thioacetic acid (8.5 mL, 119.86 mmol) was added under argon at room temperature and the reaction mixture was stirred for 24 hours. The product was purified by silica gel chromatography using hexane:ethylacetate (4:1 to 2:1 v/v) as an eluent to produce compound 29 (5.54 g, 94%) as a white solid. ¹H NMR (400 MHz, CDCl₃) δ 7.39-7.25 (m, 15H), 7.03 (d, J=9.0 Hz, 1H), 5.01 (s, 1H), 4.70 (dd, J=9.1, 6.2 Hz, 1H), 4.65 (d, J=6.8 Hz, 1H), 4.61 (dd, J=6.6, 2.5 Hz, 2H), 4.57 (d, J=9.1 Hz, 1H), 4.54 (d, J=8.7 Hz, 1H), 4.49 (d, J=11.8 Hz, 1H), 4.40 (d, J=11.5 Hz, 1H), 4.18 (dd, J=8.3, 1.3 Hz, 1H), 4.13 (dt, J=8.1, 4.2 Hz, 1H), 3.65 (dd, J=9.5, 8.2 Hz, 1H), 3.57 (dd, J=9.5, 4.8 Hz, 1H), 2.87 (dd, J=4.2, 2.5 Hz, 1H), 1.94 (s, 3H); ¹³C NMR (100 MHz, CDCl₃) δ 170.1, 138.0, 137.5, 137.4, 128.6, 128.6, 128.6, 128.6, 128.5, 128.5, 128.1, 128.0, 128.0, 128.0, 127.9, 127.9, 127.9, 127.7, 127.7, 105.5, 77.4, 77.1, 73.6, 72.9, 71.6, 69.4, 68.0, 53.1, 23.5. ¹H and ¹³C NMR spectra for compound 29 are provided in FIG. 81.

Benzyl 5-azido-3,6-di-O-benzyl-5-deoxy-β-D-N-acetylmannosamine (30): To a solution of compound 29 (3.0 g, 6.10 mmol) in CH₂Cl₂ (30 mL) and pyridine (5 mL), trifluoromethanesulfonic anhydride (3.1 mL, 18.31 mmol) was added drop-wisely at −20° C. with stirring. After 1 hour, the mixture was diluted with dichloromethane (30 mL) and washed successively with cold HCl solution (1 M), saturated NaHCO₃ and brine, dried over Na₂SO₄, filtrated and concentrated to give crude triflate ester. The crude triflate ester was used directly in the next step without further purification. To the solution of crude triflate ester in dry toluene (40 mL), tetrabutylammonium azide (5.21 g, 18.33 mmol) was added and the reaction mixture was heated at 70° C. with continuous stirring. After 2 hours, the reaction was stopped, solvent was removed and the condensed mixture was diluted with 30 mL of CH₂Cl₂. The organic layer was washed with brine solution, dried over anhydrous Na₂SO₄. After filtration, the solvent was removed under reduced pressure and the product was purified by silica gel chromatography using hexane:ethylacetate (3:1 v/v) to produce compound 30 (2.27 g, 72% over 2 steps) as a white solid. ¹H NMR (400 MHz, CDCl₃) δ 7.40-7.28 (m, 15H), 6.07 (d, J=8.0 Hz, 1H), 4.99 (d, J=2.2 Hz, 1H), 4.69 (d, J=11.3 Hz, 1H), 4.64 (d, J=12.0 Hz, 1H), 4.62 (s, 2H), 4.58 (ddd, J=8.4, 6.2, 2.4 Hz, 1H), 4.52 (d, J=12.0 Hz, 1H), 4.48 (d, J=11.3 Hz, 1H), 4.38 (dd, J=6.1, 5.0 Hz, 1H), 4.11 (dd, J=8.5, 5.0 Hz, 1H), 4.00-3.94 (m, 1H), 3.86 (dd, J=10.2, 2.9 Hz, 1H), 3.69 (dd, J=10.2, 7.4 Hz, 1H), 1.89 (s, 3H); ¹³C NMR (100 MHz, CDCl₃) δ 169.9, 137.9, 137.6, 137.2, 128.9, 128.9, 128.6, 128.6, 128.5, 128.5, 128.5, 128.2, 128.2, 127.9, 127.9, 127.9, 127.8, 127.7, 127.7, 106.4, 78.6, 77.9, 74.8, 73.6, 70.6, 69.8, 60.2, 56.4, 23.3. ¹H and ¹³C NMR spectra for compound 30 are provided in FIG. 8J.

ManNAc5N₃ (α:β=1.2:1.0) (31): To a solution of compound 30 (1.0 g, 1.94 mmol) in EtOAc (10 mL), a solution of NaBrO₃ (1.75 g, 11.61 mmol) in water (5 mL) was added. A solution of Na₂S₂O₄ (1.69 g, 7.2 mmol) in water (5 mL) was then added over 25 minutes and the mixture was stirred vigorously at ambient temperature for 12 hours. After that the mixture was quenched with 10% sodium thiosulphate (2 mL) and neutralized with 1 M NaOH solution. The water layer was separated from organic layer and filtered to remove the white precipitate formed during the neutralization process. The filtrate was evaporated and the crude product was purified using Bio-Gel P-2 Gel (Bio-Rad) column as well as C18 reverse phase column to get the ManNAc5N₃ β1) as a α/β mixture (105 mg, 22%) as white solid. ¹H NMR (600 MHz, D₂O) δ 5.40 (d, J=5.2 Hz, 1H), 5.37 (d, J=6.2 Hz, 1H), 4.44-4.41 (m, 2H), 4.32 (t, J=5.1 Hz, 1H), 4.29 (dd, J=6.2, 4.5 Hz, 1H), 4.24 (dd, J=9.6, 2.7 Hz, 1H), 4.03-3.99 (m, 2H), 3.97 (dd, J=12.0, 2.8 Hz, 1H), 3.92 (ddd, J=9.6, 6.4, 2.6 Hz, 1H), 3.81 (ddd, J=9.4, 6.6, 2.6 Hz, 1H), 3.77 (dd, J=12.0, 6.5 Hz, 1H), 3.73 (dd, J=12.0, 6.7 Hz, 1H), 2.12 (s, 3H), 2.09 (s, 3H); ¹³C NMR (150 MHz, D₂O) δ 174.5, 174.3, 99.9, 95.2, 78.9, 78.5, 70.7, 69.2, 62.2, 61.7, 61.5, 61.4, 59.8, 54.5, 21.8, 21.7; HRMS (ESI-Orbitrap) m/z: [M+Na]⁺ Calcd for C₈H₁₄N₄O₅Na 269.0862; found 269.0858. ¹H and ¹³C NMR spectra for compound 31 are provided in FIG. 8K. HRMS spectrum for compound 31 is provided in FIG. 8S.

### 2. Chemical synthesis of ManNAc5,6diN₃ (39)

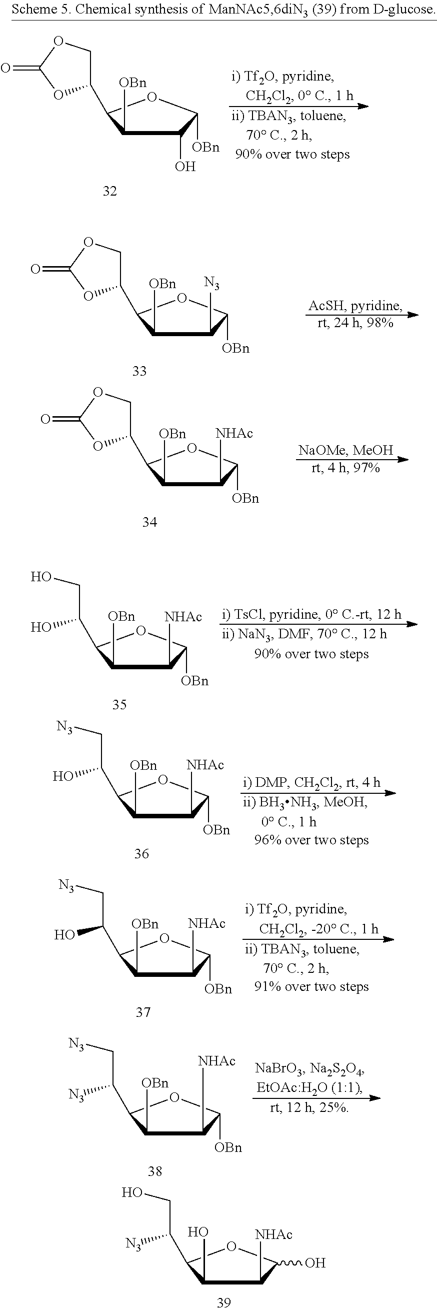

Benzyl 3-O-benzyl-5,6-carbonate-α-D-glucofuranoside (32): 1,2-Isopropylidene ring opening of compound 23 in the presence of benzyl alcohol and Dowex-50 H⁺ ion exchange resin formed α-furanoside product 32 (9.19 g, 50%) as a syrup. ¹H NMR (400 MHz, CDCl₃) δ 7.40-7.26 (m, 8H), 7.25-7.14 (m, 2H), 5.14 (s, 1H), 4.86 (ddd, J=8.6, 6.2, 2.3 Hz, 1H), 4.81 (d, J=7.9 Hz, 1H), 4.79 (s, 1H), 4.73 (dd, J=9.1, 6.1 Hz, 1H), 4.65 (d, J=11.5 Hz, 1H), 4.52 (d, J=11.8 Hz, 1H), 4.41 (d, J=11.6 Hz, 1H), 4.38 (s, 1H), 4.30 (t, J=8.8 Hz, 1H), 4.04 (d, J=5.6 Hz, 1H), 2.50 (s, 1H); ¹³C NMR (100 MHz, CDCl₃) δ 155.6, 137.1, 137.1, 128.7, 128.7, 128.6, 128.6, 128.2, 128.1, 128.1, 128.0, 127.8, 127.8, 108.5, 83.2, 82.0, 77.7, 76.2, 72.1, 70.2, 66.8. ¹H and ¹³C NMR spectra for compound 32 are provided in FIG. 8L.

Benzyl 2-azido-3-O-benzyl-5,6-carbonate-2-deoxy-α-D-mannofuranoside (33): To a solution of compound 32 (6.0 g, 15.53 mmol) in CH₂Cl₂ (60 mL) and pyridine (13 mL), trifluoromethanesulfonic anhydride (7.82 mL, 46.58 mmol) was added dropwise at 0° C. with stirring. After 1 hour, the mixture was diluted with dichloromethane (30 mL) and washed successively with cold 1 M HCl solution, saturated NaHCO₃ and brine, dried over Na₂SO₄, filtrated and concentrated to give crude triflate ester. The crude triflate ester was used directly in the next step without further purification. To the solution of crude triflate ester in dry toluene (40 mL), tetrabutylammonium azide (8.83 g, 31.05 mmol) was added and the reaction mixture was heated at 70° C. with continuous stirring. After 2 hours, the reaction was stopped, solvent was removed and the condensed mixture was diluted with 30 mL of CH₂C02. The organic layer was washed with brine solution, dried over anhydrous Na₂SO₄. After filtration, the solvent was removed under reduced pressure and the product was purified by silica gel chromatography using hexane:ethylacetate (3:1 v/v) to produce compound 33 (5.75 g, 90% over 2 steps) as a colorless syrup. ¹H NMR (600 MHz, CDCl₃) δ 7.42-7.29 (m, 1OH), 5.27 (d, J=5.1 Hz, 1H), 4.92 (d, J=11.5 Hz, 1H), 4.87 (d, J=12.0 Hz, 1H), 4.73 (dd, J=9.2, 5.7 Hz, 1H), 4.66 (d, J=12.0 Hz, 1H), 4.61 (ddd, J=8.7, 5.7, 3.3 Hz, 1H), 4.52 (d, J=11.5 Hz, 1H), 4.44 (dd, J=5.6, 3.3 Hz, 1H), 4.29 (t, J=5.7 Hz, 1H), 4.25 (t, J=8.8 Hz, 1H) 3.34 (t, J=5.4 Hz, 1H); ¹³C NMR (150 MHz, CDCl₃) δ 155.0, 136.7, 136.7, 128.9, 128.9, 128.7, 128.6, 128.6, 128.4, 128.4, 128.1, 128.0, 128.0, 101.8, 81.0, 78.0, 75.5, 75.2, 70.6, 66.6, 61.5. ¹H and ¹³C NMR spectra for compound 33 are provided in FIG. 8M.

Benzyl 3-O-benzyl-5,6-carbonate-α-D-N-acetylmannosamine (34): To a solution of compound 33 (5.60 g, 13.61 mmol) in pyridine (50 mL), thioacetic acid (9.6 mL, 136.12 mmol) was added under argon at room temperature and the reaction mixture was stirred for 24 hours and the product was purified by silica gel chromatography using hexane:ethylacetate (3:1 to 3:2 v/v) as an eluent to produce compound 34 (5.70 g, 98%) as a white solid. ¹H NMR (600 MHz, CDCl₃) δ 7.39-7.29 (m, 8H), 7.24-7.22 (m, 2H), 6.07 (d, J=8.5 Hz, 1H), 5.14 (d, J=5.4 Hz, 1H), 4.81 (d, J=11.8 Hz, 1H), 4.70-4.65 (m, 2H), 4.55-4.54 (m, 2H), 4.52 (d, J=4.8 Hz, 1H), 4.51 (d, J=2.0 Hz, 1H), 4.49-4.48 (m, 1H), 4.32-4.26 (m, 2H), 1.90 (s, 3H); ¹³C NMR (150 MHz, CDCl₃) δ 170.2, 155.0, 137.2, 137.0, 128.9, 128.9, 128.7, 128.7, 128.5, 128.3, 128.1, 128.1, 127.8, 127.8, 100.7, 81.4, 76.8, 75.5, 75.1, 70.6, 66.6, 54.2, 23.1. ¹H and ¹³C NMR spectra for compound 34 are provided in FIG. 8N.

Benzyl 3-O-benzyl-α-D-N-acetylmannosamine (35): To a solution of compound 34 (5.6 g, 13.10 mmol) in dry methanol (50 mL), 30% sodium methoxide in methanol (0.7 mL) was added at room temperature. After 3 hours, the reaction mixture was neutralized with Dowex 50W (H⁺), filtered and concentrated under reduced pressure and purified using flash chromatography hexane:ethyl acetate (1:1 to 1:2, v/v) to produce compound 35 (5.10 g, 97%) as a white solid. ¹H NMR (600 MHz, CDCl₃) δ 7.35-7.29 (m, 1OH), 6.14 (d, J=8.8 Hz, 1H), 5.04 (d, J=5.4 Hz, 1H), 4.78 (d, J=12.0 Hz, 1H), 4.70 (d, J=12.0 Hz, 1H), 4.62 (d, J=11.9 Hz, 1H), 4.53-4.50 (m, 1H), 4.48 (d, J=12.0 Hz, 1H), 4.29 (t, J=5.5 Hz, 1H), 4.06 (dd, J=8.9, 5.0 Hz, 1H), 4.00 (ddd, J=8.7, 5.4, 3.2 Hz, 1H), 3.79 (dd, J=11.4, 3.2 Hz, 1H), 3.68 (dd, J=11.4, 5.4 Hz, 1H), 2.53 (s, 2H), 1.88 (s, 3H); ¹³C NMR (150 MHz, CDCl₃) δ 170.2, 138.0, 137.8, 128.9, 128.9, 128.5, 128.5, 128.2, 127.9, 127.9, 127.9, 127.7, 127.7, 100.2, 80.9, 77.5, 74.8, 70.4, 69.8, 64.2, 54.1, 23.2. ¹H and ¹³C NMR spectra for compound 35 are provided in FIG. 80.

Benzyl 6-azido-3,6-di-O-benzyl-6-deoxy-α-D-N-acetylmannosamine (36): To a stirring solution of pre-dried compound 35 (5 g, 12.45 mmol) in anhydrous pyridine (50 mL) at 0° C., a solution of p-toluenesulfonyl chloride (4.75 g, 24.91 mmol) in pyridine (10 mL) was added and the mixture was stirred at room temperature for 24 h. The reaction was stopped by adding MeOH (10 mL) and the mixture was concentrated. Purification of the residue by column chromatography (EtOAc/MeOH=9:1, by volume) provided the 6-O-tosylated product. The resulting 6-O-Ts-ManNAc (6.44 g 11.59 mmol) was dissolved in anhydrous DMF (50 mL), and sodium azide (7.53 g, 115.90 mmol) was added. The reaction mixture was stirred at 70° C. for 12 hours. The solvent was removed under reduced pressure and purified using flash chromatography with hexane:ethyl acetate (1:1 to 1:2, v/v) as the eluting solvent to produce compound 36 (4.79 g, 90% in two steps). ¹H NMR (400 MHz, CDCl₃) δ 7.36-7.26 (m, 1OH), 6.10 (d, J=8.8 Hz, 1H), 5.05 (d, J=5.3 Hz, 1H), 4.75 (d, J=12.0 Hz, 1H), 4.68-4.61 (m, 2H), 4.55-4.47 (m, 2H), 4.31 (t, J=5.5 Hz, 1H), 4.08-4.01 (m, 2H), 3.48 (dd, J=12.7, 2.7 Hz, 1H), 3.36 (dd, J=12.7, 5.6 Hz, 1H), 2.51 (d, J=4.9 Hz, 1H), 1.89 (s, 3H); ¹³C NMR (100 MHz, CDCl₃) δ 170.1, 137.8, 137.7, 128.9, 128.9, 128.6, 128.6, 128.3, 128.0, 127.9, 127.9, 127.7, 127.7, 100.4, 81.0, 77.2, 74.8, 70.1, 69.7, 54.3, 54.1, 23.2. ¹H and ¹³C NMR spectra for compound 36 are provided in FIG. 8P.

Benzyl 6-azido-3,6-di-O-benzyl-6-deoxy-α-L-N-acetylgulosamine (37): Alcohol 36 (4.50 g, 10.55 mmol) was dissolved in dry CH₂Cl₂ (40 mL), Dess-Martin periodinane (6.71 g, 15.83 mmol) was added and the reaction mixture was stirred at 25° C. for 2 hours. The reaction mixture was diluted with 40 mL of CH₂Cl₂ and washed with sodium thiosulfate and sodium bicarbonate solutions. Combined organic layers was washed with brine solution, dried over anhydrous Na₂SO₄ and concentrated under reduced pressure to get the keto compound which was directly used for the subsequent reaction without any further purification. The keto compound re-dissolved in MeOH (50 mL) and treated with BH₃. NH₃ (31.66 mmol) at 0° C. After 1 hour, the reaction mixture was poured over ice and extracted with EtOAc, washed with brine, and dried over anhydrous Na₂SO₄. The solution was decanted and concentrated under s reduced pressure to obtain a pale yellow residue which upon purification by silica gel column chromatography produced compound 37 (4.32 g, 96% over 2 steps) as a colorless syrup. ¹H NMR (400 MHz, CDCl₃) δ 7.32-7.17 (m, 1OH), 6.03 (d, J=8.7 Hz, 1H), 5.03 (d, J=5.1 Hz, 1H), 4.83 (d, J=11.5 Hz, 1H), 4.52 (s, 2H), 4.48-4.36 (m, 2H), 4.23 (t, J=6.0 Hz, 1H), 4.10 (dd, J=5.9, 2.3 Hz, 1H), 3.87 (t, J=6.1 Hz, 1H), 3.45 (d, J=1.9 Hz, 1H), 3.35 (dd, J=12.4, 6.7 Hz, 1H), 3.25 (dd, J=12.4, 5.5 Hz, 1H), 1.82 (s, 3H); ¹³C NMR (100 MHz, CDCl₃) δ 170.1, 137.3, 137.0, 128.9, 128.9, 128.6, 128.6, 128.4, 128.4, 128.4, 128.1, 127.9, 127.9, 99.8, 80.6, 78.4, 75.1, 69.9, 69.8, 54.1, 53.5, 23.2. ¹H and ¹³C NMR spectra for compound 37 are provided in FIG. 8Q.

Benzyl 5,6-di-azido-3,6-di-O-benzyl-5,6-di-deoxy-α-D-N-acetylmannosamine (38): To a solution of compound 37 (3.0 g, 7.03 mmol) in CH₂Cl₂ (30 mL) in pyridine (5 mL), trifluoromethanesulfonic anhydride (3.54 mL, 21.10 mmol) was added drop-wisely at −20° C. with stirring. After 1 hour, the mixture was diluted with dichloromethane (30 mL) and washed successively with cold 1 M HCl solution, saturated NaHCO₃ and brine, dried over Na₂SO₄, filtrated and concentrated to produce the crude triflate ester. The crude triflate ester was used directly in the next step without further purification. To the solution of crude triflate ester in dry toluene (40 mL), tetrabutylammonium azide (6.01 g, 21.11 mmol) was added and the reaction mixture was heated at 70° C. with continuous stirring. After 2 hours, the reaction was stopped, solvent was removed, and the condensed mixture was diluted with 30 mL of CH₂Cl₂. The organic layer was washed with brine solution, dried over anhydrous Na₂SO₄. After filtration, the solvent was removed under reduced pressure and the product was purified by silica gel chromatography using hexane:ethylacetate (3:1 to 2:1, by volume) to produce compound 38 (2.89 g, 91% over 2 steps) as a white solid. ¹H NMR (600 MHz, CDCl₃) δ 7.36-7.29 (m, 1OH), 6.01 (d, J=8.8 Hz, 1H), 5.06 (d, J=5.4 Hz, 1H), 4.82 (d, J=11.8 Hz, 1H), 4.72 (d, J=12.1 Hz, 1H), 4.55 (d, J=5.2 Hz, 1H), 4.53-4.50 (m, 2H), 4.27 (t, J=5.2 Hz, 1H), 3.99 (dd, J=9.7, 4.9 Hz, 1H), 3.88 (ddd, J=9.6, 6.9, 2.7 Hz, 1H), 3.62 (dd, J=12.9, 2.7 Hz, 1H), 3.40 (dd, J=12.9, 6.9 Hz, 1H), 1.79 (s, 3H); ¹³C NMR (150 MHz, CDCl₃) δ 170.0, 138.0, 137.8, 128.7, 128.7, 128.6, 128.6, 128.0, 127.9, 127.7, 127.7, 127.4, 127.4, 100.8, 79.9, 77.4, 75.0, 70.4, 60.7, 54.2, 52.9, 23.0. ¹H and ¹³C NMR spectra for compound 38 are provided in FIG. 8R.

ManNAc5,6diN₃ (39): To a solution of compound 38 (1.0 g, 2.21 mmol) in EtOAc (10 mL), a solution of NaBrO₃ (2.01 g, 13.29 mmol) in water (5 mL) was added. A solution of Na₂S204 (1.93 g, 11.07 mmol) in water (5 mL) was then added over 25 minutes and the mixture was vigorously stirred for 12 hours at ambient temperature. After that the mixture was quenched with 10% sodium thiosulphate (2 mL) and neutralized with 1 M NaOH solution. The water layer was separated from the organic layer and filtered to remove the white precipitate formed during the neutralization process. The filtrate was evaporated and the crude product was purified using a Bio-Gel P-2 Gel (Bio-Rad) column and a C18 reverse phase column to produce ManNAc5,6diN₃ (39) as a α/β mixture (150 mg, 25%) as white solid. HRMS (ESI-Orbitrap) m/z: [M+Cl]⁻ Calcd for C₈H₁₃N₇O₄Cl 306.0718; found 306.0947. HRMS spectrum of compound 39 is provided in FIG. 8T.

### 3. Chemical synthesis of ManNAc5,6diN₃ (39) from ManNAc5N₃ β1)

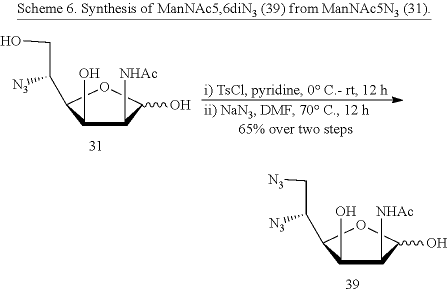

To a stirring solution of pre-dried compound 31 (20 mg, 81.23 μmol) in anhydrous pyridine (1 mL) at 0° C., a solution of p-toluenesulfonyl chloride (24 mg, 121.84 μmol) in pyridine (0.5 mL) was added, and the mixture was stirred at room temperature for 12 hours. The reaction was stopped by adding MeOH (0.5 mL) and the mixture was concentrated. Purification of the residue by column chromatography (EtOAc:MeOH=9:1, by volume) provided the 6-O-tosylated product which was dissolved in anhydrous DMF (3 mL), and sodium azide (35 mg, 550 μmol) was added. The reaction mixture was stirred at 70° C. for 12 hours. The formation of ManNAc5,6diN₃ (39) was confirmed by TLC and a 65% overall yield was estimated based on TLC results.

### 4. Chemoenzymatic synthesis of Neu5Ac8NAc (46)

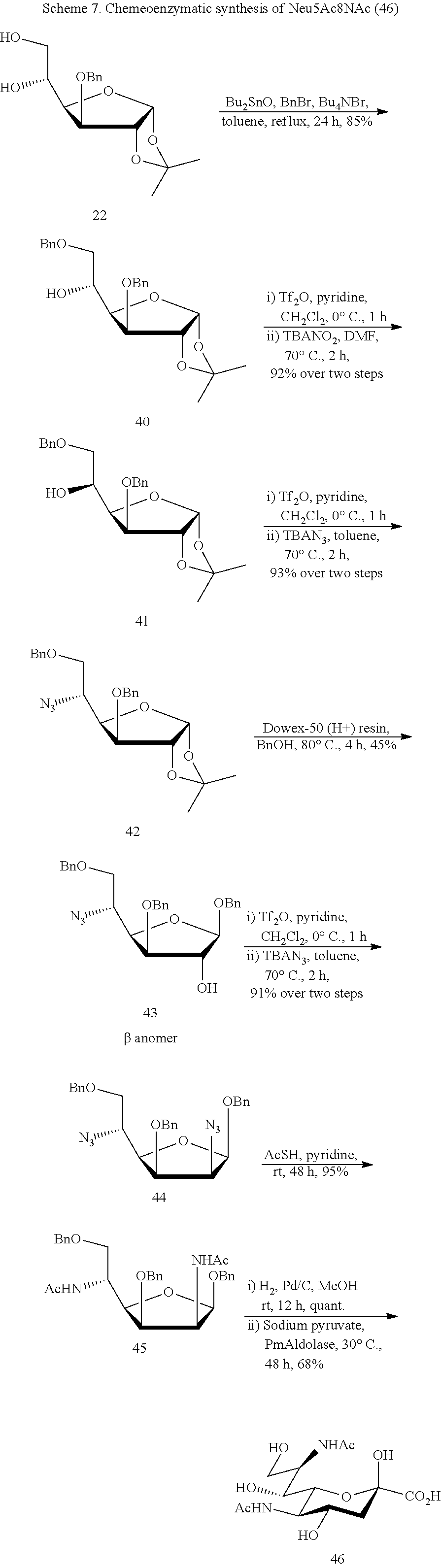

3,6-Di-O-benzyl-1,2-O-isopropylidene-α-D-glucofuranose (40): To the suspended mixture of compound 22 (5.0 g, 16.11 mmol) and dibutyltin oxide (6.14 g, 24.17 mmol) in dry toluene (80 mL) were heated under reflux for 3 hours. Approximately 40 mL solvent was azeotropicly removed using a Dean-stark apparatus under nitrogen atmosphere. After that tetrabutylammonium bromide (260 mg, 0.806 mmol) and benzyl bromide (2.5 mL, 20.94 mmol) were added into the reaction mixture and heated under reflux for 24 hours. The solvent was evaporated; the residue was diluted with ethyl acetate, washed successively with 10% aqueous KF solution and brine, dried over Na₂SO₄, and concentrated. The residue was purified by flash chromatography with hexane:ethylacetate (8:1 to 6:1, v/v) to give compound 40 (5.48 g, 85%) as a syrup. ¹H NMR (400 MHz, CDCl₃) δ 7.29-7.19 (m, 1OH), 5.84 (d, J=3.7 Hz, 1H), 4.59 (d, J=11.4 Hz, 1H), 4.54-4.43 (m, 4H), 4.12-4.05 (m, 2H), 4.03 (d, J=2.3 Hz, 1H), 3.66 (dd, J=9.5, 2.4 Hz, 1H), 3.53 (dd, J=9.8, 4.8 Hz, 1H), 2.62 (s, 1H), 1.41 (s, 3H), 1.24 (s, 3H); ¹³C NMR (100 MHz, CDCl₃) δ 138.1, 137.5, 128.7, 128.7, 128.5, 128.5, 128.1, 128.0, 128.0, 127.9, 127.9, 127.9, 111.9, 105.3, 82.4, 82.2, 79.9, 73.6, 72.5, 72.2, 68.2, 26.9, 26.4. ¹H and ¹³C NMR spectra for compound 40 are provided in FIG. 8U. 3,6-Di-O-benzyl-1,2-O-isopropylidene-α-L-idofuranose (41): To a solution of compound 40 (5.4 g, 13.48 mmol) in CH₂Cl₂ (50 mL) in pyridine (10 mL), trifluoromethanesulfonic anhydride (6.8 mL, 40.45 mmol) was added drop-wisely at 0° C. with stirring. After 1 hour, the mixture was diluted with dichloromethane (50 mL) and washed successively with cold 1 M HCl solution, saturated NaHCO₃ and brine, dried over Na₂SO₄, filtrated and concentrated to give crude triflate ester which was used directly in the next step without further purification. The crude triflate ester was re-dissolved in dry DMF (60 mL) and tetrabutylammonium nitrite (11.67 g, 40.45 mmol) was added. The reaction mixture was heated at 70° C. with continuous stirring. After 2 h, the reaction was stopped, solvent was removed and the condensed mixture was diluted with 75 mL of CH₂C02. The organic layer was washed with brine solution, dried over anhydrous Na₂SO₄. After filtration, the solvent was removed under reduced pressure and the product was purified by silica gel chromatography using hexane:ethylacetate (8:1 to 6:1, v/v) to produce L-idose compound 41 (4.97 g, 92% over 2 steps) as colorless syrup. ¹H NMR (400 MHz, CDCl₃) δ 7.30-7.16 (m, 1OH), 5.92 (d, J=3.8 Hz, 1H), 4.58 (d, J=11.9 Hz, 1H), 4.55 (d, J=4.4 Hz, 1H), 4.47 (d, J=12.0 Hz, 1H), 4.40 (d, J=12.1 Hz, 1H), 4.30 (d, J=11.7 Hz, 1H), 4.20 (t, J=4.4 Hz, 1H), 4.09 (q, J=5.3 Hz, 1H), 3.87 (d, J=3.5 Hz, 1H), 3.51-3.39 (m, 2H), 2.91 (s, 1H), 1.42 (s, 3H), 1.26 (s, 3H); ¹³C NMR (100 MHz, CDCl₃) δ 138.2, 136.9, 128.8, 128.8, 128.5, 128.5, 128.4, 128.0, 128.0, 128.0, 128.0, 127.8, 112.0, 105.0, 83.1, 82.5, 79.9, 73.6, 72.0, 70.9, 69.6, 27.0, 26.5. ¹H and ¹³C NMR spectra for compound 41 are provided in FIG. 8V.

5-Azido-3,6-Di-O-benzyl-5-deoxy-1,2-O-isopropylidene-α-D-glucofuranose (42): To a solution of compound 41 (4.9 g, 12.24 mmol) in CH₂Cl₂ (50 mL) in pyridine (10 mL), trifluoromethanesulfonic anhydride (6.16 mL, 36.71 mmol) was added drop-wisely at 0° C. with stirring. After 1 hour, the mixture was diluted with dichloromethane (50 mL) and washed successively with cold 1 M HCl solution, saturated NaHCO₃ and brine, dried over Na₂SO₄, filtrated and concentrated to produce the crude triflate ester. The crude triflate ester was used directly in the next step without further purification. To the solution of crude triflate ester in dry toluene (70 mL), tetrabutylammonium azide (10.45 g, 36.73 mmol) was added and the reaction mixture was heated at 70° C. with continuous stirring. After 2 hours, the reaction was stopped, solvent was removed and the condensed mixture was diluted with 50 mL of CH₂C₂. The organic layer was washed with brine solution, dried over anhydrous Na₂SO₄. After filtration, the solvent was removed under reduced pressure and the product was purified by silica gel chromatography using hexane:ethylacetate (6:1 to 3:1 v/v) to produce compound 42 (4.84 g, 93% over 2 steps) as a yellow syrup. ¹H NMR (400 MHz, CDCl₃) δ 7.33-7.16 (m, 1OH), 5.80 (d, J=3.7 Hz, 1H), 4.60 (d, J=11.4 Hz, 1H), 4.53-4.51 (m, 4H), 4.00-3.91 (m, 3H), 3.83 (dt, J=10.2, 1.7 Hz, 1H), 3.56 (dd, J=10.1, 7.2 Hz, 1H), 1.39 (s, 3H), 1.23 (s, 3H); ¹³C NMR (100 MHz, CDCl₃) δ 138.1, 137.3, 128.6, 128.6, 128.5, 128.5, 128.2, 128.1, 128.1, 127.8, 127.7, 127.7, 112.1, 105.4, 81.8, 81.7, 78.7, 73.5, 72.3, 71.2, 59.2, 26.9, 26.4. ¹H and ¹³C NMR spectra for compound 42 are provided in FIG. 8W.

Benzyl 5-azido-3,6-di-O-benzyl-5-deoxy-β-D-glucofuranoside (43): A solution of compound 42 (4.50 g, 10.58 mmol) in benzyl alcohol (50 mL) was stirred at 80° C. for 4 hours in the presence of Dowex-50 H⁺ ion exchange resin (5 g). The filtered solution was purified on silica gel column and washed first with hexane:ethyl acetate (19:1, v/v) to remove most of the benzyl alcohol, then washed with hexane:ethyl acetate (9:1 to 4:1, v/v) to give P-anomer 43 (2.26 g, 45%) as colorless syrup. The a-anomer was co-eluted with benzyl alcohol and could not able to separate. ¹H NMR (400 MHz, CDCl₃) δ 7.32-7.16 (m, 15H), 4.89 (s, 1H), 4.62 (d, J=9.1 Hz, 1H), 4.59 (d, J=9.1 Hz, 1H), 4.54 (d, J=11.5 Hz, 1H), 4.49 (s, 2H), 4.36 (d, J=12.0 Hz, 1H), 4.24 (d, J=3.3 Hz, 1H), 4.14 (dd, J=9.8, 5.0 Hz, 1H), 3.97 (ddd, J=9.6, 6.9, 2.3 Hz, 1H), 3.89 (d, J=5.0 Hz, 1H), 3.77 (dd, J=10.2, 2.5 Hz, 1H), 3.55 (dd, J=10.2, 7.2 Hz, 1H), 1.80 (s, 1H); ¹³C NMR (100 MHz, CDCl₃) δ 138.1, 137.7, 137.7, 128.5, 128.5, 128.5, 128.5, 128.5, 128.5, 128.1, 128.1, 127.9, 127.9, 127.9, 127.8, 127.7, 127.7, 127.7, 108.1, 82.8, 80.0, 78.2, 73.5, 72.2, 71.0, 69.8, 60.4. ¹H and ¹³C NMR spectra for compound 43 are provided in FIG. 8X.

Benzyl 2,5-di-azido-3,6-di-O-benzyl-2,5-di-deoxy-β-D-glucofuranoside (44): To a solution of compound 43 (2.2 g, 4.63 mmol) in CH₂Cl₂ (20 mL) in pyridine (4 mL), trifluoromethanesulfonic anhydride (2.33 mL, 13.88 mmol) was added drop-wisely at 0° C. with stirring. After 1 hour, the mixture was diluted with dichloromethane (20 mL) and washed successively with cold 1 M HCl solution, saturated NaHCO₃ and brine, dried over Na₂SO₄, filtrated and concentrated to give crude triflate ester. The crude triflate ester was used directly in the next step without further purification. To the solution of crude triflate ester in dry toluene (70 mL), tetrabutylammonium azide (3.95 g, 13.87 mmol) was added and the reaction mixture was heated at 70° C. with continuous stirring. After 2 hours, the reaction was stopped, solvent was removed and the condensed mixture was diluted with 20 mL of CH₂C₂. The organic layer was washed with brine solution, dried over anhydrous Na₂SO₄. After filtration, the solvent was removed under reduced pressure and the product was purified by silica gel chromatography using hexane:ethylacetate (10:1 to 8:1 v/v) to produce compound 44 (2.11 g, 91% over 2 steps) as a yellow syrup. ¹H NMR (400 MHz, CDCl₃) δ 7.42-7.28 (m, 15H), 5.01 (s, 1H), 4.77-4.69 (m, 2H), 4.65 (d, J=11.5 Hz, 1H), 4.61 (s, 2H), 4.48 (d, J=12.0 Hz, 1H), 4.34 (d, J=4.2 Hz, 1H), 4.26 (dd, J=9.7, 5.0 Hz, 1H), 4.09 (ddd, J=9.7, 7.1, 2.5 Hz, 1H), 4.00 (dd, J=5.0, 1.3 Hz, 1H), 3.89 (dd, J=10.2, 2.5 Hz, 1H), 3.67 (dd, J=10.2, 7.1 Hz, 1H); ¹³C NMR (100 MHz, CDCl₃) δ 138.1, 137.7, 137.7, 128.5, 128.5, 128.5, 128.5, 128.5, 128.5, 128.1, 128.1, 127.9, 127.9, 127.8, 127.8, 127.7, 127.7, 127.7, 108.1, 82.8, 80.0, 78.2, 73.5, 72.2, 71.0, 69.8, 60.3. ¹H and ¹³C NMR spectra for compound 44 are provided in FIG. 8Y.

Benzyl 5-N-acetyl-3,6-di-O-benzyl-5-deoxy-β-D-N-acetylmannosamine (45): To a solution of compound 44 (600 mg, 1.20 mmol) in pyridine (10 mL), thioacetic acid (2.53 mL, 35.96 mmol) was added under argon at room temperature and the reaction mixture was stirred for 48 hours and the product was purified by silica gel chromatography using hexane:ethylacetate (4:1 to 2:1 v/v) as an eluent to produce compound 45 (606 mg, 95%) as a white solid. ¹H NMR (400 MHz, CDCl₃) δ 7.60-7.03 (m, 15H), 6.30 (d, J=8.0 Hz, 1H), 6.23 (d, J=9.2 Hz, 1H), 5.00 (d, J=2.2 Hz, 1H), 4.71 (d, J=11.9 Hz, 1H), 4.63-4.56 (m, 2H), 4.56-4.45 (m, 4H), 4.38-4.32 (m, 3H), 3.75 (dd, J=9.6, 3.5 Hz, 1H), 3.63 (dd, J=9.6, 4.5 Hz, 1H), 1.91 (s, 3H), 1.87 (s, 3H); ¹³C NMR (100 MHz, CDCl₃) δ 170.1, 170.0, 138.0, 137.6, 137.2, 128.8, 128.8, 128.6, 128.6, 128.5, 128.5, 128.5, 128.5, 128.5, 128.1, 128.1, 128.0, 128.0, 127.9, 127.8, 105.8, 79.4, 77.4, 74.6, 73.6, 69.7, 69.7, 56.8, 48.9, 23.6, 23.3. ¹H and ¹³C NMR spectra for compound 45 are provided in FIG. 8Z.

Neu5Ac8NAc (46): To a solution of compound 45 (500 mg, 0.938 mmol) in MeOH (10 mL), Pd/C acid (50 mg) was added under hydrogen atmosphere at room temperature and the mixture was stirred for 12 hours. After completion of the reaction, Pd/C was removed using a celite bed and the filtrate was concentrated under a reduced pressure. The product (100 mg, 0.381 mmol)) was used for the formation of Neu5Ac8NAc by incubating with sodium pyruvate (210 mg, 1.91 mmol) and PmAldolase (12 mg) in water at 30° C. for 48 h with agitation at 180 rpm. The product formation was monitored by TLC with EtOAc:MeOH:H₂O=5:2:1 (by volume) as the developing solvent and by HRMS. After the reaction was completed, the reaction mixture was incubated in a boiling water bath for 5 minutes to denature the enzymes, then centrifuge at 8000 rpm for 30 minutes at 4° C. The supernatant was concentrated and purified by a C18 cartridge (51 g, 50 μm, 120 A, Yamazen) on a CombiFlash system to obtain Neu5Ac8NAc (46) as a white powder (91 mg, 68%). ¹H NMR (400 MHz, D₂O) δ 4.11 (ddd, J=8.2, 6.7, 4.1 Hz, 1H), 3.99 (ddd, J=11.4, 9.9, 4.7 Hz, 1H), 3.89 (t, J=10.1 Hz, 1H), 3.80-3.78 (m, 1H), 3.77-3.75 (m, 1H), 3.73 (dd, J=8.3, 1.0 Hz, 1H), 3.68 (dd, J=11.6, 6.8 Hz, 1H), 2.22 (dd, J=8.1, 4.8 Hz, 1H), 2.06 (s, 3H), 2.03 (s, 3H), 1.82 (dd, J=12.9, 11.5 Hz, 1H); ¹³C NMR (100 MHz, CDCl₃) δ 176.4, 174.7, 174.2, 96.3, 70.7, 67.1, 67.1, 60.9, 52.8, 52.4, 39.4, 22.2, 22.1; HRMS (ESI-Orbitrap) m/z: [M−H]⁻ Calcd for C₁₃H₂₂N₂O₉ 349.1247; found 349.1254. ¹H and ¹³C NMR spectra for compound 46 are provided in FIG. 8AA. HRMS spectrum of compound 46 is provided in FIG. 8BB.

SEQUENCES
SEQ ID NO: 1:
EKQNIAVILARQNSKGLPLKNLRKMNGISLLGHTINAAISSKCFDRIIVS
TDGGLIAEEAKNFGVEVVLRPAELASDTASSISGVIHALETIGSNSGTVT
LLQPTSPLRTGAHIREAFSLFDEKIKGSVVSACPMEHHPLKTLLQINNGE
YAPMRHLSDLEQPRQQLPQAFRPNGAIYINDTASLIANNCFFIAPTKLYI
MSHQDSIDIDTELDLQQAENILHHKES
SEQ ID NO: 2:
DKFAEHEIPKAVIVAGNGESLSQIDYRLLPKNYDVFRCNQFYFEERYFLG
NKIKAVFFTPGVFLEQYYTLYHLKRNNEYFVDNVILSSFNHPTVDLEKSQ
KIQALFIDVINGYEKYLSKLTAFDVYLRYKELYENQRITSGVYMCAVAIA
MGYTDIYLTGIDFYQASEENYAFDNKKPNIIRLLPDFRKEKTLFSYHSKD
IDLEALSFLQQHYHVNFYSISPMSPLSKHFPIPTVEDDCETTFVAPLKEN
YINDILLVDKLAAALE
SEQ ID NO: 3:
KKVIIAGNGPSLKEIDYSRLPNDFDVFRCNQFYFEDKYYLGKKCKAVFYN
PSLFFEQYYTLKHLIQNEYETELIMCSNYNQAHLENENFVKTFYDYFPDA
HLGYDFFKQLKDFNAYFKFHEIYFNQRITSGVYMCAVAIALGYKEIYLSG
IDFYQNGSSYAFDTKQKNLLKLAPNFKNDNSHYIGHSKNTDIKALEFLEK
TYKIKLYCLCPNSLLANFIELAPNLMSNFIIQEKNNYTKDILIPSSEAYG
KFSKNIN
SEQ ID NO: 4:
ATGAAAATCGAAGAAGGTAAACTGGTAATCTGGATTAACGGCGATAAAGG
CTATAACGGTCTCGCTGAAGTCGGTAAGAAATTCGAGAAAGATACCGGAA
TTAAAGTCACCGTTGAGCATCCGGATAAACTGGAAGAGAAATTCCCACAG
GTTGCGGCAACTGGCGATGGCCCTGACATTATCTTCTGGGCACACGACCG
CTTTGGTGGCTACGCTCAATCTGGCCTGTTGGCTGAAATCACCCCGGACA
AAGCGTTCCAGGACAAGCTGTATCCGTTTACCTGGGATGCCGTACGTTAC
AACGGCAAGCTGATTGCTTACCCGATCGCTGTTGAAGCGTTATCGCTGAT
TTATAACAAAGATCTGCTGCCGAACCCGCCAAAAACCTGGGAAGAGATCC
CGGCGCTGGATAAAGAACTGAAAGCGAAAGGTAAGAGCGCGCTGATGTTC
AACCTGCAAGAACCGTACTTCACCTGGCCGCTGATTGCTGCTGACGGGGG
TTATGCGTTCAAGTATGAAAACGGCAAGTACGACATTAAAGACGTGGGCG
TGGATAACGCTGGCGCGAAAGCGGGTCTGACCTTCCTGGTTGACCTGATT
AAAAACAAACACATGAATGCAGACACCGATTACTCCATCGCAGAAGCTGC
CTTTAATAAAGGCGAAACAGCGATGACCATCAACGGCCCGTGGGCATGGT
CCAACATCGACACCAGCAAAGTGAATTATGGTGTAACGGTACTGCCGACC
TTCAAGGGTCAACCATCCAAACCGTTCGTTGGCGTGCTGAGCGCAGGTAT
TAACGCCGCCAGTCCGAACAAAGAGCTGGCAAAAGAGTTCCTCGAAAACT
ATCTGCTGACTGATGAAGGTCTGGAAGCGGTTAATAAAGACAAACCGCTG
GGTGCCGTAGCGCTGAAGTCTTACGAGGAAGAGTTGGCGAAAGATCCACG
TATTGCCGCCACTATGGAAAACGCCCAGAAAGGTGAAATCATGCCGAACA
TCCCGCAGATGTCCGCTTTCTGGTATGCCGTGCGTACTGCGGTGATCAAC
GCCGCCAGCGGTCGTCAGACTGTCGATGAAGCCCTGAAAGACGCGCAGAC
TAATTCGAGCTCGAACAACAACAACAATAACAATAACAACAACCTCGGGA
TCGAGGGAAGGATTTCAGAATTCATGACACGCACCAGAATGGAGAACGAG
TTGATTGTCTCAAAGAACATGCAAAACATCATTATCGCAGGCAATGGCCC
CAGCCTCAAGAATATTAATTACAAGCGTCTCCCGCGGGAATACGACGTTT
TCCGTTGTAACCAATTTTACTTCGAGGACAAGTATTACTTAGGGAAGAAG
ATAAAGGCAGTTTTCTTTAACCCTGGCGTATTCTTACAACAATACCACAC
CGCTAAGCAACTTATACTCAAGAATGAATACGAGATCAAGAACATTTTCT
GTTCCACTTTCAATCTCCCGTTTATTGAGAGCAACGATTTCCTGCACCAA
TTTTACAACTTCTTCCCCGATGCAAAACTGGGCTACGAGGTTATCGAGAA
CCTGAAGGAATTTTACGCCTACATCAAGTATAATGAGATATACTTTAACA
AGCGTATCACATCCGGGGTATATATGTGTGCCATCGCCATAGCCCTGGGT
TACAAGACCATTTACTTGTGCGGAATTGACTTCTACGAGGGCGATGTGAT
CTATCCATTCGAGGCTATGTCAACTAATATTAAGACCATCTTCCCGGGCA
TTAAAGACTTTAAGCCGAGCAATTGTCATAGTAAAGAGTACGACATCGAA
GCATTAAAACTTCTGAAATCGATATACAAGGTGAATATATACGCGCTCTG
TGACGACTCCATATTAGCAAACCATTTCCCTTTGAGCATCAACATCAATA
ACAATTTCACATTGGAGAACAAGCACAACAATAGTATCAACGACATCCTG
TTAACTGACAACACACCGGGTGTTTCATTTTACAAGAATCAACTGAAAGC
AGATAACAAGATAATGCTGAACTTTTATCATCATCATCATCATCATTAA
SEQ ID NO: 5:
MKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRY
NGKLIAYPIAVEALSLIYNKDLLPNPPKTWEEIPALDKELKAKGKSALME
NLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPT
FKGQPSKPFVGVLSAGINAASPNKELAKEFLENYLLTDEGLEAVNKDKPL
GAVALKSYEEELAKDPRIAATMENAQKGEIMPNIPQMSAFWYAVRTAVIN
AASGRQTVDEALKDAQTNSSSNNNNNNNNNNLGIEGRISEFMTRTRMENE
LIVSKNMQNIIIAGNGPSLKNINYKRLPREYDVFRCNQFYFEDKYYLGKK
IKAVFFNPGVFLQQYHTAKQLILKNEYEIKNIFCSTFNLPFIESNDFLHQ
FYNFFPDAKLGYEVIENLKEFYAYIKYNEIYFNKRITSGVYMCAIAIALG
YKTIYLCGIDFYEGDVIYPFEAMSTNIKTIFPGIKDFKPSNCHSKEYDIE
ALKLLKSIYKVNIYALCDDSILANHFPLSININNNFTLENKHNNSINDIL
LTDNTPGVSFYKNQLKADNKIMLNFYHHHHHH
SEQ ID NO: 6
HHHHHH
SEQ ID NO: 7
WSHPQFEK
SEQ ID NO: 8
DYKDDDDK

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A compound of Formula I: 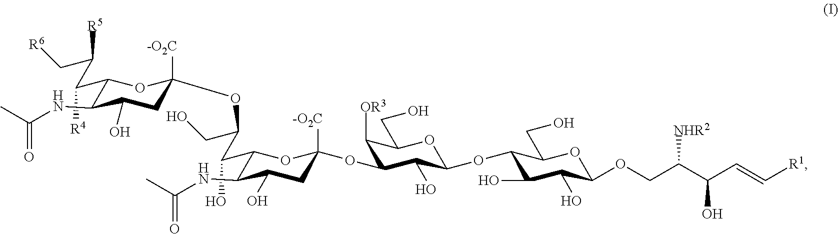
or a salt thereof, wherein:
R¹ is an alkyl chain;
R² is H, —C(O)alkyl, alkyl, —C(O)alkenyl, alkenyl, —C(O)alkynyl, or alkynyl;
R³ is H, a monosaccharide, or an oligosaccharide; and
R⁴, R⁵, and R⁶ are each independently selected from OH, NHAc, NH₂, and N₃, wherein at least one of R⁴, R⁵, and R⁶ is NHAc, NH₂, or N₃.

2. The compound of claim 1, wherein R¹ is C₁-C₂₅ alkyl.

3. The compound of claim 1, wherein R¹ is —C₁₃H₂₇ or —C₁₅H₃₁.

4. The compound of claim 1, wherein R² is —C(O)C₁-C₂₅ alkyl, —C₁-C₂₅ alkyl, —C(O)C₂-C₂₅ alkenyl, —C₂-C₂₅ alkenyl, —C(O)C₂-C₂₅ alkynyl, or —C₂-C₂₅ alkynyl.

5. The compound of claim 1, wherein R² is —C(O)C₁₅H₃₁, —C(O)C₁₇H₃₅, or —C(O)C₁₉H₃₉.

6. The compound of claim 1, wherein R³ comprises a substituted or unsubstituted GalNAc.

7. The compound of claim 1, wherein R³ is GalNAc.

8. The compound of claim 1, wherein R³ is Gal-GalNAc.

9. The compound of claim 1, wherein R³ is Neu5Ac-Gal-GalNAc.

10. The compound of claim 1, wherein R³ is Neu5Ac-Neu5Ac-Gal-GalNAc.

11. The compound of claim 1, wherein R³ is H.

12. The compound of of claim 1, wherein the compound is selected from the group consisting of:

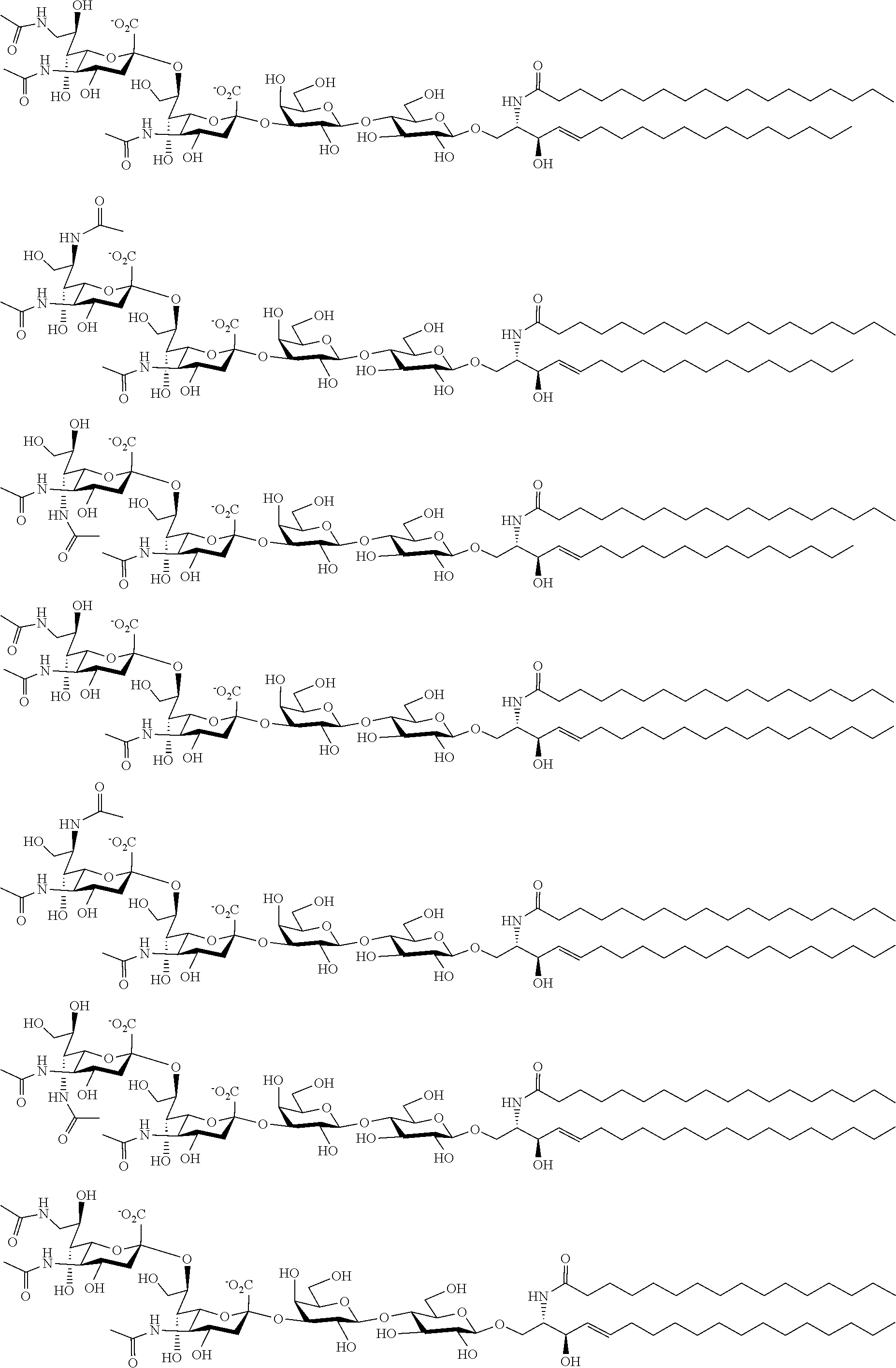

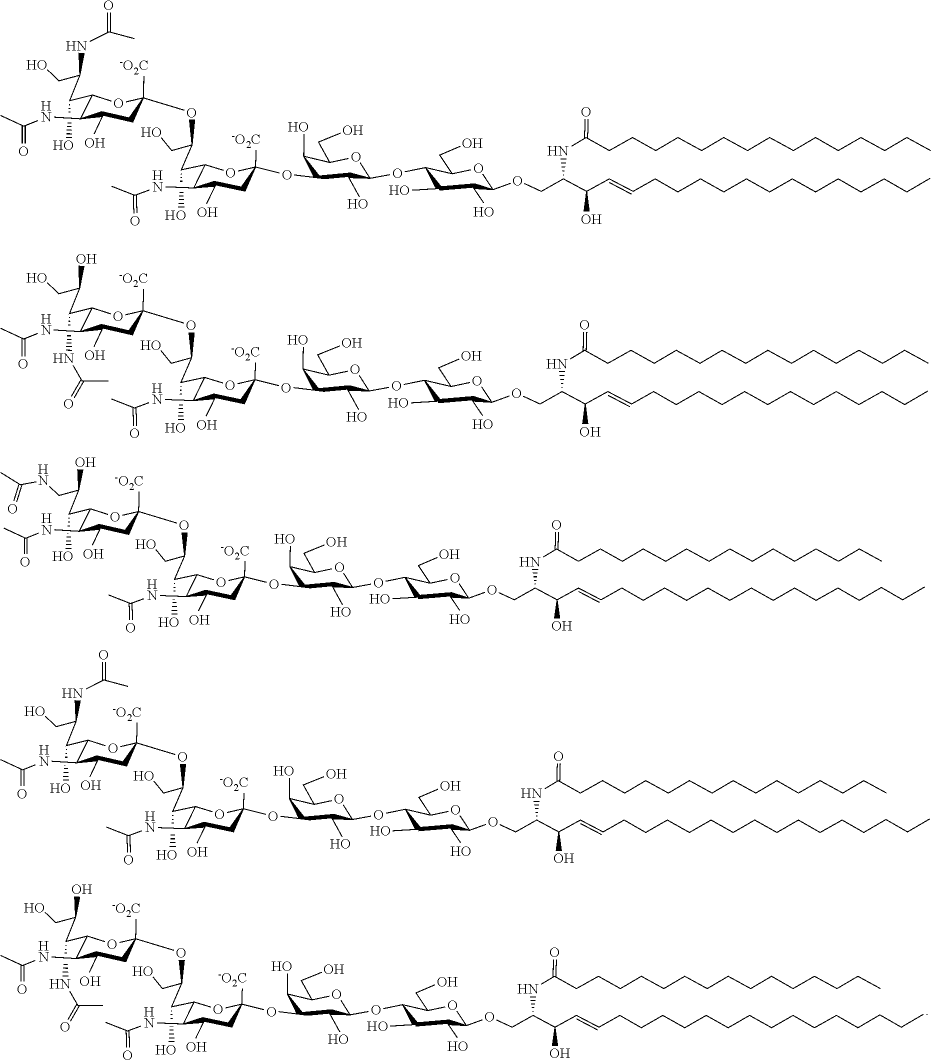

13. The compound of claim 1, wherein the compound is selected from the group consisting of:

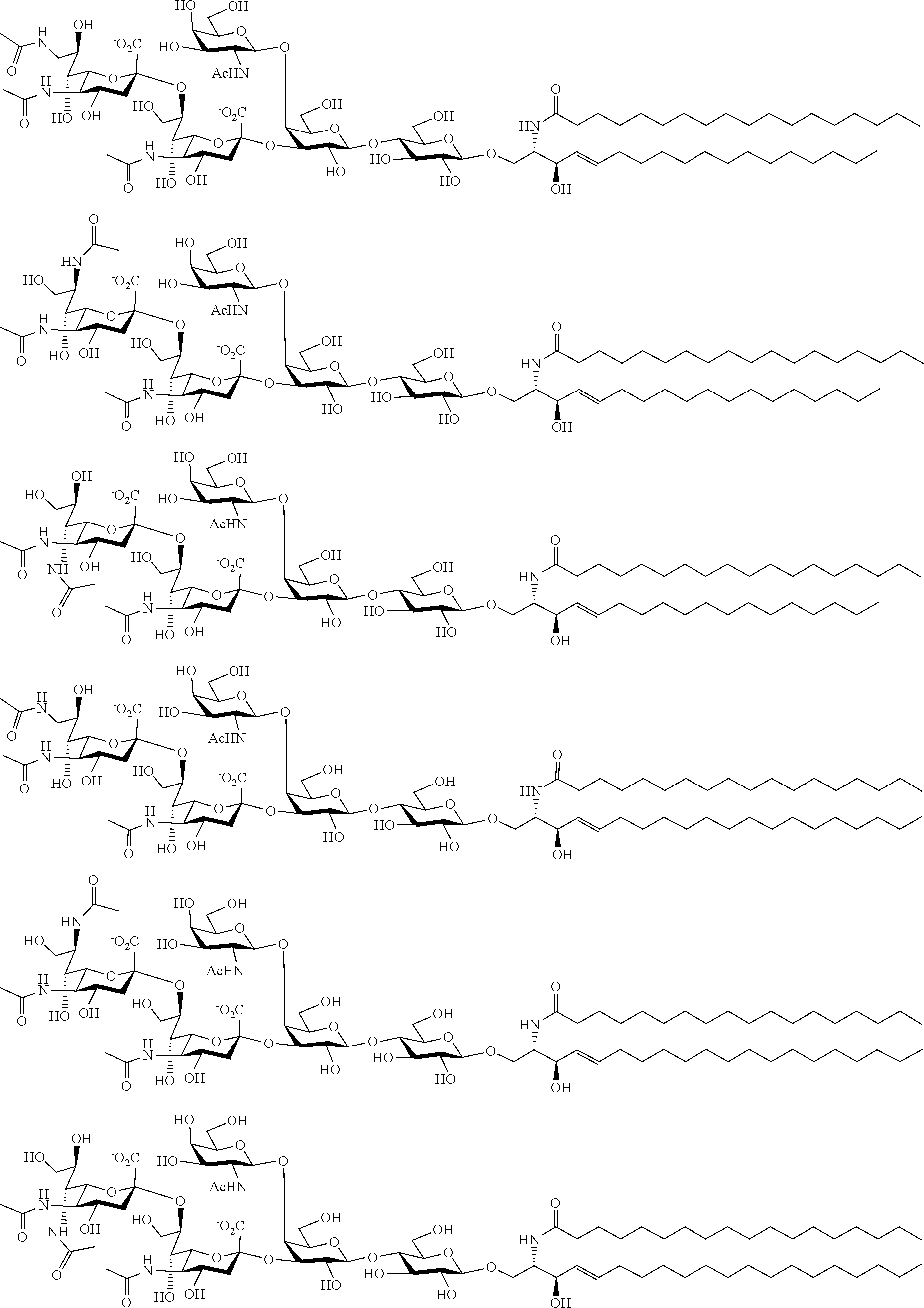

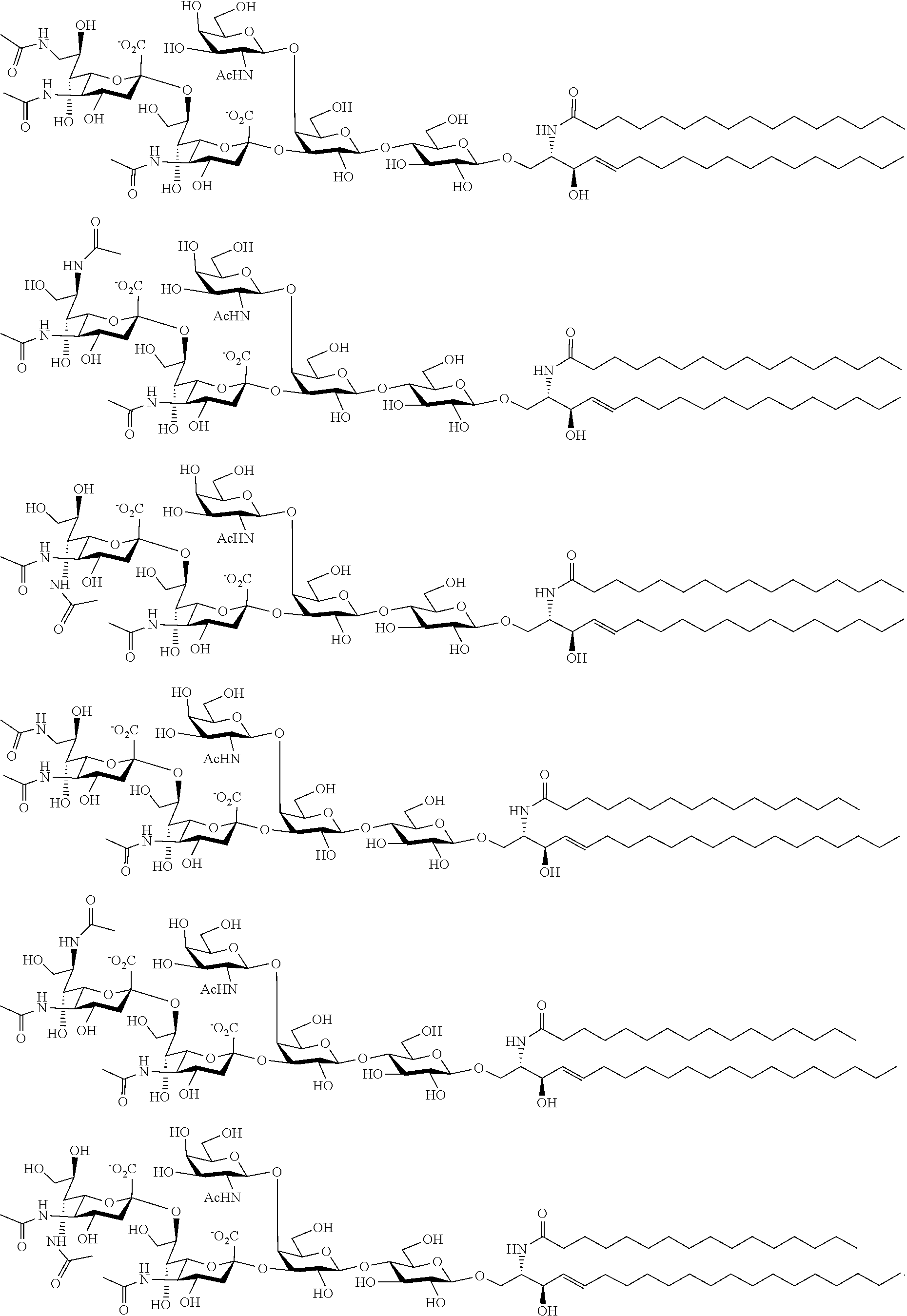

14. The compound of claim 1, wherein the compound is selected from the group consisting of:

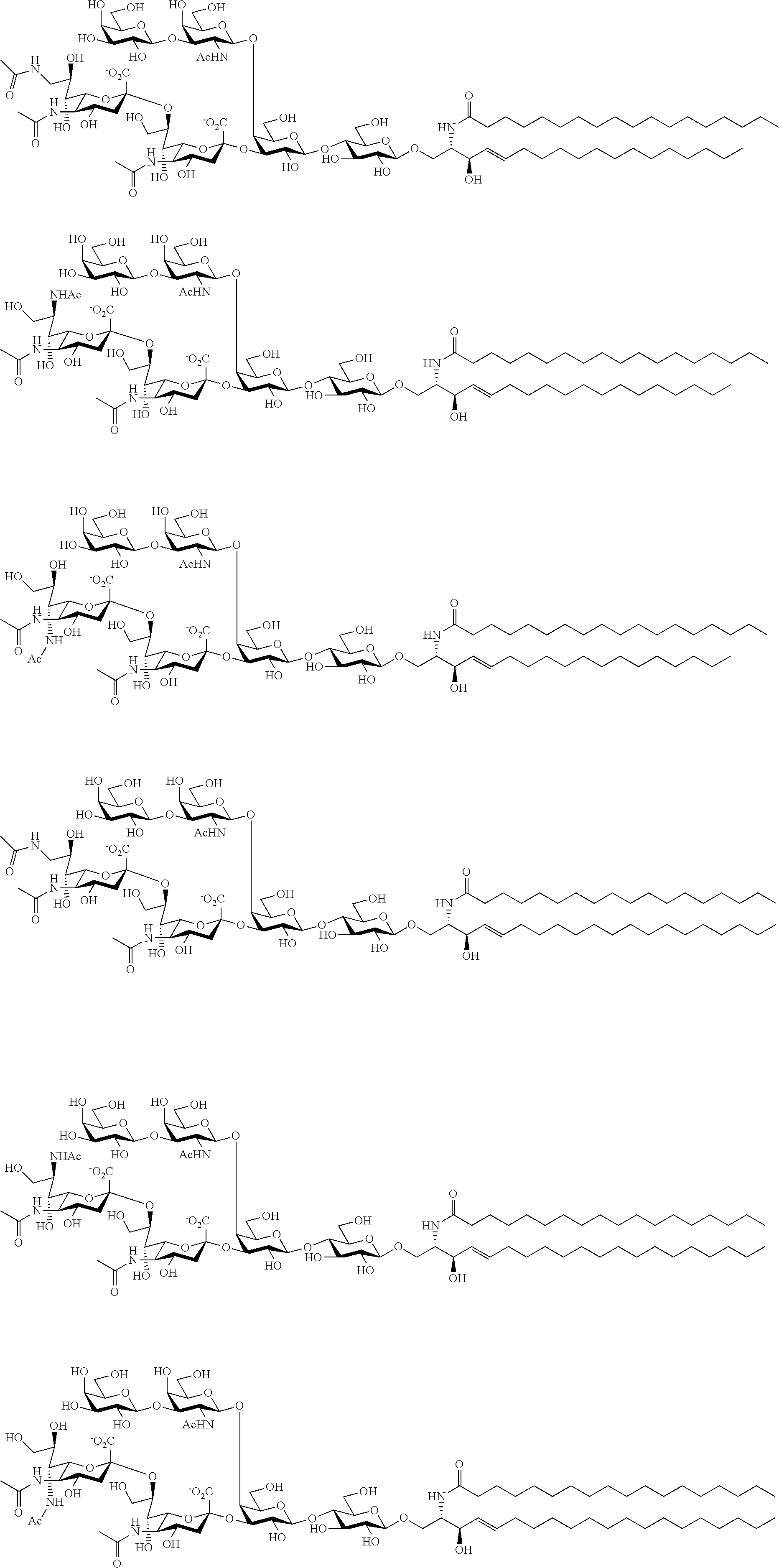

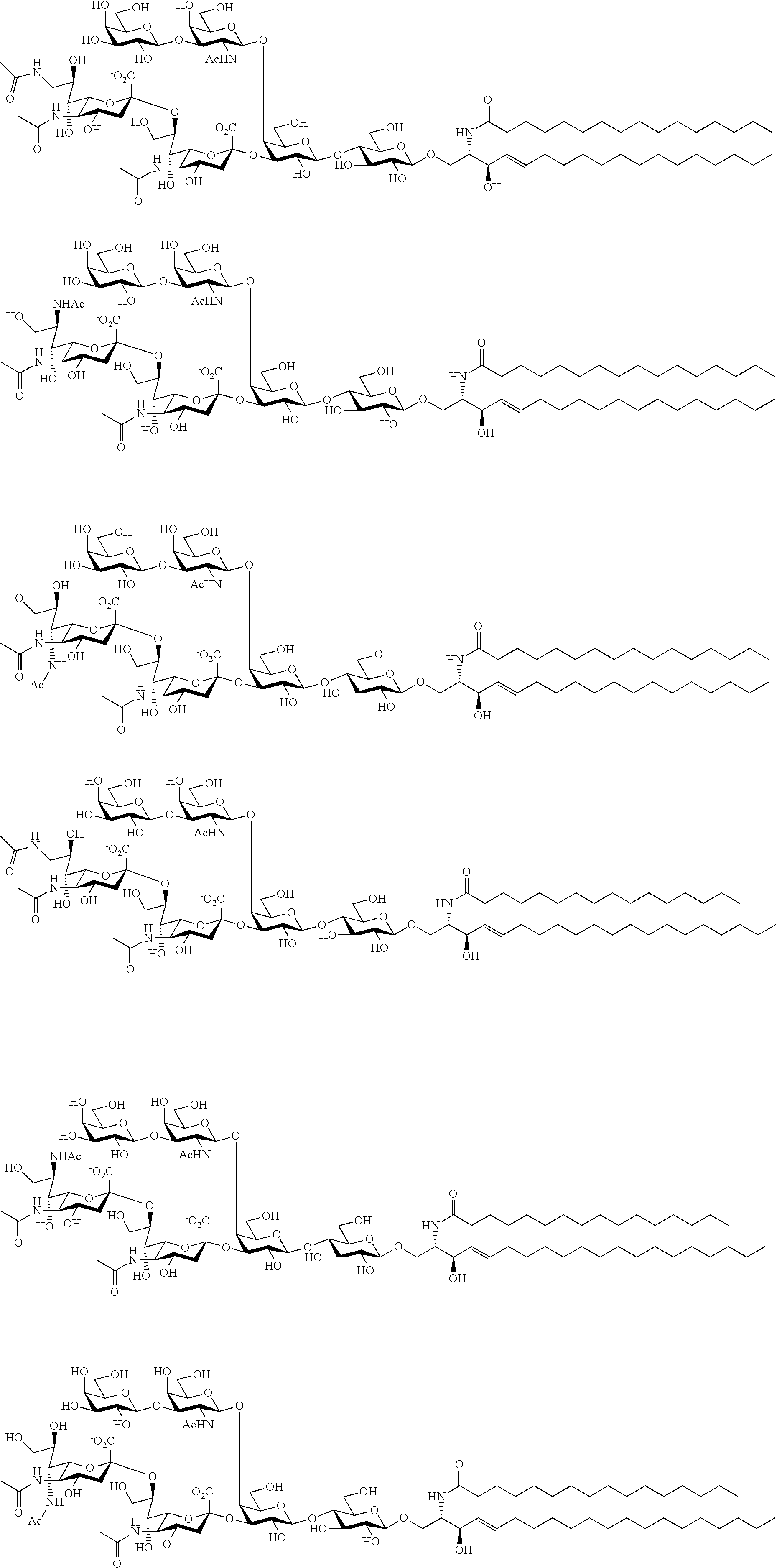

15. The compound of claim 1, wherein the compound is selected from the group consisting of:

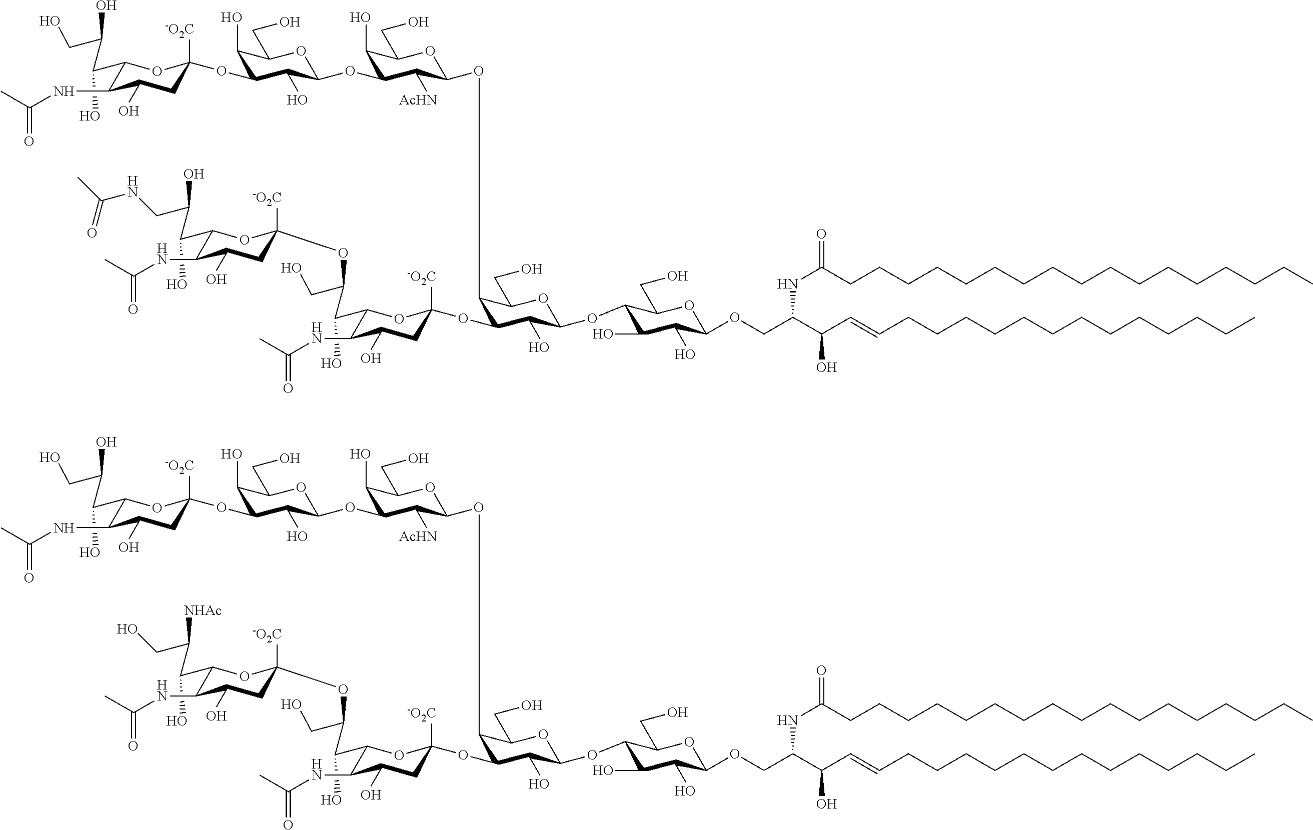

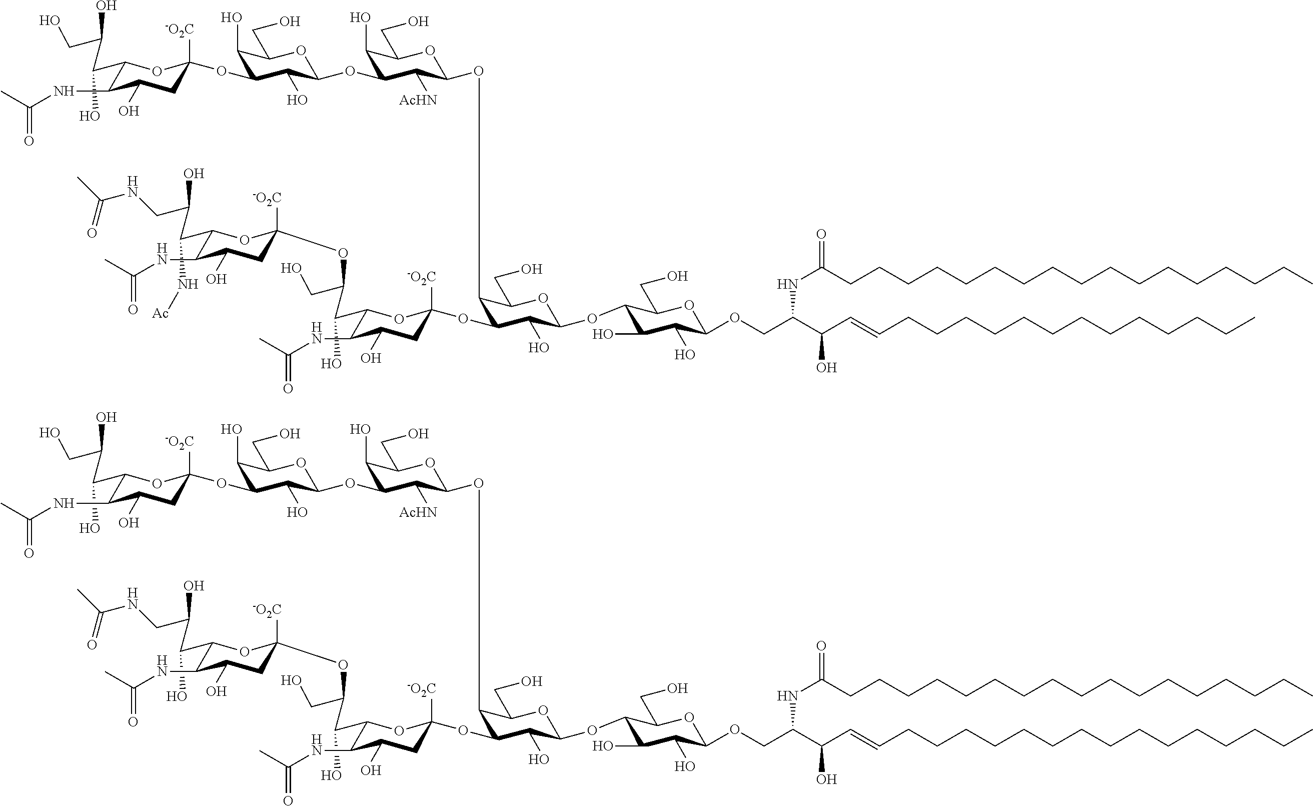

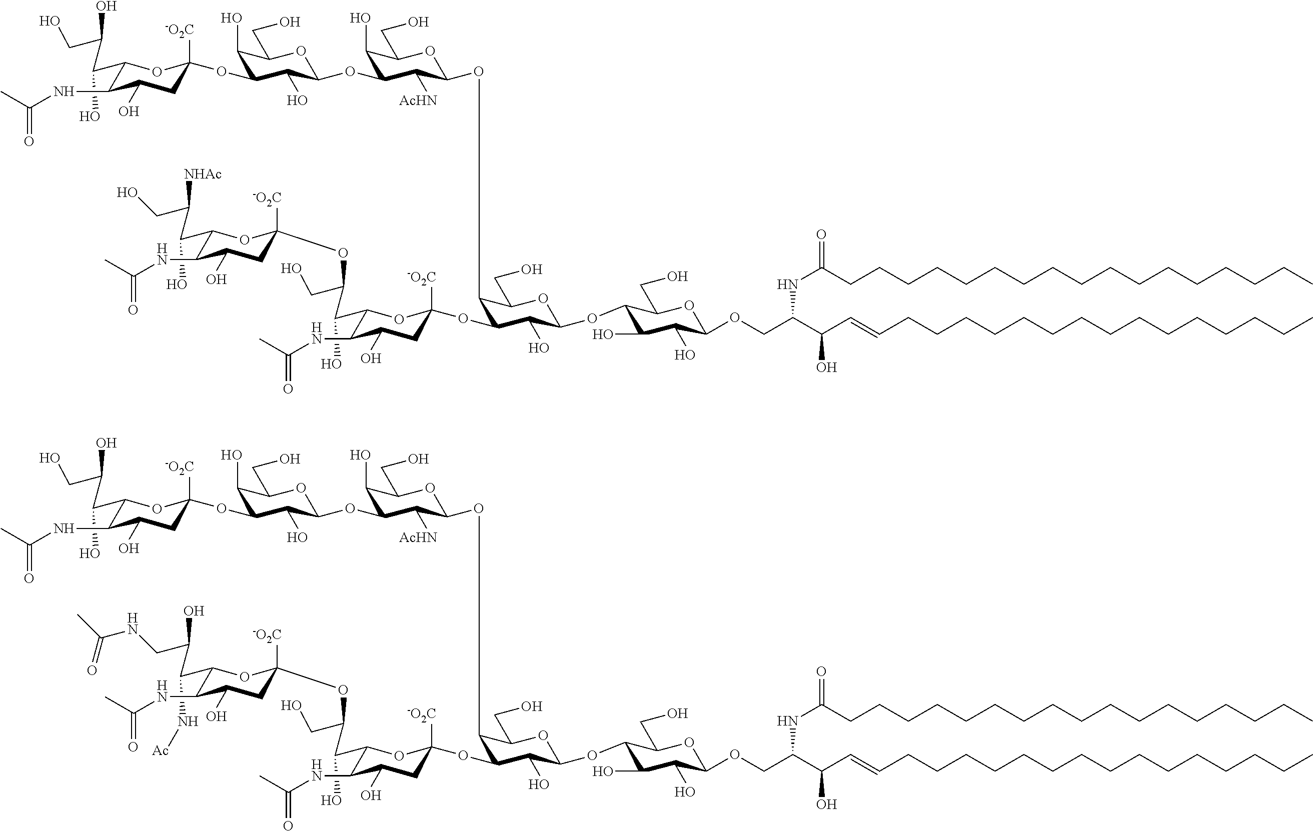

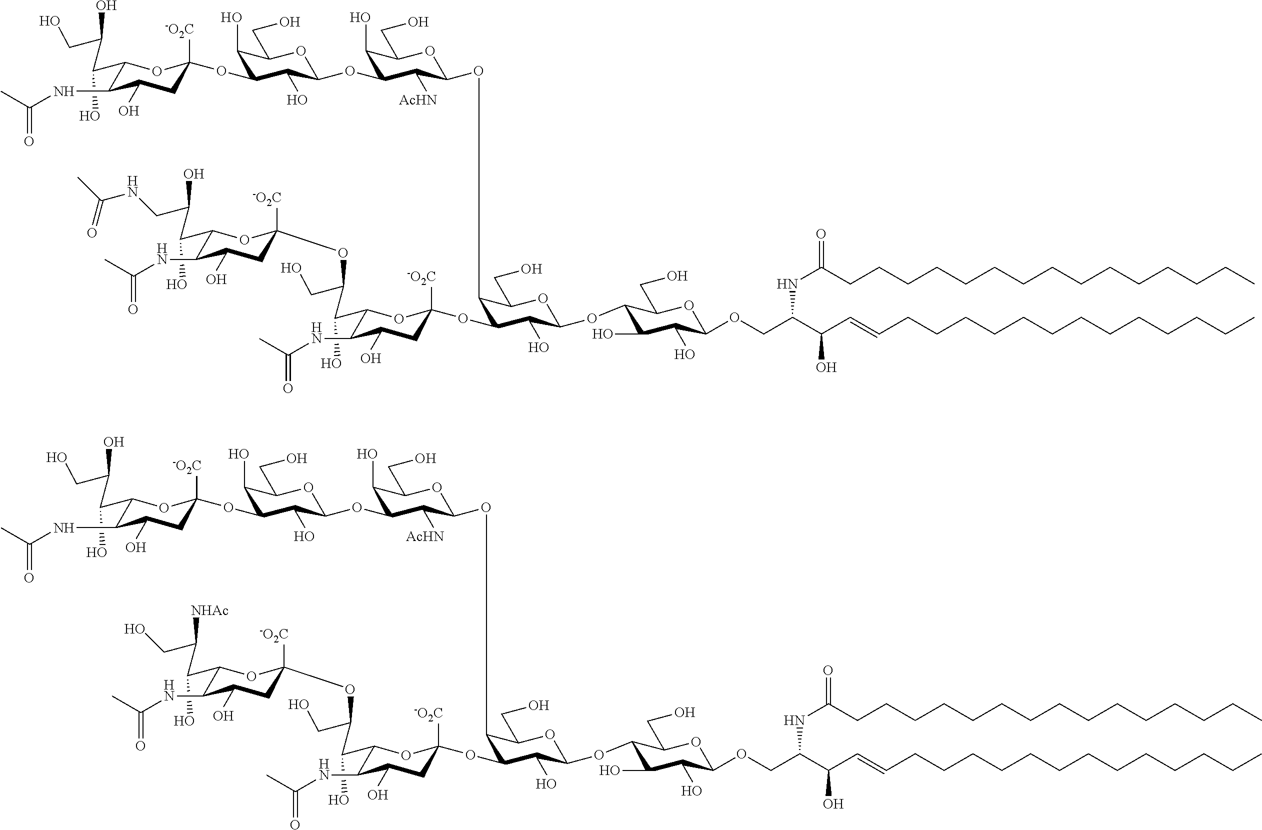

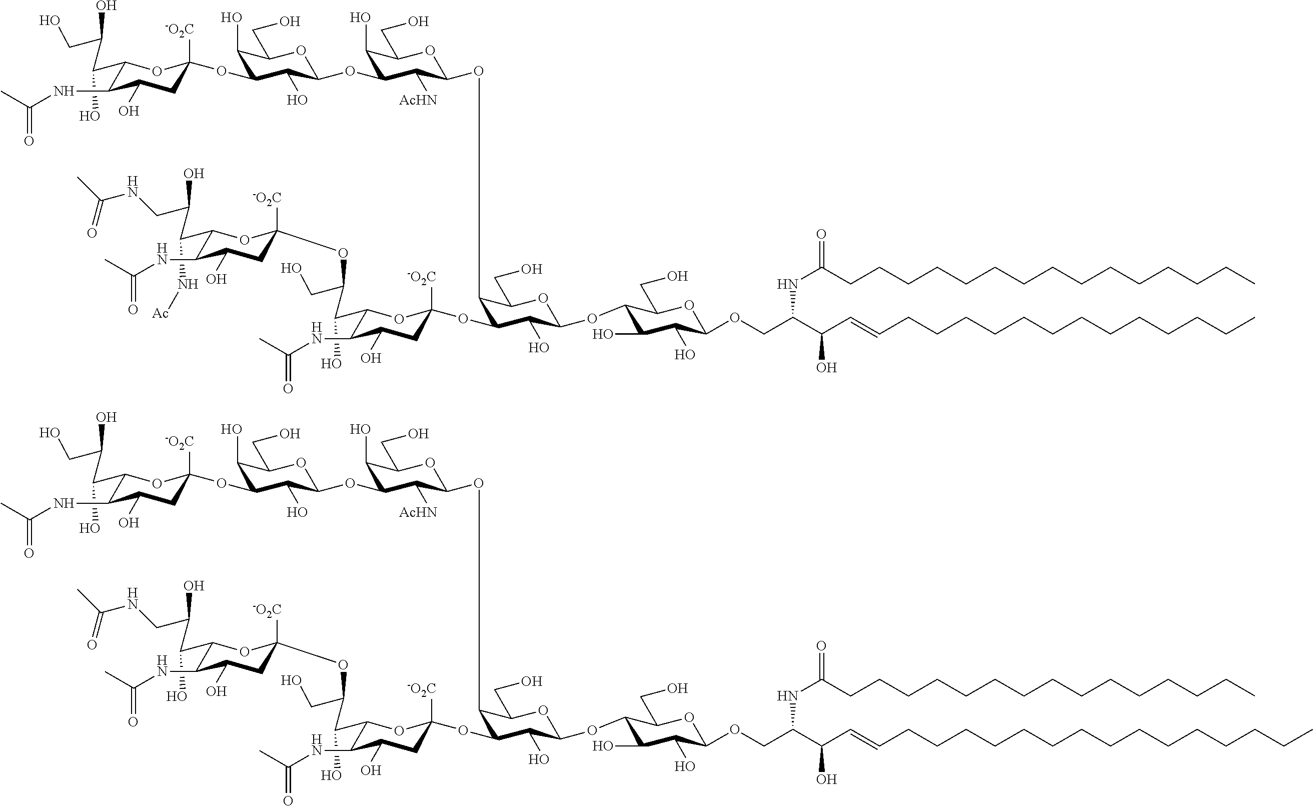

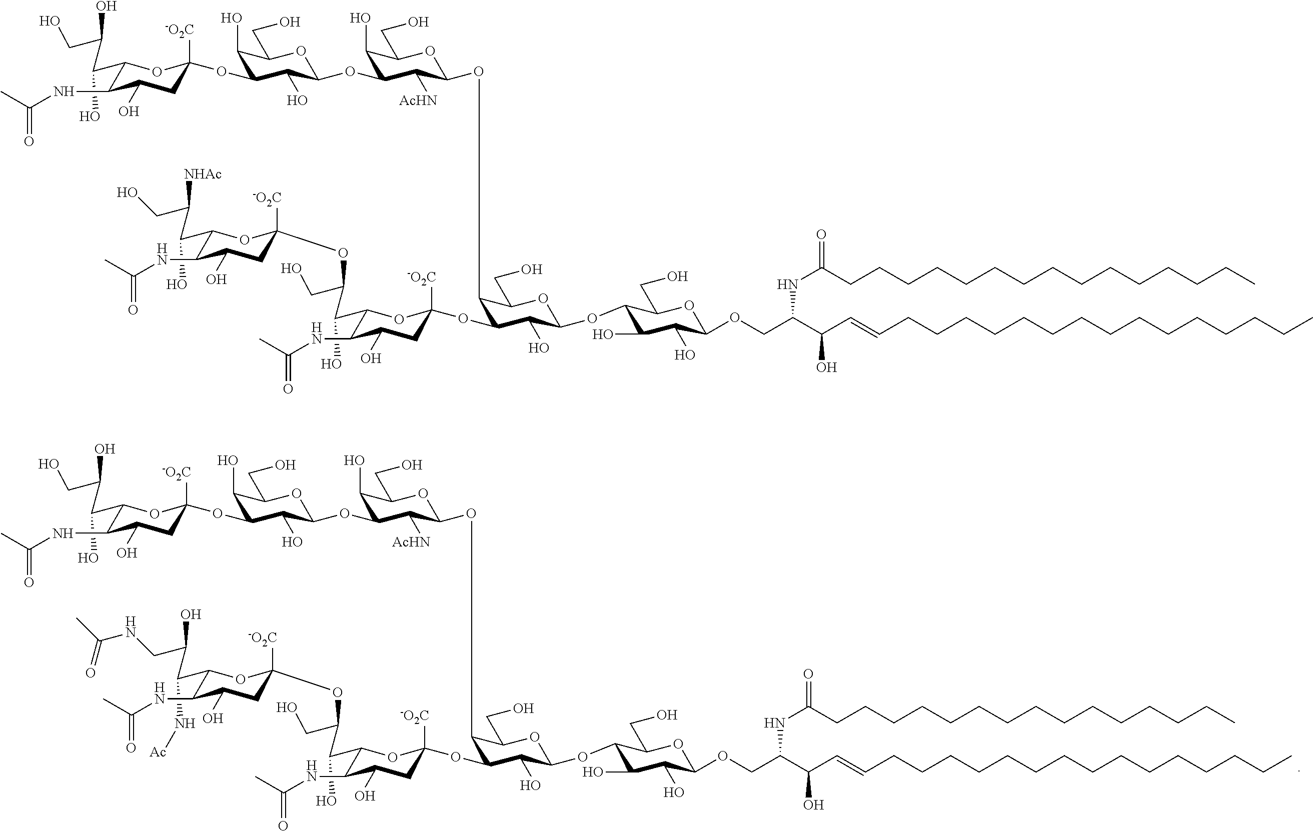

16. The compound of claim 1, wherein the compound is selected from the group consisting of:

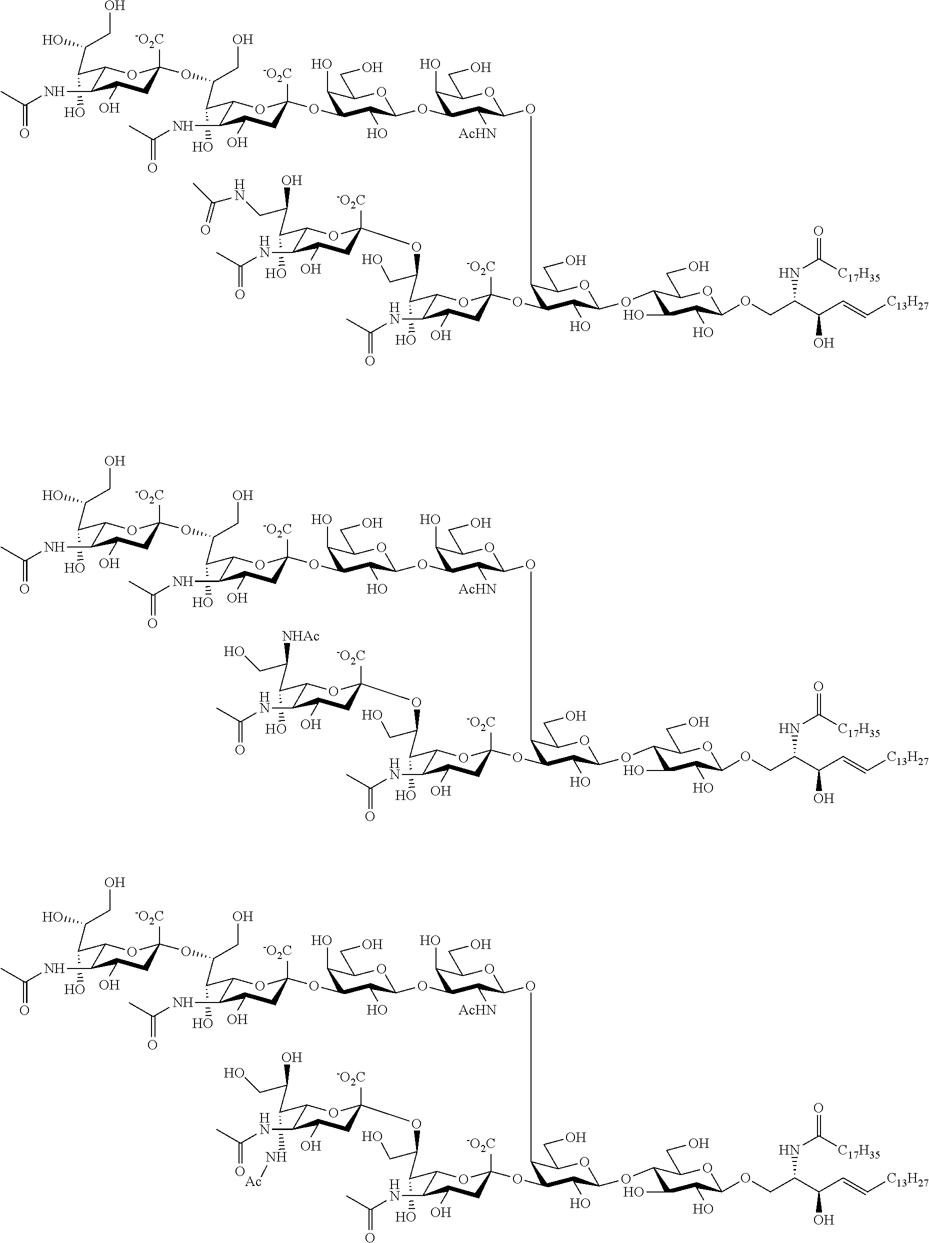

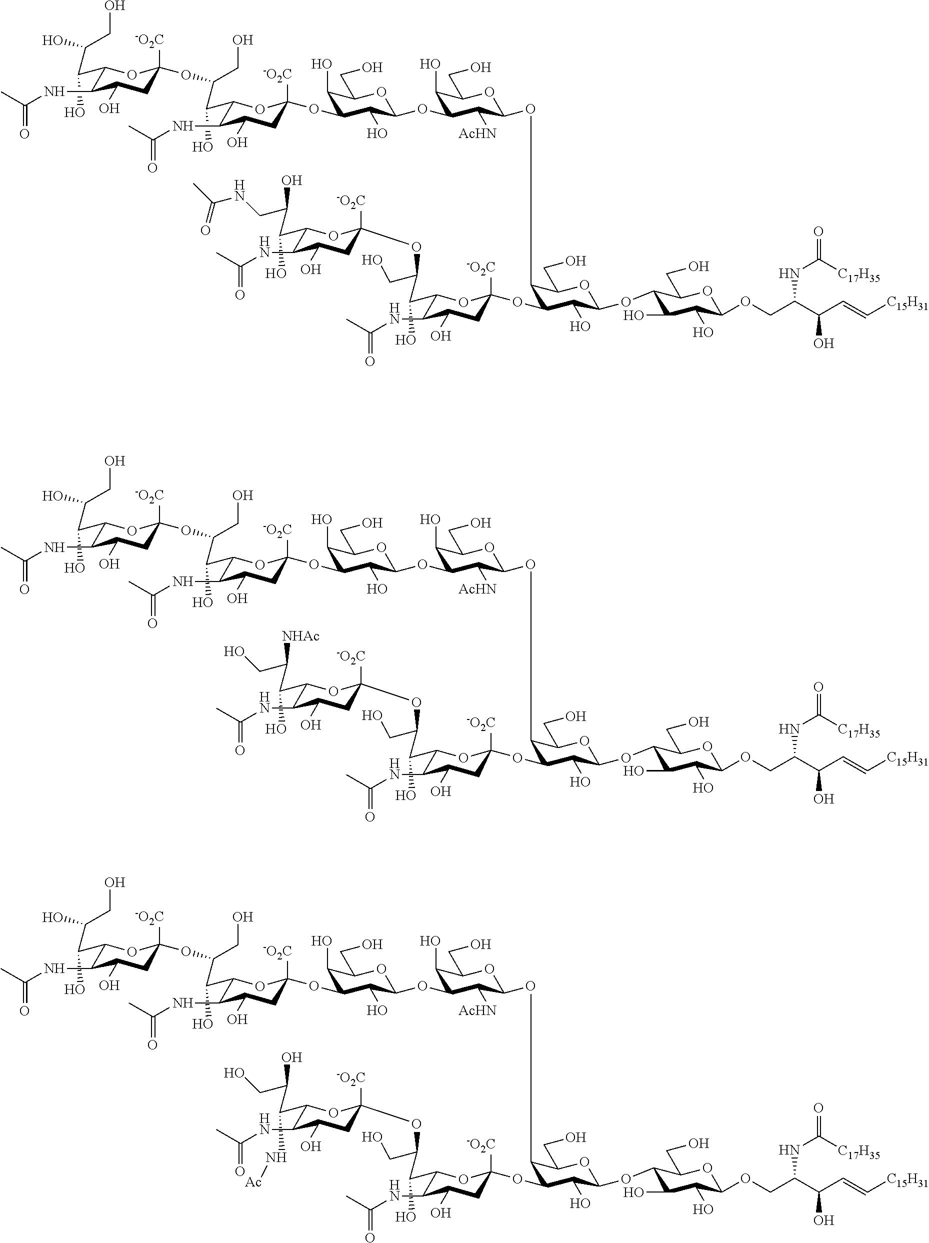

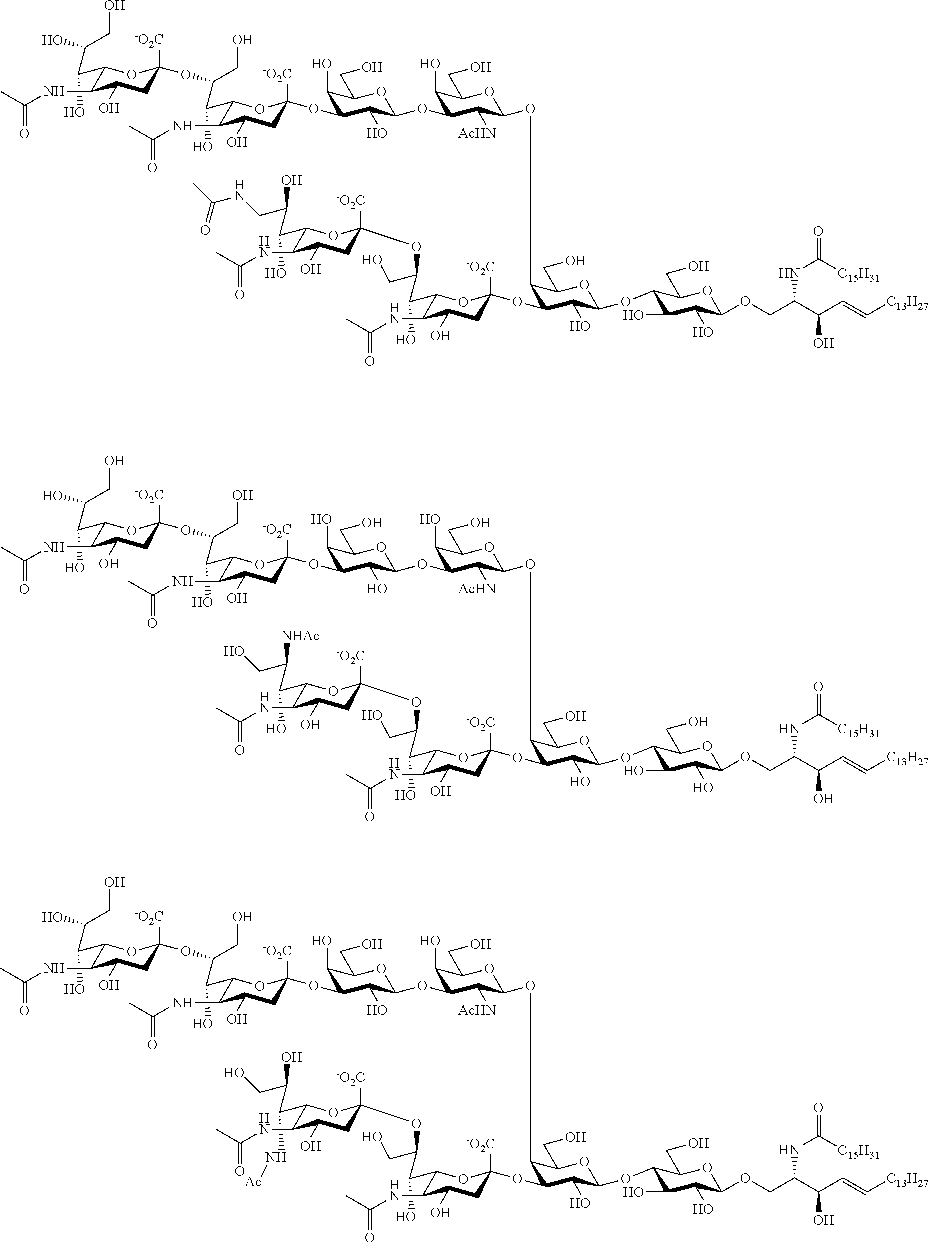

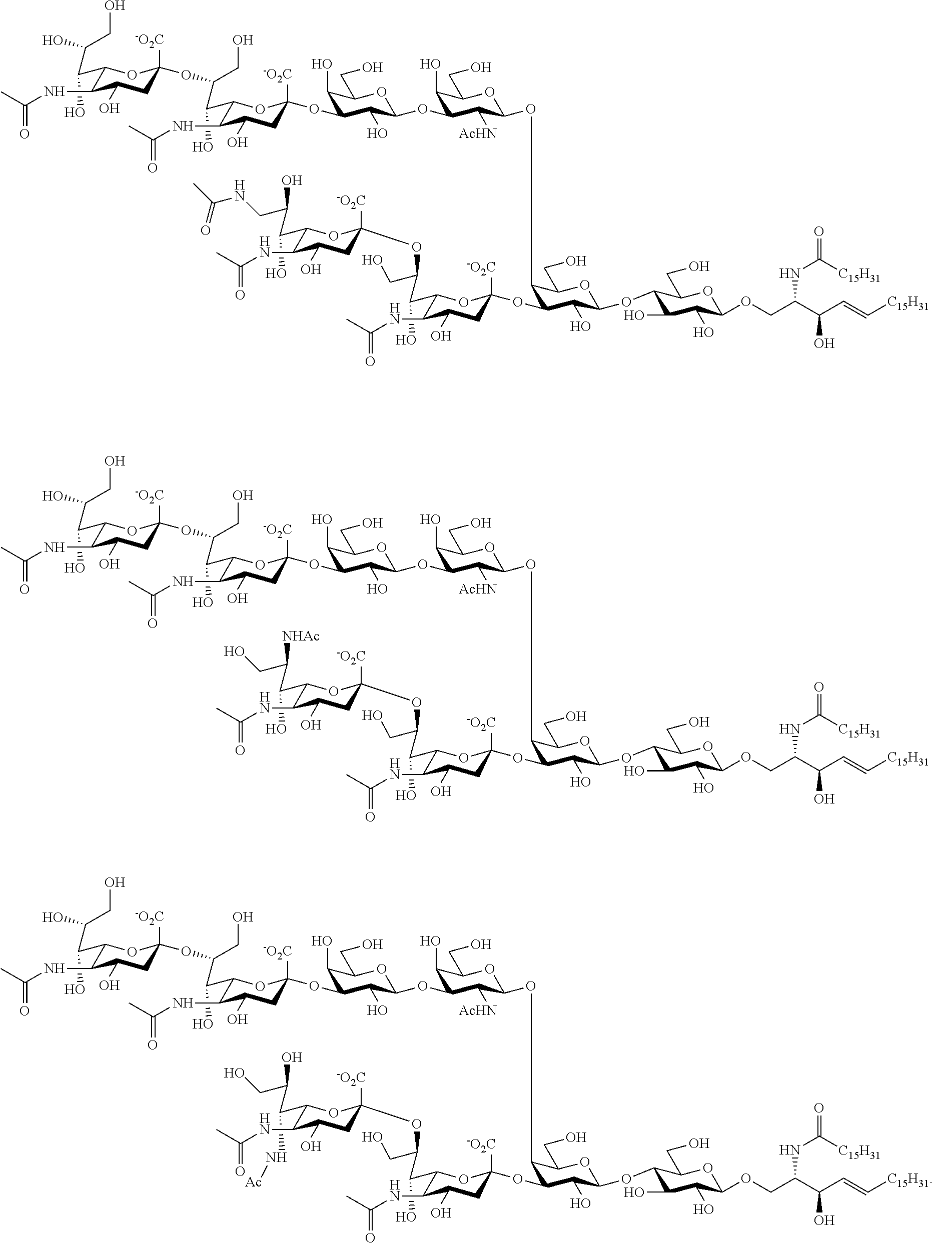

17. The compound of claim 1, wherein the compound is selected from the group consisting of:

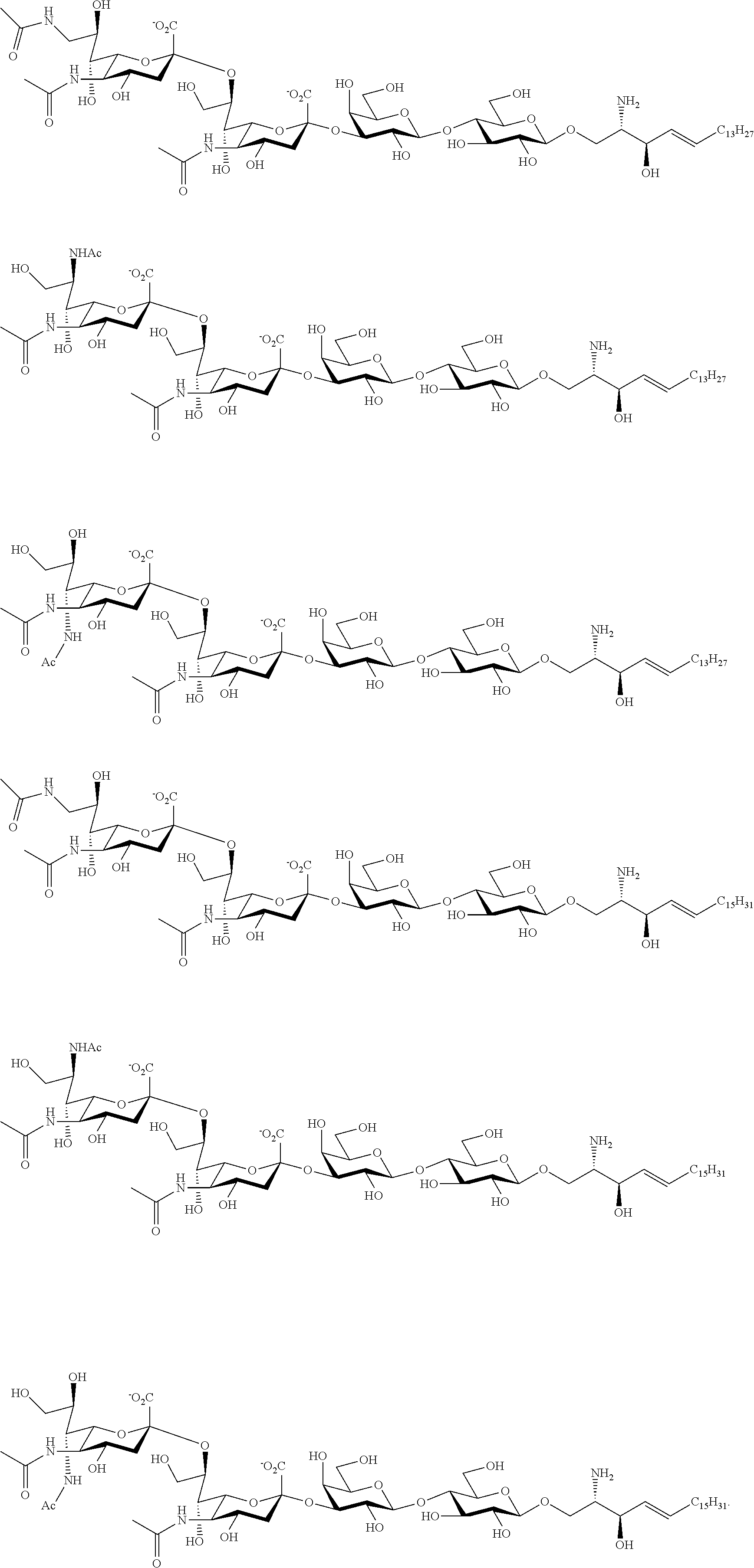

18. The compound of claim 1, wherein the compound is selected from the group consisting of:

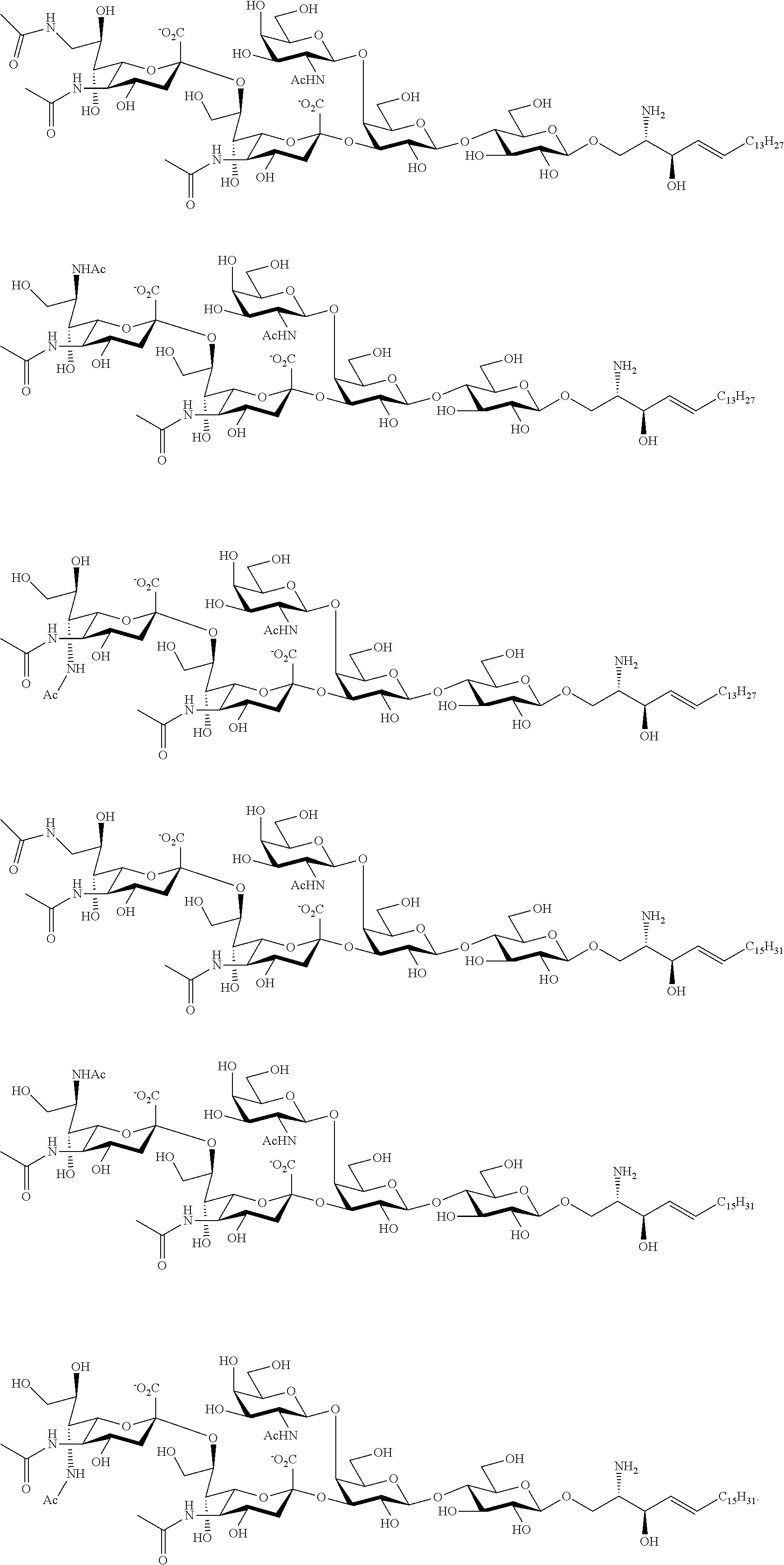

19. The compound of claim 1, wherein the compound is selected from the group consisting of:

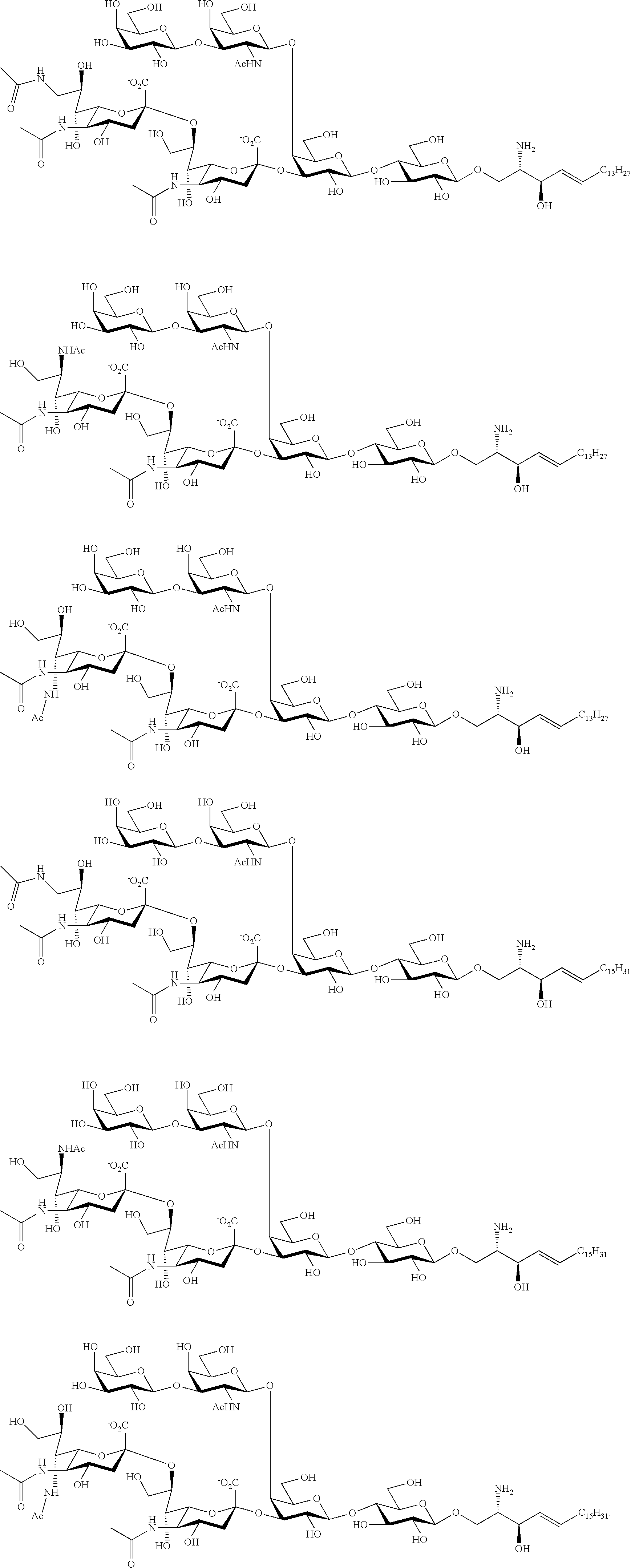

20. The compound of claim 1, wherein the compound is selected from the group consisting of:

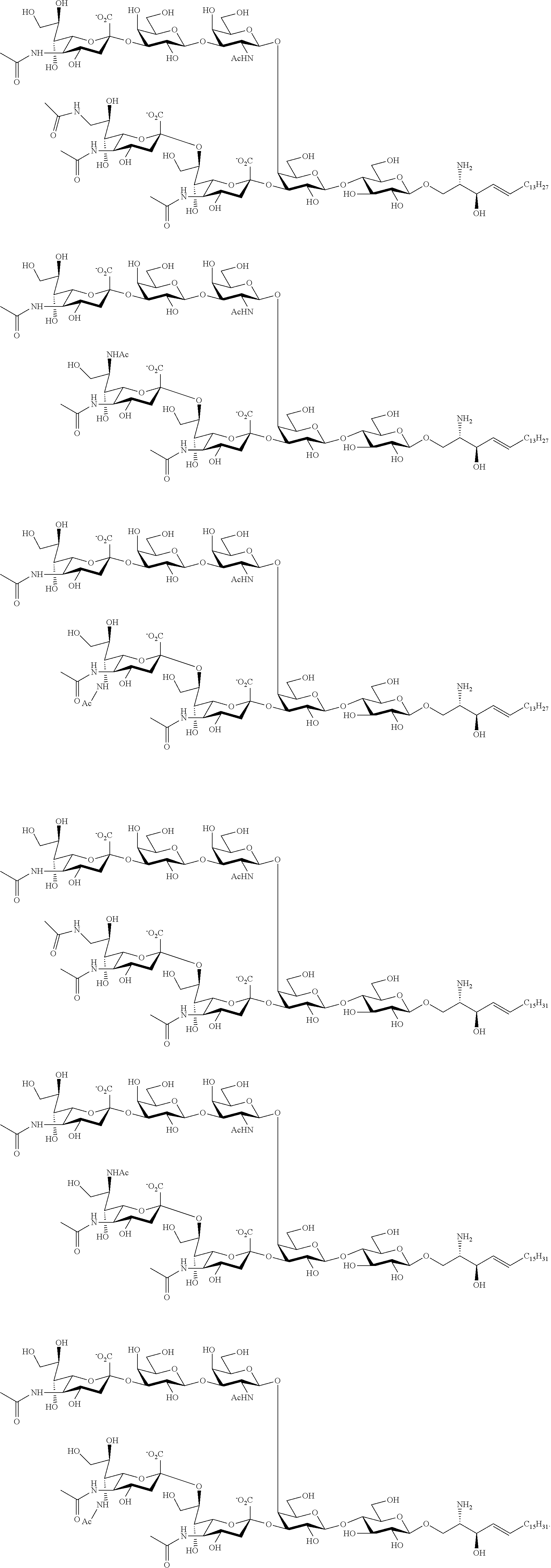

21. The compound of claim 1, wherein the compound is selected from the group consisting of:

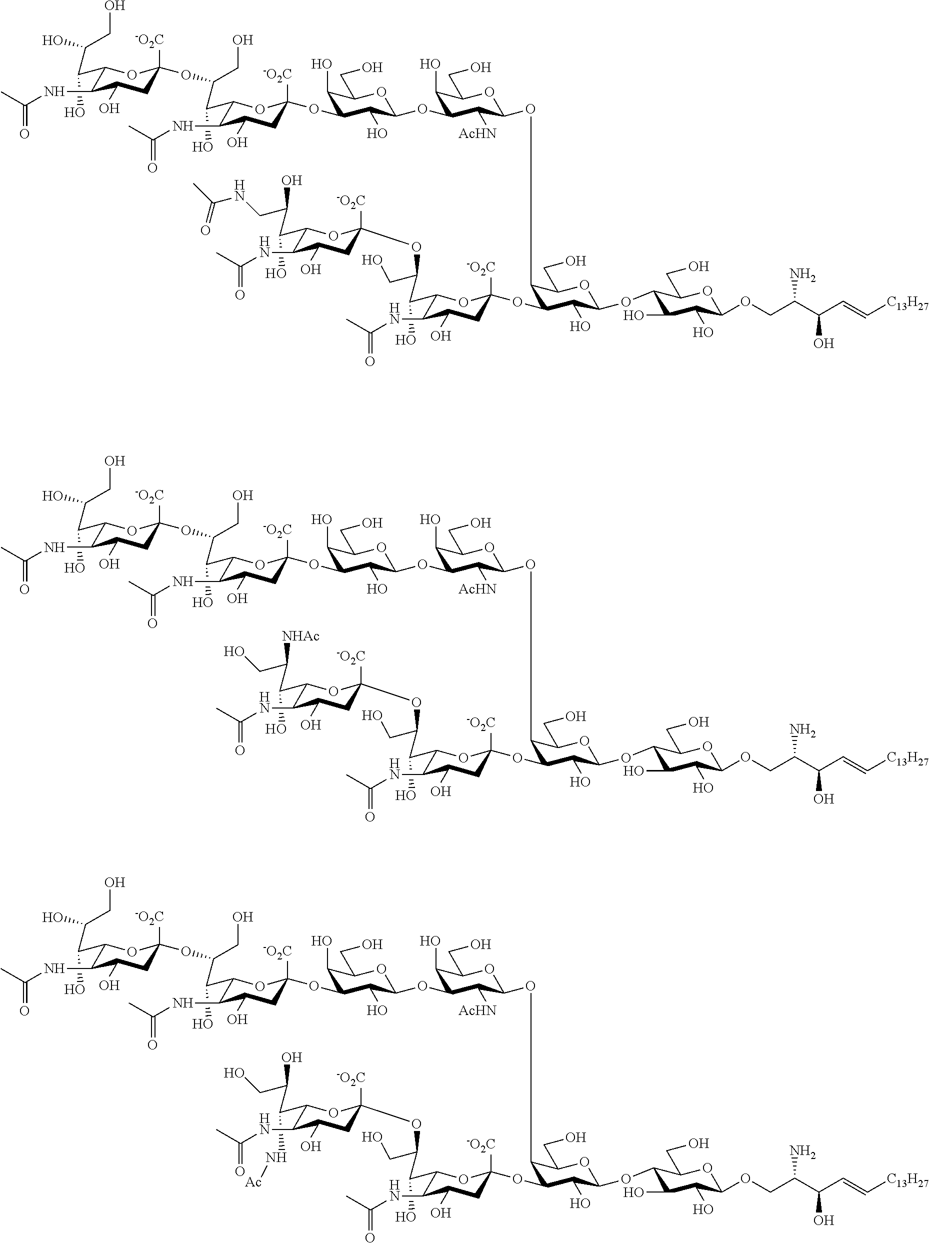

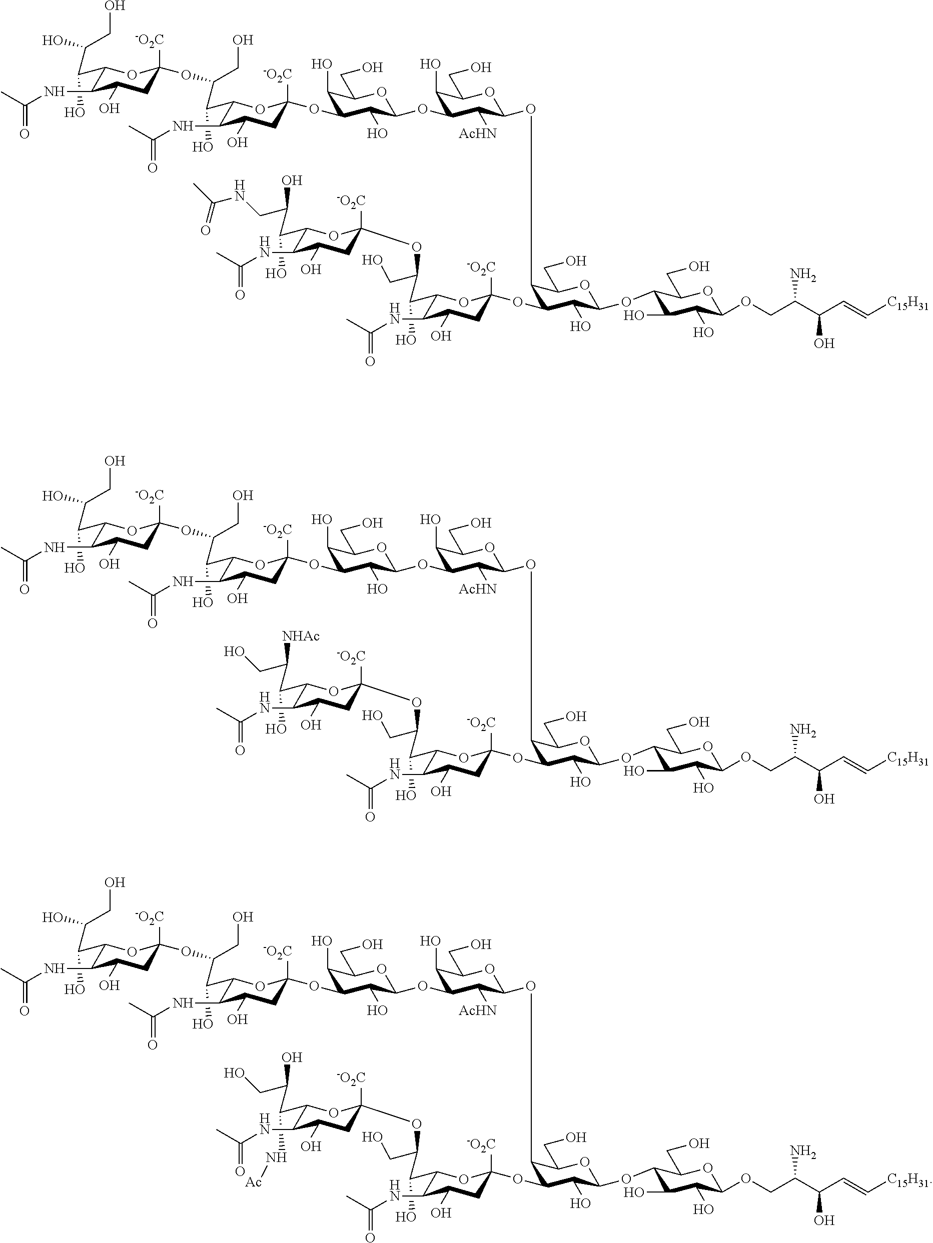

22. A method of synthesizing an N-acetyl-containing b-series ganglioside, comprising:
step (i) forming a reaction mixture comprising a donor precursor, cytidine 5′-triphosphate, pyruvate, a sialic acid aldolase, a CMP-sialic acid synthetase, a sialyltransferase, and an acceptor comprising an a-series sphingosine, under conditions sufficient to result in the sialylation of the a-series sphingosine to form a b-series sphingosine of the following formula: 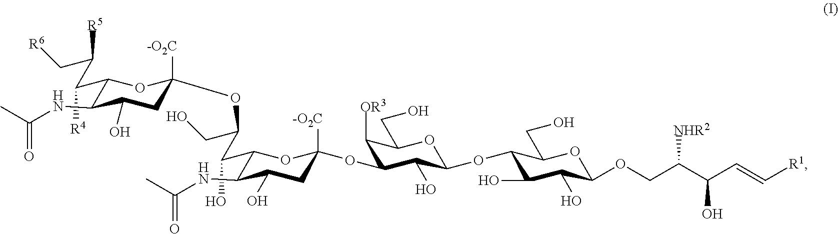
or a salt thereof, wherein R¹ is an alkyl chain; R² is H; R³ is H, a monosaccharide, or an oligosaccharide; and R⁴, R⁵, and R⁶ are each independently selected from OH, NHAc, or N₃, wherein at least one of R⁴, R⁵, and R⁶ is NHAc or N₃; and
step (ii) acetylating the b-series sphingosine to form a b-series ganglioside of Formula I: 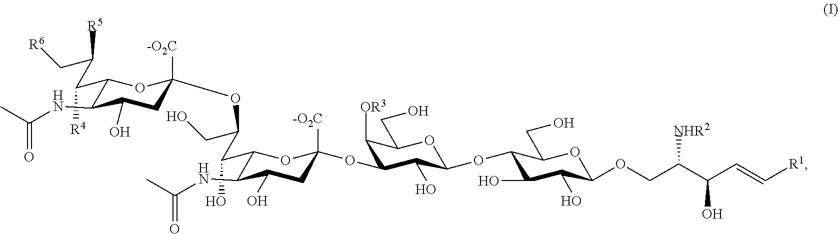
or a salt thereof, wherein R¹ is an alkyl chain; R² is —C(O)alkyl; R³ is H, a monosaccharide, or an oligosaccharide; R⁴, R⁵, and R⁶ are each independently selected from OH, NHAc, or N₃, wherein at least one of R⁴, R⁵, and R⁶ is NHAc or N₃.

23. The method of claim 22, wherein the donor precursor comprises an N-acetyl group or an azido group.

24. The method of claim 22, wherein the donor precursor is 6-acetamido-6-deoxy-N-acetylmannosamine (ManNAc6NAc), 6-azido-6-deoxy-N-acetylmannosamine (ManNAc6N₃), 4-acetamido-4-deoxy-N-acetylmannosamine (ManNAc4NAc), 4-azido-4-deoxy-N-acetylmannosamine (ManNAc4N₃), 5-acetamido-5-deoxy-N-acetylmannosamine (ManNAc5NAc), or 5-azido-5-deoxy-N-acetylmannosamine (ManNAc5N₃).

25. The method of claim 22, wherein the acceptor comprises GM3βSph.

26. The method of claim 22, wherein the sialic acid aldolase is P. multocida sialic acid aldolase (PmAldolase) or Escherichia coli sialic acid aldolase (EcAldolase).

27. The method of claim 22, wherein the CMP sialic acid synthetase is Neisseria meningitidis CMP-sialic acid synthetase (NmCSS) or Legionellapneumophila CMP-5,7-di-N-acetyllegionaminic acid synthetase (LpCLS).

28. The method of claim 22, wherein the sialyltransferase is Campylobacter jejuni α2-3/8-sialyltransferase (CjCst-II).

29. The method of claim 22, further comprising one or more glycosylation steps following step (i).

30. The method of claim 22, wherein when the donor precursor is 6-azido-6-deoxy-N-acetylmannosamine (ManNAc6N₃), 4-azido-4-deoxy-N-acetylmannosamine (ManNAc4N₃), or 5-azido-5-deoxy-N-acetylmannosamine (ManNAc5N₃), the method further comprises a step of reducing the azido group to an amino group after step (ii).

31. The method of claim 22, wherein when the donor precursor is 6-azido-6-deoxy-N-acetylmannosamine (ManNAc6N₃), 4-azido-4-deoxy-N-acetylmannosamine (ManNAc4N₃), or 5-azido-5-deoxy-N-acetylmannosamine (ManNAc5N₃), the method further comprises a step of converting the azido group to an acetamido group after step (ii).
